SEQUENCE LISTING (189 sequences)

SEQ ID NO: 1 - protein, 98 aa, Homo sapiens
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVS

SEQ ID NO: 2 - protein, 45 aa, Homo sapiens
RTLVTFKDVFVDFTREEWKLLDTAQQIVYRNVMLENYKNLVSLGY

SEQ ID NO: 3 - protein, 36 aa, Homo sapiens
MAAAVRMNIQMLLEAADYLERREREAEHGYASMLPY

SEQ ID NO: 4 - protein, 58 aa, Homo sapiens
GASQCMPLKLRFKRRWSEDCRLEGGGGPAGGFEDEGEDKKVRGEGPGEAGGPLTPRRV

SEQ ID NO: 5 - protein, 13 aa, herpes simplex virus 7
DALDDFDLDMLGS

SEQ ID NO: 6 - protein, 55 aa, Artificial Sequence
  Synthetic construct
GRADALDDFDLDMLGSDALDDFDLDMLGSDALDDFDLDMLGSDALDDFDLDMLIN

SEQ ID NO: 7 - protein, 102 aa, Homo sapiens
KGFGAFERSILTQIDHILMDKERLLRRTQTKRSVYRVLGKPEPAAQPVPESLPGEPEILPQAPANAHLKDLDEEIFDDDDFYHQLLRELIERKTSSLDPNDQ

SEQ ID NO: 8 - protein, 31 aa, Homo sapiens
PGLPNGLLSGDEDFSSIADMDFSALLSQISS

SEQ ID NO: 9 - protein, 48 aa, Homo sapiens
PYTPNLPHHQNGHLQHHPPMPPHPGHYWPVHNELAFQPPISNHPAPEY

SEQ ID NO: 10 - protein, 100 aa, Homo sapiens
PPHLNPQDPLKDLVSLACDPASQQPGPLNGSGQLKMPSHCLSAQMLAPPPPGLPRLALPPATKPATTSEGGATSPTSPSYSPPDTSPANRSFVGLGPRDP

SEQ ID NO: 11 - protein, 68 aa, Homo sapiens
ADFQPPYFPPPYQPIYPQSQDPYSHVNDPYSLNPLHAQPQPQHPGWPGQRQSQESGLLHTHRGLPHQL

SEQ ID NO: 12 - protein, 112 aa, Homo sapiens
NRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQT

SEQ ID NO: 13 - protein, 143 aa, Homo sapiens
QGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQ

SEQ ID NO: 14 - protein, 95 aa, Homo sapiens
DLQQLQQLQQQNLNLQQFVLVHPTTNLQPAQFIISQTPQGQQGLLQAQNLLTQLPQQSQANLLQSQPSITLTSQPATPTRTIAATPIQTLPQSQS

SEQ ID NO: 15 - protein, 63 aa, Homo sapiens
QLAGDIQQLLQLQQLVLVPGHHLQPPAQFLLPQAQQSQPGLLPTPNLFQLPQQTQGALLTSQP

SEQ ID NO: 16 - protein, 90 aa, Artificial Sequence
  synthetic construct
NLFQLPQQTQGALLTSQPNLFQLPQQTQGALLTSQPNLFQLPQQTQGALLTSQPNLFQLPQQTQGALLTSQPNLFQLPQQTQGALLTSQP

SEQ ID NO: 17 - protein, 91 aa, Homo sapiens
PPSTGNSASLSLPLVLQPGLSEPPQPLLPASAPSAPPPAPSLGPGSQQAAFGNPPALLQPPEVPVPHSTQFAANHQEFLPHPQAPQPIVPG

SEQ ID NO: 18 - protein, 111 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKK

SEQ ID NO: 19 - protein, 88 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPA

SEQ ID NO: 20 - protein, 11 aa, Human immunodeficiency virus
YGRKKRRQRRR

SEQ ID NO: 21 - protein, 12 aa, Artificial Sequence
  Synthetic construct
PVRRPRRRRRRK

SEQ ID NO: 22 - protein, 12 aa, Artificial Sequence
  Synthetic construct
THRLPRRRRRRK

SEQ ID NO: 23 - protein, 9 aa, Artificial Sequence
  Synthetic construct
RRRRRRRRR

SEQ ID NO: 24 - protein, 16 aa, Drosophila melanogaster
RQILIWFQNRRMKWKK

SEQ ID NO: 25 - DNA, 2500 bp, Homo sapiens
ggcttccagaggacaaaccccaatacaggagaagcacaagacagagaagggccaatggggtcatcccctccctaacgagactctctgtgctgggggtgctaattacatggcaggaagaatggggcctctaaggggagtgtggggtctgtctctcccttttttccatctttttcctctctcgctttctttcttacacagaaacatacacataccgagaaacctatttctcagacccctttttctcctctgtctttctctctccctctcccacacctcacacacacatactcccacttgcaactattctgtttctctcctgggctcccccactttcccttccccaccccacttgtatgctctggaatctgtggagacgccagccctgcccaatcagagatgccaaaaatggggacatgacttctggacagaggacatgggccacgcccccatgcatccccacccccgcccctccggacggcttacttacctcatacgcagctcatcttaaaccaatagaatcgctcggtggacgagagtgtctgactcagatatctacctcggagggagtttctgctactttagggaattattgactgggctttggggttgaacttttttttttttaaagaaagaaaaagaaaccctgggatccatctgttttttttgttgttgttgttgtttttgttgttgttggtggtggtggtggtggtggttcttaatttttaatttagtttggggaagtagcttgtttttttttttataaatatgttgatttcttgtctttttttttttttatttcttactttcccatattaggggtgatagccaaaggggttctggtaagagaaagggggacaaacagaactggtaaagaggcccccctggctccaggcctgtccatcaggaagtaaattttacagggcaccaagctttgccccctaaaatcccttaggtgttctttgttcatgcaggcaggtttctgccgcatttgatgtggaggcagtgaagggcttgccctgctggcctctcatcccccttcttcccacaacccttgggcagggctggactcagtaattttgaggaaattgaagatgccatcttcccctgtgagtgacatgtctttaattttttaaaaaactactatttgaaaattggagggggaagaatgggaagggagttattgccaaatatgttaaatatgggttggggtgcttgtatatgtatcttcctcaatttccccataaatgaggtatctttttgtcacaccaaaatcaaggggtagggagagggaggaggttgcaaaaagccagatgtgggggaaaagtaacatcaacactgtcccatcctcagccctgaactagctaccatctgatcccctcagacattctcaggattttacaagactgtcagagtggggaacccctcccattaaagatccgggcaggactggggacaggttggaagtgtgatgggtgggggggtgggaggcatgggccgggggcagttctctcctcacttgtaaacttgtgtagtttcacagaaaaaaaacaaaatgcagttttaaataaagaaatttcttttttccctgggtttagttgagaatttttttcaaaaaacatgagaaaccccagaaaaaaaatgattttctttcacgaagttccaaacaggtttctctcctgttccccagccttgccttcatgatgcaggcccaattgcacccttgcagacaacagtctggcctgaaccctattgatgcaactttgcgcaatcaagatggggctccagtgggtcaccaggcagccctgatggactgatggaataaataggatcgggggctctgagggaatgagaccctagagggtacactccccatcccccagggaagtgactgtacccagaggctggtagtacccaggggtggggtgataattatttctctagtacctgaaggactcttgtcccaaaggcatgaattcctagcattccctgtgacaagacgactgaaagatgggggctggagagagggtgcaggccccacctagggcggaggccacagcagggagaggggcagacagagccaggaccctggaaggaagcaggatggcagccggaacagcagttggagcctgggtgctggtcctcagtctgtggggtgagccactccctcaaccccactgaccctccctgcagaaagcactttaaccccacaccccagtcgtcctagaacttttcccagaacccgaggaagtgcctttcaaggtccctcacccaccctgtccaaattttgttagccctcattcccttcctacccctctaccatggtgctatctcccaggggcagtagtaggtgctcaaaacatcacagcccggattggcgagccactggtgctgaagtgtaagggggcccccaagaaaccaccccagcggctggaatggaaactggtaagcggggctcctgttgcagcctcccaacttccagggagaccagcaatgatttgga

SEQ ID NO: 26 - DNA, 2500 bp, Homo sapiens
agttatcagcacactccaggtactagccaaggactacaaaatcaacgtgaatgtcagcttttgtatcaaaagctcaaaggagaaactcaaactttacatagatgtcccatgaagatgttcagcaaacccattcttctctgttccctggaatccatcccagtattgtgctatgtgtgtgtctagtaattctttacaaaaagctctgtttcttgtgatgctatcagatcacattgaagaatatacaagccgtactatgaaggctgttgtctcatatagtcctaacgtagtgagaactgatgttcttacatgctgtctttttgggcactcaaagaaattcctgtacagtcttacaaatcagttgtagcttaaattgatttgtgttgtgacttgtacacacaggtcacattcccttgacagaaaatatagtttaaaaccaaatttgcagcccttgttaagtgaatgcacaggactttattgtattcaggtcttttattgtaagactcactcctgtcttcattttatgttccactgttgtgcttcccatttgcctttctctagttttgttttctgtgtttctacggactgctctcagcccaggtgtgcaggaagcacacacatgcctgcagagccttcatggcctctgcattcagggcatgacttcaacgcacagtggctgtactgatttgttaaaacaaaggaacagattacttctcctaattcacagggaagttccaggttgtgcgggcagtgagcagacctgtgtctgtctgcgcttgccctggtgaaaaaccccaccgttcaggctgcagggtgcgagacccaggcacaaacattttgctggatgaggaggaaagatgtaaggttgctccccttcagagacagcaaagggcaggtctgtagcttcacttacttcaggattgtgatttttgacagagccgagagatcagggttgttgaaccaggcctgaaggtcctagtgaatctcgtgaagagaggaggggtctggctgtaacatggacctagaggacatttttactgcaggagaaggaacagtggggatggggtggacttgccaaaggaatatagctcaagttcctgcagcccaaaaaagctcagtttcttttggccaaagcttccgcgagtttccctggcatttctcctgcgggagctacaggggcagtgggacacttagcctctctaaaagcacctccacggctgtttgtgtcaagcctttattccaagagcttcacttttgcgaagtaatgtgcttcacacattggcttcaaagtacccatggctggttgcaataaacattaaggaggcctgtctctgcacccggagttgggtgccctcatttcagatgatttcgagggtgcttgacaagatctgaaggaccctcggactttagagcaccacctcggacgcctggcacccctgccgcgcgggcacggcgacctcctcagctgccaggccagcctctgatccccgagagggtcccgtagtgctgcaggggaggtggggacccgaataaaggagcagtttccccgtcggtgccattatccgacgctggctctaaggctcggccagtctgtctaaagctggtacaagtttgctttgtaaaacaaaagaagggaaagggggaaggggaccctggcacagatttggctcgacctggacataggctgggcctgcaagtccgcggggaccgggtccagaggggcagtgctgggaacgcccctctcggaaattaactcctcagggcacccgctcccctcccatgcgccgccccactcccgccggagactaggtcccgcgggggccaccgctgtccaccgcctccggcggccgctggccttgggtccccgctgctggttctcctccctcctcctcgcattctcctcctcctctgctcctcccgatccctcctccgccgcctggtccctcctcctcccgccctgcctccccgcgcctcggcccgcgcgagctagacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaaggtaagggcgtgtctcgccggctcccgcgccgcccccggatcgcgccccggaccccgcagcccgcccaaccgcgcaccggcgcaccggctcggcgcccgcgcccccgcccgtcctttcctgtttccttgagatcagctgcgccgccgaccgggaccgcgggaggaac

SEQ ID NO: 27 - DNA, 2500 bp, Homo sapiens
tgatgtgtagtaattaaaatgcaggaggcctaatgggtgggcagcttacataggagtataaaccaagcttgaccaggagctgaaaggttaaatggtggctcttaggggaaaaccctataaacagtggctgaagttcatttattcaacaaagatatgagttcttgtttctcattttttgttttgtattattttgttttgagacagggtcttactctgtcgcccaggctggagtgtagtggctggatcatagctcactgcagcctcaaactcctgggctcaagccatcctccttcttcagcctccacctccagctaatttttaaaaatattttgtagagacaagggctcactttgtttcccaggctggtcttgaacttctggcttcaagtgatcctcccgcttcggccacccaaagtgctgggattacaggcgtgagctgtaatttagttgtttatttactcatttgttcaacaaatacttattgaatatttgctctttggccagtcaagggatttcatgagtgtctactatgtgaataacactgtgttggccactagtctgtcacctactggtggattagaaaaatagcgcgaggaccattttttcttttcttttctttttttttttgagacggagtcttgctctgttgccaggctggagtgcagtggcacaatctcggctcactgcaacctccgcctcccgggttcaagcgattcctctgccgcagcctccccagtagctgggattacaggcaagcgccaccatgcctggctaatttttttgtattttagtagagacggggtttcaccttgttggcaaggatagtctcgatctcccgacctcgtgatccacccgcctcggcctcccaaagtgctgggattacaggcataagccaccgcacccggccaactcttttcttaaattagccagggaggcgtgggtgggttgggtgaggagttgggtggggggatctcattcagtattcaaacttctacaagtttcggggttgaggtgggtgatggtaagggaacaggccctgccactacctttcatagtgacttccatttgtgtaatatttttggtccactgagagctattattttatttgattcttatgaccatcttgtgaaggagtatcaacagataccccgttttgattttatcagatgcatgatttgtcctacatcaaacttcataaatgatggacagaatggaggaatccttcagaccaagtgctgcctacttcccaccccaatggtggcctcagcctgggctcacatcacacgccccaaggagccttggaaaaaataaaggctcttggctccttcctgggacagcgtgattcctcatgtctgagcaggcccatgaacttgtatttttcagacgttccctaggacccgtgtccatctggattagggaaccactacattataccacttcgcgggaagactcagggggaagcattttagccactttcctgtgttccacagtactggagggtgttctgagtgggctgtgattaatttccaaaccaaccacacgtctcccctcaactcccactgcttactctttgcttcctagacattcactgcaggctggagacttctggaagccaacagcatcgctgtagaatttacagggtccagttcccggtggaccacaaaacctaaattatgtggctggggaaagctgaaatccaagggaagggtttgaggaggggctgaccttataataaaaccggcttgtatttactaagtgttaactatgcgctaggccctcgttgacgcctcaactctatgtgaaaagcactattatcccccatttacagatgggaaaacagagatttagagcgcgaaaatcatttccccaaggcgcacagactccaaagcccacgctaccaggtacaacctcaaggctgcggcgtctcttcacctgccccctagcccccaaaccgctgctatgtctagggcctgacattccggcgccctctgggacgtgctcagatgcaggggcgcaaacgccaaaggagaccaggctgtaggaagagaagggcagagcgccggacagctcggcccgctccccgtcctttggggccgcggctggggaactacaaggcccagcaggcagctgcagggggcggaggcggaggagggaccagcgcgggtgggagtgagagagcgagccctcgcgccccgccggcgcatagcgctcggagcgctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcagggcgcacggtacccgtgctgcagtcgggcacgccg

SEQ ID NO: 28 - DNA, 2500 bp, Homo sapiens
ccccccctcctcaaaactgaagagtttcctgttgtggtcttgtgggtttcagtcccttatatatgagccctctttagggaataaggagcttttgatagattggtgtcttttcatttctataaaaaatacattctgcaaactgtgcctggacatgaaagtaggtgttcttatttgtcattttcagcggaaagcttcctttccattcaacatgcataaagctattgatgaatgggggatagggacagcggcttcctgcaccattaatttatgtgagaatgtgttaacagcattttcaatggctgatggatgcggtgtaggatgggaaaggaactgtcctgagacactcgagtttcccattggtggggaatataaagatgtaagtggcaacctgcgagcaggcagacacgttcagctgcatcccttttctgagaacttgaaaacatactgctatcagaacaacccgtgtgggagtgaatggctaagtgtcaacctgggctgaggtgtccgttaatgtataggctctggggctaatccaggttagaggacttgaccaaaaacataccctcccaccaagcatatagagggataattttgcttaagaaccagtttttattttgttgctatcaaatttccaaaatacaatgttatagccttttgcctgaggcagttaaatggatttcttacttgtgtggcttttaaataacttgcataaacgttttctgtgctatatttgtgaataaaaatgattttctaatcatgtagcaccttgaaaaatatttttgtaaatgttatgaagggtgcaatgttagtgacacaggtgcaaatatgtttctaaatccaggaaaagtctgttttccaagtcaggtttgttctcaatgcttcatctatcttcaggcctcaaaaggattctaaagcatttctgggtttagtgaccttttctaaagaaaaataattaaggttttaatctatatggtcttgaacttaagcacggctgctcaggagctggatgaataaggagggagaaaaagcatatttaaaataaattattccgaagacacggcttagtgaaaaggtaaatggacataggaactttaaccagttacagcttggtgtgcagttgaagaatcagattttgcatttatgatttttaatctaagattcagagggaggaattactttgttcaaggtctatttatggtagagttgtctttagactcctttgaatttctttttattaatccaaacatatattattaaacactttttttttttgatgcaattttggctttttctgagagagccacattcaaagtcaggctgtcctagtagccagctgtgtggtcctaggcaaattacttaacctctctgatcctgtttccttatcagaaactgggaatagtactctgcagggtggttgggcagatgaaatagaatcacctatgtagtgtgaccctcccatagtagggcctccattaatattcagttttttcctctcatttctattgtttagaaggacagaaacatgcaatgtcctgcgaacatttccaagtggcttcctctcctccaaaagcttcagctgcctggcaaccctccccgcagtatcaagcaggcgcattttaaagtctctttagcgggagggcagggttagagccttcggtcatttgttttctaacttgacataaaacaacgtaacgcagtcgctcagcgagccgttggccagcctcgctcgcccggggaggagggaggaggtggcgcggggcgcgggaccaggccttattcggaggctgtcgctgcatccctacggccgcatcgcggccgagccctcgcctcccgggccggtgtctccggctgctcggaggcgtcgggagccgcgcgggactcggggcggcccgggcgcgcggcgctgattggcagagagggcgccgccgtccaggaaacggctcgggtttcagtgggggcgtgacccgccgcgaggaggcgggggcggcggcggcggcgcgggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaaggta

SEQ ID NO: 29 - DNA, 2500 bp, Homo sapiens
tatgtgttcacagatgcacggatgctcccctcccagagacccgtggccgtgggtgcagagggagtcagtccttcttggaggtactgggaaaggagcctacagctccactgagcccttctgcctacaggaggggccccactcagcagctgttccctgagggagacctgtccacgcgtgagctccttcccacagtcccatccagtcccccaggggaaggggcaagacggtcaggtcaggacccacacgcatgctggtgctttcagaacctgatgacttagttggttaagagagctcacacctgcaatcccagcactttgggaggctgaggcaggtggatcagttgaggtcaggagttcaagaccagcctggccaacatggtgaagccccgtctctactaacaatacaaaaattagccgagtgtggtggtgcacacctgtaatcctagctacttgggaggctgagacaggagaatcacttcaatcagggaggcagagattgcttgagctgagactgtgccactgcactccagcctgggtgacagagcgagactccctctcaaaaaaaaaaaaaaaaaaaaaaaagagcagggccaatgtgctgctgtgccttggggaacggtgtcctgggagcggacctggctggcagtgacctggggctgtgcactggagggggctggacagtgcacttggaggtgggtgagcctgggccccaagacggtctggggacatgggcttggccagagcaggaaatgagggtggccaaggacaggtgctgagccaccgagtcggcacagaggggtgggcatgagggcttcctgggagcagggcccttccaagtccctgaggtcagcccagggacaccctggcctctgggagattcatggtggggctgtgcatgggctgggtggggtcagcaggcagccagtcactgaggggctttcagcgcccattttcctgatgagcacctccagtcctggaagccacaagccaggcacgcagcccacccggccggccctcccggccagctcactggctcccatgggccccggacctccttgggctttgcctgaaggtgggagcagatggccttttccttgtgagggtggacacagaagccaacagtcatgtggggtgaggggacccaggcacctccagcgtcccccgcagggcagcaccttcccctcctgggaacccacccctcttcagctggactctgagcccaccccgtggagcccagaacctcgggccagctcaggctctgccaccagctctctgtcatcctccctcagagccgggctgtgctccccaagccaggggtatccagcccccgctcttcatcctaggccgggcaggcagccagccccaggcctgtgtcctccttggggggcggggggtagggacccctcgcgcggggctctcaggcggcttagctgcgtgcggccccaggtcagtcaacgcagcctctgcacctcgccggtcaggtacgcaggccgcgccgggctgccccagacctgcggcgcacacgggaggctcagcctggaatgccgtcctgccctcctccacctgaggaattgccgctcacaccacagggttcagctccgcggcatctcggcgaagcctcctgacatcccgacccccgccgagacctcccctctgagctcctgagcacagccccaggggacccgagctgcgcgctatgcaaacacaggcctgccctcgccctcggtgcgccccgtgcccgccgccccctgggccgccccgcggtgcgactaggggcctagcccgcctgccccggctcccacgccctcgagaccgccgggcgcccccgcccggccacgccccctcggatgccccgctccgccccgagggggcgtgccctgcgcccccgcgagccgggcggggaccgggcgggagcggggcggggccggggcggggcgcggaccgtcccccactggctgcggcgcgcggggcagcccaggctcagtgcgcggtggcggcggcgtcgcgggcagctggcgccgcgcggtcctgctctgccggtcgcacggacgcaccggcgggccgccggccggagggacggggcgggagctgggcccgcggacagcgagccggagcgggagccgcgcgtagcgagccgggctccggcgctcgccaggtccgtgcttggggccgggcaggctcgcggaggggtccaacggtgctcgcggaggggtcggggcgcggctgtcgcggaaccacagaggtccctgcagcgggccgggccggggcgcgggcttcccgctccggaaagtttgccgccgccgccgccctgggagggctttgcagcagccagggagggaagggggaggagggagggaccgcggcggggaggaggcggccggcgccaggcggcccgggagccctgggcggcggcggcggcgggcggggcggctgggggtcggaggggtcgggacgcaggagccggccaccgccgctttcgtccctgtccgcccctctaacga

SEQ ID NO: 30 - DNA, 2500 bp, Homo sapiens
cctaagcctcccaaagtgctgagattacaggcgtgagccaccgtgcccagcctaatatatatttttaatagagacgagatctcactatgttgcccaggctggcctcgaactcctgagttcaagcgatcctcccgattcagcctcccacagtgctgggattataggcatgagccaccatgcctggcctcaattattttctgatttatctttgggaggagaccattctttcgcttaagaagaaaaagaaaaaaacaattttaagtctctttaatgggccttacagttatgcaaagatatgccactatccctttgggagtgagggtggaaagaactagacttgcctgcgtaacaatgagaaaggaagttgcttcttaagacagggtctacactagctagaggggacaaccaggacctgagcctcagacagggtctcgctccatagcctgttgcccaggctacagtgcagtggagcgatcacggctcactgctgcctcgacttcctgggctcaagcaatcctctggcctcagcctcccaagtagctgggaccacaggtgcacccagcccatttcccttcctctcaaggtgccttaagagaaggagaaggcttgagattcaggtgagggatgggcagagatcagaggctgcctggcctcctggtttggccacagtctagatgctcaggagagaagtgcttacaggtgaggcaaacatgagccaggtgggcacggagctatctgggagcacctactgtatgccagacacttgaactctaatggtgaggtctgaggacacagttcctctactgaaaaatagagacagggcaactatgttgccagggctggcctcacactcctgaattcaagctgtcctcctacctcagtctcctagtaactagaactacaggcacccgtcattgtgcccggccattccacagctatttacataattaactgttcagttacaattctggcaagcgtgctgaagaaataacaggtcctaaataattacagctagtattacaaagcaagcgctgcatgccaggcttacacatactgctaacatgtctactttttgcaacaacctcttgtgggtgagactatcatttgcctgttttactgaaaggacagtgaagactgaggccttgtgtgatggtgctgagagctgggcctatgacgtccagccctgtagtctgtgctctgaaccacttcaccctgtttctttctccatcctcacagtagagacgtcatctaagaggagcttgcaagtccagagggcttcctgaaggaagtgagctgagaataatatgaaattaaccagaccaagggccattccaggaagagagaatggtacgtgaaactgcccaggagtagcagggtgggagccaggaggcgtgggtcatggcaatctatgtataaagagctatgggaatctataggaaaatttaagggagtggctgacacagtcagatttgtgttgtaagtatcactccgcaggttgcgcagggaacaaatgagggagcagaaggaaggggttctcctatccgctgcacggcactgcgcagaagacaggggagccaggcattccctgaagggtgaaaagcaaggagtagagctgggtagtagactagaatttaggagcctggcctggggcctgggtggggcgaaagaggcggagcctgaatggggtgtgtataggggggttgcgtgtaggggtgtgtgtataggctggggcggggtcccgggagtgggctgactgggtcgggggcggggctctccaggtgggcggggatcttggccacccctggccacacctctctccggctcgagctggtctaggcggggcgggcccgagggggtgtggcaggaggtgggcgggcccgggtggggggggggggggcgtggaaggaggggcgggcccgagcaggagggggcgggcccgaggggcggggtgggacaggaggtgggccgctcgcggccacgccgccgtcgcgggtacattcctcgctcccggccgaggagcgctcgggctgtctgcggaccctgccgcgtgcaggggtcgcggccggctggagctgggagtgaggcggcggaggagccaggtgaggaggagccaggtgagcaggaccctgtgctgggcgcggagtcacgcaggctcgaggtgagccggaacccttgtgggcccgggctgcgctcccagccgccagggggcgagaggcggcggggctacggggactgcccctcccggcgcaggggacctgggcgtccgccgggcggcagggggtggagggggcggtaaatcagtaacccgcagtgcacacagggccttttgtcccgctccgtccaaagagcaccccggccgcggagctggttactcattgcccaccgaggcgggggcaggctggccctgtgcagctaccctcgggacccattgattcgcacctccccccaggctggcccggcaagggtgggggaggacaagcgcgcttgtccctgcggctgt

SEQ ID NO: 31 - DNA, 2500 bp, Homo sapiens
cacaatgtagattcattgctggcagcaggtttggaaagtgttgtctctttatgagttaaaaatatcaaaatggcatttgaaaaaatgtattagcaaattaaacctttgtacatggtactatggaccactttcccccagagtcaaatgtgtgtctctcctgagaagccctccttgggttttccctgccaataaatgtgcctttacttctctgagctgatggaggactaaacacccctccagcactggaatgccggtcctgcagccacttcaagtgtccaccttcttatcacagtccaagccctttagaaggcaaagatgatggctctccatcagtgttttcagccctaacccaatgcctggcccctgttgaatgctgcagaaggacaatgctgacagcttctccttgaaagagggtgtgggagtgacaagcagctctcaggtgagaccaacagacactgtggctccgcacgtatgtcccaggctgtgttctgggctgggggaccagcaaggagaaggatcagctgtccgcttccagctgttctgagggatgatgctgtgggttttagaggaggcaggggtgcgggaggcagagaaaggcgccaggaagagtggtgaggagaactggaaaaacagaaagcgctggtgaagccagcaggcaagcaatgactccggcagtcttccttgcctctgaagcagaagggatccagggacagttagtggctcaggtcctgcggctccaggtgcccagacgtgcatttcctgtgtggagtggccggggcctctccttggtgtgcagcccagaaatgtggcaactttgggttacccaaccttcctaggcggggaggtagtccagtccttcaggaagagtctctggctccgttcaagagccatcacagtcccttgtattacatccctctgacgggttccaataggactatttttcaaatctgcggtatttacagagacaagactgggctgctccgtgcagccaggacgacttcagcctttgaggtaatggagacataattgaggaacaacgtggaattagtgtcatagcaaatgatctagggcctcaagttaatttcagccggttgtggtcagagtcactcatcttgagtagcaagctgccaccagaaagatttctttttcgagcatttagggaataaagttcaagtgccctgcgcttccaagttgcaggagcagtttcacgcctcagctttttaaaggtatcataatgttattccttgttttgcttctaggaagcagaagactgaggaaatgacttgggcgggtgcatcaatgcggccgaaaaagacacggacacgctcccctgggacctgagctggttcgcagtcttcccaaaggtgccaagcaagcgtcagttcccctcaggcgctccaggttcagtgccttgtgccgagggtctccggtgccttcctagacttctcgggacagtctgaaggggtcaggagcggcgggacagcgcgggaagagcaggcaaggggagacagccggactgcgcctcagtcctccgtgccaagaacaccgtcgcggaggcgcggccagcttcccttggatcggactttccgcccctagggccaggcggcggagcttcagccttgtcccttccccagtttcgggcggcccccagagctgagtaagccgggtggagggagtctgcaaggatttcctgagcgcgatgggcaggaggaggggcaagggcaagagggcgcggagcaaagaccctgaacctgccggggccgcgctcccgggcccgcgtcgccagcacctccccacgcgcgctcggccccgggccacccgccctcgtcggcccccgcccctctccgtagccgcagggaagcgagcctgggaggaagaagagggtaggtggggaggcggatgaggggtgggggaccccttgacgtcaccagaaggaggtgccggggtaggaagtgggctggggaaaggttataaatcgcccccgccctcggctgctcttcatcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacgagaggacggactctggcggccgggtcgttggccgcggggagcgcgggcaccgggcgagcaggccgcgtcgcgctcaccatggtcagctactgggacaccggggtcctgctgtgcgcgctgctcagctgtctgcttctcacaggtgaggcgcggctgggggccggggcctgaggcgggctgcgatggggcggccggagggcagagcctccgaggccagggcggggtgcacgcggggagacgaggctgtagcccggagaagctggctacggcgagaacctgggacactagttgca

SEQ ID NO: 32 - DNA, 2500 bp, Homo sapiens
atcccagcattctgggaggctgaggcaggtggatcacttgagcccaggagttcaagaccagcttgggcaacgtggcgaaaacccagctatgcaaaaaagtcactgggtgtggtggcgcacatctgtggtcccagctacttgggaggctgaggtgggaggatcatttgagcccggggggtgctgaggctgcggtgagcagatatcatgtcactgcactccagcctgggtgacaaagtgagaccctgtctaaaacaacaacaacaacaaaaaaaaaacaagtatattctgggcggggcacagtggctcatgcctataatcccagcactttgggaggctgaggttggcagatcaggaagtcaggagtttgagaccagcctggtcaacatggttaaacccccatctctactaaaaatacaaaaattagccgggcatggtgccgggtgcctggaatcacagctactcaggaggctgaggcgggagaattgcttgaccccaggaggcagaggttgcattgcagtgcagtgagatcgtcccactgcactccagcctgggcaacaaaagagagactttgagaaaaaaaaaaaaaagtacattctgaaaaaaaaagcaaaacatatattttgatgataaaatgataaacaaaagcagggaaatgataatgccagggtagtgggcatcttctggggttggtgaggggtttgtgatgggaagtgaggggatgtggggatgtcattctacatcccttggcctggagaatggaaacacaggtatttgttgctttaaaattcaggtttataatatgctgatatattatgtactttccataagtgtgttacatctcataatctttcaaagctaaaaaaaatcaatatatgattgataaatacctaacttcatgtatcgcttggttacctagtttccccaaccttatttgttaaattatctacactatccgcactgatttgtgattccagcttaataatatagccacttcttacctggattggagagagctggtagtggttattgaaaatctactcttgctttttggcgaggatgaaagggctttggggggcgggggtggggggatggaaaatgtgaatgaaacgaaaagcagggaaggaggttgagacagtcatggtcatgggcttcattattaaacaggaaaattctagagacaaactgaaaagagaacggaagtctgtaagagggttgccaagtcagaaagcaaaagcaaagtgtcgctttattaaaaaggtgggggcgggtcctgattgagctgagaacaaaacatctgggggagttctaaagctaggaggccaggcctgcagggagggggtcggtcatcccgggtgtcccgctcacgggtgcggcttccgtcgacactgtccgtcgacactgtgcaggtaaacactgctgaacgggttttgtttttgcttttttttttttttttttggctcccagactggagtgcagtggcgcgatctcggctcactgcaacctccttctcccgggttcaagcgattctcctgcctcagcccccccgagaagctgggattacaggcgcgcacaaccaagctcggcgtattattttatttatttgcttgtttatttttgagactgggtctccctatgttacgccggctggtgccactattttcaaagatgtaacatactatttttacaaggattgctttcttgtttagtctagattttaccgatggaaagaaaggacggaaaagagcgcgtgccgcattttgctcccctttgcccaccaggctccatcacctccggagcgcatcgcgagaccgtctgcgttacccgcgtccccgcgcccggagccgcccaggagggggcccggaccctgtggtgcggagcgcgcactgctcgggtgcaccggacgccccgcggcgggctgggagggacggggggaggggggcgcggggagcggcctgaatcccgggcccgcctccggcccccgccccgccccgccccgccccaggccagccggcgcccgcgcggacactttcagccccgagccgcggccgctcgggtcggacccacgcgcagcggccggagatgcagcggggcgccgcgctgtgcctgcgactgtggctctgcctgggactcctggacggtgagcgcggcgaacgggccacccgcccgagcgtggggctggccggggagggggtaccgcgcacctggggcgggcgctgagaccgctccgccgggtacgggcgggggacacggtggccctggtgcgcggacgcctgaggggagcacggcgcttggggcgcgggacctcgggatgaggaaggggcgggcggggaccgcagccgggtcagggccagggcggccgggccgcgcctcggcgcaggggtagctctcccggcccctgaatccgcgtctccgtcggtggctccgcgtcctcggagccgggccctgccgggctgcgcgctgagcccggatccttcgcgcccttactcgcttccccgcgggg

SEQ ID NO: 33 - DNA, 2500 bp, Homo sapiens
gaaaagaatactttacaagaatatttaaacatcaacattcatctatagacctaacaaaacataacagaaataaacacaattttttttgctttaaaaatcctattataaaacagtcaatgacaacttttctttcatgtaacttattctgaattagcatcatggtaatgttttttataattgtagttgataaataattttacaaaaataaattgttatgtaaattaaatgaagcatacttttttgaaaagaagacatgtattttcttacgataaaaagaataaatcattttcatgaatatgttctaagtccaatgaaataatttttagttaagacaaaacaatagatttatttaaagaacaatataaatacaagaccaaaacaacatttataggttgctgaacctgaagactcgaaagtcaggttacaatttctacttccagaagattgtttttcccctttcttaatttaaagaaaaatgtactcttggaaataggatagccaacaagtaaaagcaaaattgttccttaaaattgcaacatttgggcccagcgcggcggctcacgcctgtgatcccagcactttgggaggcggaggcctgtaatcccagccacccaggaggctgaggcaggagaatcacttgaagccggaaggcggaggttgcggtgagcagagatggcgccactgcactctagtttgggcaacaagagcgaaactccgtctcagaaaagaaaaaaaaaaacctgcaacatttcaaaagatgttatattagtcatttcagtgaaagtttaattagggaacggtgtctaccagaatatatttggcatccttcttttttctggtatttgcctgagtgagaaaagtgttaggaataggaaggccagatgagtttgctttccctccagagaatactggccaagaaatttcctgtccaacctcaggagcatcactgaagcctgtttatgaggaattgtcctaccacaacactgtgttatggcatttgggaagccctcagaggagctgcctgctctaagattgaggccacatgactcaggccattataaaccctaatgcagagccctgaagagggagagcatttggaaggggatagggaagcacataaagatgatatgctcagctttactgtcgttgggcagaaaactctctagtgggcttgagagggggaactgattttaaggagggctgaatagtttgggaacctacttaatgcaaccatagtctgtatttttaaaatcctcacatttcactacagatttgggttttagtgtcttggcataaatagggccttaatataaattcttagttttctggagacatggtagaggtcagatatcaagatttcataggcaaacaaaacatgctatatatgtaaaaaatgtatatacacacacagccttttgctaaactgaaagaaagtaaaaattcaaagcagtcaaggggttgaaagatggattgggtatggacttttcttcatctatgtcaatttttgaaatctctctcccatctccaccattccctttgttaattgtccttgcttacatttaaaggaagggaattagatcatcaaacaggtcactcacacctacctctaacttcacacaccttggtcaatgtgaaacaagcttgaactggttatatgcttccccaccatttttatttattgtaagctactttaaaaattttagacaatgttgtaataaaaatatacacctttttcccccattatccctggtcttctggtgaagcaagcagtgtaatcaatgattcttatgtgtgaggagcaactccaagtagaggcgttcatgtccctaatgcttttttatttttcctcatcagcaaatttaaactacaactttatcctgatcccactgctgctctttgcctcttctccccccagcccttccacatttaccagcctcctccctacctcctcccagcctttccctttgccctcccctttcttctagcccctcctcccagttgagtcagtggcttgaaacttttaaaagctctgtgctccaagttacaaaaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtaagtttttaaaaaatgagaacacgttacctattagtttctttttctacaagtaaaacttttctaaagttttaaaatgtgttaaatgtaattttgaattatgatactgtaaatggattgatggatttgaaagtgtttctatagttttaaagttaaaatttattgtaccaaatatatattttgactgttaaaaaagtttctattaacattgcaacaggtgaaaaggttcaaacctttccgattagaataaaggattagtttactgg

SEQ ID NO: 34 - DNA, 2500 bp, Homo sapiens
agcgctcagggcgggcggctggtccgggaggccacgccagcgcgacccagccgagtcggcccccagcccgggcccccacatttcctcccccggagggagggaggcgactctccgcgggctgccctccccagcgcccgccgcgccctctggcggccgccgcggggacgcgcccggggcacgcggcgctgcctgtctgggccccccttccggggcgcggggcccgcgaggggcggcggggtcctctctcctcgagccactctgggccgagccacacgggccgcgcccttccccctccgctccccctgagcccccaaactccgggctccacggtcgcaacgccgcgggcaccccagcctggcgtgaaagtgcccggcgtagtagcctggggggggggtcccctttctcccaggtgcgccccttccgccacgttcgggctttccagtacgcagcgaaaaaaatgccgcatgcacgcatttatttattttgcaacagctgcaagaaacaatgaagcttttcaagaaccggggaaacgcgctttccagccgcgctgttgttgttttcaatgaacctctcccagccccgcactccccgcccacccctcccctctcctgcccacccctcccctgcctagcctttccctggctacccacccctgccccgccgagaccggaccggcggcgggggcattgtttttggagtcgggcgggaggggagggcgcgtgcggggtggccggcgcagtgcggtgggggcgggagcgggtgggcacgcgcgcgtgtctctgtgtgcgcgcgggaggcggtggggcgggagatgggggcggcgcctcgcagtctcgcgccccacgcccgggctccgctccgcacgtcttggggaaccgggctccggttttttgcgcgcgccggcctgggccgggccctcggcgcgccgctgctgcggcggtggccgctcgagtgtgcgagcgggcgcgtgtgcgcgggccagggcgcgcgcgcgcgcgcgagcccccagtgtgtggcagcggcggcggcggcgcggcgaggctggggctcttgtttaccagcattaactccgctgagcggaaaaaaaaagggaaaaaacccgaggaggagcgagcgcaccaggcgaactcgagagaggcgggagagcgagagggacgccgccagcgagcctgcccacggccggcgctcgcagaccctcggccccgctccccggatccccccgcgccctccacgcccctcccgcgcgggggcagctccacggcgcgcctcgcctcggctgtgaccttcagcgagccggagcccccgcgcagagcaggcggcggcgggcgggggccgggcgggggccggcgcggggcgggcggcggcgcagagccgggcggcgcggcgggagtgctgagcgcggcgcggccggcccgccgctttgtgtgtgtcctggatttgggaaggagctcgccgcggcggcggcggcgctgagggaggaggcggcggcgagcggagccaggaggaggaggaggagggggagccgctcattcattttgactccgcgtttctgcccctcgccggcctcgcctgtgacccggacttcggggcgatcttgcgaactgcgtcgcgccctcccgcggcggaagctcgggcgtccggccgcctcccgcgcggccagggccgggcttgtttttcctcgcctaggcagatttgggctttgccccctttctttgcagttttccccccttcctgcctctccgggtttgaaaatggaggccgacgacgccgacagcccgccccggcgcgcctcgggttcccgactccgccgagccctgggccgctgctgccggcgctgaggggccgccccgcgccgcccgccccgtccgcgcacccggagggccccggcggcgccgccttcggagtattgtttccttcgcccttgtttttggagggggagcgaagactgagtttgagacttgtttcctttcatttcctttttttcttttcttttcttttttttttttttttttttttttgagaaaggggaatttcatcccaaataaaaggaatgaagtctggctccggaggagggtccccgacctcgctgtgggggctcctgtttctctccgccgcgctctcgctctggccgacgagtggagaaagtgagtatgtgcccgccgcccgcggccactgcgggaacttttcctccgaggggctgcgccctgtttgcgaaacccgagttgccaccgtcgcagctgtcgggcccccgggctcgggagcggcggggtggggcgcagggcggctctgccgggagggaggctgcagcggacccgggacccgggctcgttcgtctggagccccgcgggccctggctccgcgcatccccgcgcgggaagggcttttctctttctccgtcctgacagcccagccgtgtttcccggctaggcgcgaggactcggtagggaagttggtgccggggcgggctccgcggttcccgggccccgcgacccgcacctcg

SEQ ID NO: 35 - DNA, 2500 bp, Homo sapiens
agaggaagtgtcatgaactgtaaataaggctgatagggaattgtttggtgatggggatggagaaaaactgccctctccaaaccaggttccatctcccacccctttatccaactttgccagagtccatcctattttatttgctgagtttactagcaggaagagaggagtttctttaaagattctctcccctccccaaataaatacctcccagataaatatgagtacatctatatagtagagcactatgccaacattgaaaagatggaaggatggagctttgttcgcagccagcttaggcaacatagtgagaccctgcctctatataaaaataaataaataaataaataaataaataaataaataaaagatggagctttaaaggttgatgtagaacgaatctccagtgtatattataaagtgaaggctgggcagggtggctcgtgcttgtaatcccagcactttgggagactgaggcaggaggatggactgagtccaggcattggagaccagcctgggcaacatagtgagaccttgtctcaaataaataaatcaaatttaaagggaaaaaagccaaggctcaaatagcatttataccaagcaattgtgggaagagaagggtgacatgtctacatacttgtgtttaagaatctgcttggaaaaaaaaggaaacataaaaatgcctctgccaaaagaaaagtcttttcactctaagcctttttgtgtttttccaaaatttttttttgccatatacattcattatatttcagcattaaaatatttcacctgtcaacttggaaaagtaaaagcgtgaatacagtaaatatttaaaaacagatgaagataagtaacaggttacattaattgtatacaatcaatctagtttgaattaggtgtagatataataattctgtgtgtttatcttggtgaagaatggtcctttaggttgtcattaaaaagcacctctgggaggtaaaagacataggcatctggaagattcatatttaatgaagttaggggacatttgatgtcactccccaggtgtggggagatatggacagaaaaccagaaggaacgaatgtggtcaggaaggaattggagttcccctccaaacagctgccacaagaagtccacacacgtggagacagttgtgcaatttggtcacagagtgtcctaggtgcttttgcttcattgctcctccactgaagcacgctggcagctggcctccttcccctgggactaaggatatcttggctggaagctctgctctgaaaaggggcatggccaaactttcactagggctcttcgttggggagcacgatggacaaaagccttcttggggctaggcaggtcacttcaaacttggagccgccaaatattttgggaaatagcgggaatgttggcgaactgggcaagtgcgttttctgattaagagcaaccagattcagctttttaaactacaattatactggccaaacaaaatacccttatacaaaaaccaaaactactggcaggagtcgctgccagcttgcgacccggcatacttggctgagtatccgcttctcccttgtggctccaaactgctgcagattctcggccacttcagacgcgcgcgatggcgaagagggtcctgcactttgacgcgcctggtgagggagcgctgctcttcgcagcgctcctggtgatgctccccaaatttcggggaccggcaagcgattaaatcttggagttgctcagcgcccgttaccgagtactttttatttacaccagaaacaaagttgttgctctgggatgttctctcctgggcgacttggggcccagcgcagtccagttgtgtggggaaatggggagatgtaaatgggcttggggagctggagatcgccgccgggtacccgggtgaggggcggggctggccgcacgggagagcccctcctccgctccggccccgccccgcatggccccgcctccgcgctctagagtttcggcaccagctcccaccctgcactgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtaaggagcccactctggaggaggaaggcagacaggtcgggtgagggcggagaggacctgaaagccagatctaactcggaatcgtagagctggagagttggacaggacttgacattttgcgatctttcatt

SEQ ID NO: 36 - DNA, 2500 bp, Homo sapiens
gtatttatcatctattttattttccagtttgcattggcttttctgggtctttctacaactttaattgtaaattttttcttttatatttttatcatggcactcaaactcagaaaacctgttaaggcaactacataatgtgatctaaaaatccagttaacatattgctatggaaaccaggaaatagaaacagcactctaatcattcctttagacaactatggctctgtgtgtgtgtgtgtgtgtgtgtgtgtgtgtgtgtaaaactttttaaattcattaatttattcatcttttattgaaagctgttactgaacccaaaatctgtaattgatctgtccataaataggcccagtgccttattaccagtaaattagtgttttttaatgagctatagtcattgataaattatgccatgccgtatcaggaaatctccagtcaggccgcgttgtttatttagcctgctgaggcctcttggtatggtgtgaaggacacctgactgggctgaaagctaagttctaactttgcccctcttactaaccagctatgtgactctcctgggaacttttagggactcagtttctttacctgcaaaatggttcaatgcaagactttagtaacgtaatgggaactttccttttccataaaactggggaatcaagaggtaatctcttttgaggactgaaatcactcttatgtaacctctggttacattatcatttccaagtgcctggcacttgggaaatgataactattcttactacatttttctatgtttcattctgtagtaaataagaactgaacctgcatagtaactgttattttaacccatgactttcaataacgaagatatctatgtctcattatctattgccatgattgaacaagttggtatgagagccggaacgaactcaagttctaaccggcaatgcccgttccttagatcctattacctttgagtgttcatttactcttgtaggtgccaatttttatagcgaaatacaaagttatcccaacacaattactcctaatagagttcaccgaggccccaaaagctcttttttaaaaatcatcataagatttcaacattcaagaattaaacttttgttctgttgtgcttattcatcgctatttgcccagttatttaatcagcctgcttccggctatggaaaaaaaaaaaaagaaaaaaagaaatggaagtctcctcagggttaaactcctctgttgttcttccttgcagaaatttgagttattatagtagaggataatcgttgcataatgaaatcattgggacaattcgtccatccacttctacctccgcctctaacaatgaactccttgtttctgcggtgcccaaatctctctaaacccgggtgggcgcggggcggttagcggagacgtgggagaggccgagagcaaagctcgcgcccttcccggggtcagcgagcggggtgccaggagggtgcgcgccctgcatctgagcccggggtgacactcgcctcccaagcgccaggagggggagactcggtcccgcttatctccggctgtgctaacttcagactgcctgagctgggggaggagagcgcgcagccagggcgagaaaacttctccacctagaaagtttcaccttgtcgtgggcggggcagaggcgggaggaaacgcgacccccgcggggccaggcgcggcgcggacggcaggaagggcgggggccgatttccctctgggtggtgccagtccccacctcagcggtcctcggaacccgcggactaggggacggacagcacgcgaggcagacagacacgtgctggggcgggcaggcgagcgcctcagtctggtcgcctggcggtgcctccggccccaacgcgcccgggccgccgcgggccgcgcgcgccgatgcccggctgagtcactggcagggcagcgcgcgtgtgggaaggggcggagggagtgcggccggcgggcgggcggggcgctgggctcagcccggccgcaggtgacccggaggccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaaggtaaaattgcagcccctttcagatccagtacccaatccctcgcctcaggggttctgctttctttgttcccctaagagacctgactgctgttccagggggcaaaaccacgtaggtgggctagagtttaggggcttcggaaactgaagagacgtggccacggcgaggacgaaactagaatggggcttgtctttttagggggttgcttctgatggccacctgtatgacttaggagggagaggggcgctgggacagtgggtgatgtgtgactgttacggcccagcaagttttaaagctgggatctgactcagcccttacaaaagggatccg

SEQ ID NO: 37 - DNA, 2500 bp, Homo sapiens
gtttattttgcttttgcagaggaggcagatgttgacctctcttctttgcacagtctctccgcaaaattttcccccaaccatttgctttcttgcgccaccccccaccggggcactatccctggagtcagctcctcaggcctttaaaccttccagaatgtcacacatggaaacctttagcaaatgtttgttaatgatcataacaaaggcatcattcaaattaggcaggtaattactaccagaaggacaactgggtgctcgcttgctcatccattgctttctgctttaaactcccgaggagtcgctgcctggagacactcccgcccggactgcttctcgggccccattaaacggctctgaaatgaactgtgaagttaccatttgtgggcggagagcgctggggccctgcggacggaagaccctggcttgaccggactaacagtgtcgccgaagcaaagcagcccctctgggtaggctgtctgtctcgcctcctaccctctggggcaactagcctaaaaacccggttctcaacttaacagcatgtggcccagcacccgcttttgccttttattccgcactctgatttttggggggatcgtttgttcccgctcattttctacaaacattacggtatcctgttagcattccgaacaaggggctgttcatatatttgccttcaatgatttcctgaagggacatgtggaagtaatagcgagcggtgcagtcacccaagctgaagatgcacgagagcgggtcgcgtagaagaactgcggcaatgggacccccagcgcagccccaggcgccctgttttcgaggctgacatcccaagatcctgtcctggttaggcacagcaccgcgcccgttggagtacttctctgtgactctcccctgtccactgcactgtgtccaccctcctggtgaaggaggagacggctgttggacatgccagtcccaacctggccactttggccattttcagagtgcgtgtgttggggtggggttgagggagaaggatgaaggatgacccccaaccccaacaggagaattcaaaaatcagagagcgatgaaggtgaacgcgctcccctcccccgctgtcgctgggaagccccactctgcccaggtctggttcctggagcatcagcgccccctttccctcgccggcaggatcgtttcctcccgacactggcctctcgccttgggcgcagggtttggggctcagctgtcaattccaaggaattcgtagggttcgacccacgcggcgggagggaacggagggagaatcaaaaggcatcctcccccacgacgcggaggcggtggtacgattcctcaaacgcccaagcttgagctttttttgtatgcgagatagaagccagggcaacctcgcccagataaccccgaaaacaaaggcacgaccagataagtgacttcaagggagtaaaggtcgttctatcggagaacgttccaaacctacacagaccctcttttctttgtaccgtatttcagaccacccagtcttgtacacacacacacaccacacaaaacaaaaacccatcattgtcatattggactcaacagtttgcccaatcctattaaataactagcaactatatagcccttcccctaaaagacccctagttcaaaacggcgcaaccgcttggagatttctcccgagggccctatttctcgttgggccgagtttgtagaagggacatttcttgattctagatttatattctctctgggtattaaatgcaattttgtatgttccttttcttcttttcaaagagaatacaaaactatagcagtctaagaaaataaccccaaaatggggaggcaaactcattctggaaatgatgggctgtttgtagtttcacaaaacctctttcccggcagagcaccaacacctcccccttccaccaccccccatcccatctggtctgcttctccccgccccccagttgttgtcgaagtctgggggttgggactggaccccctgattgcgtaagagcaaaaagcgaaggcgcaatctggacactgggagattcggagcgcagggagtttgagagaaacttttattttgaagagaccaaggttgagggggggcttatttcctgacagctatttacttagagcaaatgattagttttagaaggatggactataacattgaatcaattacaaaacgcggtttttgagcccattactgttggagctacagggagagaaacagaggaggagactgcaagagatcattggaggccgtgggcacgctctttactccatgtgtgggacattcattgcggaataacatcggaggagaaggtaagggaaaagaaaaaatgattttttgtttataagggaagtccctgatcagactctgggactggagaaagcgaagtaacttcaaactttgggcgacaagaaaaaaaaaaaggagctggattccagttgtgtgtccagagagtaatttttttttttcggaataaacttcacttggggacgg

SEQ ID NO: 38 - DNA, 2500 bp, Homo sapiens
tttccctgttgccacaaagacctggccaggccccctctctcggggtactcttatggtccccaacccgtggctgggaaccagtctgagaacacttctccttctatgtggtccagcccagatgctctgctgggcaatcccgattgggactagaccaagaaatgcaagatagttgactgtggtgggcatggaatcccagctcacgcaaatcctctgctctgtgaacaggggtctgggggaagctgaggaggaaagacttagcggcgcagagggaaggagggcagagccagcctctacctgtgtcacctgggacagctttcttcaccctctggaattcagttgccctgtgggtgcagtagtgggagcaggggtggtaagcccactctatatgcccttctaaaataatgccctttgccatctgagggctaggagtctgtaactggagatgtcttctgatctgagaagctgagggtggaagaagagacccagctgggggaactggaggagtcttggcagaccctggtaggaaaggagagccagagaatggaaatggaacaaaacggatacttgaaagtatcttgttgaggctgggtgcggtggctcaagcctgtaatcccagcactttgggaggctgaggtgggtggatcatgaagtcaggggtttgagaccagcctggccaacatagtgaaacccggtctctacaaaaaatacaaaaatagccaggtgtggtggtgtgcgcctgtaatcccagctacttgggagtctgaggcaggagagtcacttgaacctgggaggcggaggttgcagtgagcctagactgcgccattgcactccagcctgggagacagagcaagactccatctcaaaaaaaaaaaaaaaaaaaaaaagaaagtatcttgttgagtgactgaatgaaagagccacagggggaacctaggaagaaagcagggcagggggttcatctacactggaataggggtctcgggtgggggtcagatgacttgtgtctcttccatcatgtgtctcatgagacctagttctgccccagctctgccattgctgcgtaagcttgggaagcttattttgctcctctgggcctcagtgtccccatctataaaatggggcaaataatcctctccttgcaccccaggggctttgagacgtgaaaaggaatgtatctgtcaatatccagagatatccaatctgtgctggatgaccttgaccaaatcacattccctctctgtgccaattcacccctgggccttggtttgtccttctgaaaaacggctgatcatcttggaggatctttccatctcctccatgtcacctcttctcagcagtgctctctgattctctcaggcttccgttaggggtccttctaaatgcactctcagcctcctgtctgccgaccgggcacctgcttgcccaatgaatatcttgctcactagcctggtgcttctgaggtcaggaccacgtcttgctactgtcccctgcacccagtacagtgttagaatgtagtaaatgctcagtatgcttgttcaatgaatgaaggttgactgaatcaaccacagagatgatcagagtctcctttacagatggggaaactgaggcactgagaaatgatctccctgggtgccatggcaaggggaggccgccacccgacatgggcatcgggaaagtggggccgaggccactctttgacaaaagtgactcagcagctggcggctggcgtgggctgtggaatttctaagcatcccctcacatcctgagcgaacgggcgatgggagggagtaagcagagtgaaaagaagaaaaacaagaaacttgggggaaaagaaagagagagggggaaaaaaactgaaattgaaaacagacgcacgcgtccaccctccctgccccgccgcccccccttctcccagctgggccaatcagaagccaccctgcagcctttcccccaaagtggcgggggaagtgggggggcatggaagcccatagtggtgtgagctcctgaggctgccagcagccagcagtgactgcccgccctatctgggacccaggatcgctctgtgagcaacttggagccagagaggagatcaacaaggaggaggagagagccggcccctcagccctgctgcccagcagcagcctgtgctcgccctgcccaacgcagacagccagacccagggcggcccctctggcggctctgctcctcccgaaggatgcttggggagtgaggcgaagctgggccgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggtaagtgctggaaaaagtgggaaccccagccccatgc

SEQ ID NO: 39 - DNA, 2500 bp, Homo sapiens
gcacagtggtgtgatatcggctcactgcaagctccgcctcccaggttcacaccattctcctgcctcagcctcgcaagtagctgggactacaggtgcctgccaccacgcacccagctaatttttttgtatttttagtagagatggggtttcaccgtattagccaggatggtctcaatctcctgatcttgtgatctgcccgccttggcctcccaaagtgctgggattacaggagtgagccactgtacccggcatttttattatttatttattattatttttgagacagagtttcactctgtcgcccaggctggagtgcagtgcggcgatctgggctcactgcaagctccgcctcccgagttcacaccattctcctgtctcagcctccggagtagctgggattacaggcgcccaccaccacacctggctattttttttttttgtatttttagtagagacagggtttcaccatggtagccaggatggtctcgatctcctgacctcgtgatccactcacctcaacctcccaaagtgctgggattacaggtgtgagccactgtgcctggcctatttttaaattttttgtagggatggaatcaagctatgttgcctaggctggtctcaaacttctggactcaagcaatccccccaccttggcctcccagagtgctgggattataggtgtgaaccactgtgcccagcctagaaggagtcgctttaagtcaggtatggtcacgtcctccctgcttgaccctctcccatggctgggcttcagctactctctcccctcactccccgacaatcttcgcctgtcctggtcggctgcctgggtctgctcatttacccttcaaaattcaactcagcatcacttcccacagggaagccctccagaaccccttaggtgccccacacgtggttacctacctcctctcactcatttttcttttttctttttttttttttttttgagaagaagtcatgctctgtcacctggccaggctggagtgcagtggcacaatctcagttcactgcaacctctgcctcccgggttcaagtgactctcttgcctcagcctcccgagtagctaggactacatgcgtgcactaccacacctggcaaatttttgtattttcagtagagacagggttttgccatgttggcctggctggtctcaaactcctgacctcaggtgatctgccctcctcggcctcccaaagtgctgggattacaggcatgggccactgtgcccggcctctttttaggacggagcctcgctctgccccccaggctggagtgcagtggtgcgatctcagctcactgcaacctccgcctcccaggttcaagtgattctcctgcctcagcctcccgagtagctgggattacaggcgtgtgccaccctgaccggctaatttttgtatttttggtagagacagcgtttcaccagccaggcggtctcaaactcccgactttctgtgatccgcccgtcttggcctccccaagtgctgggattacaggtgtgagcttccacacccggcctcctctcattttgagcttatgtaaatgcctaccggcccccagtagggctggggcagggttgactttccagtgctgtctgggatggctgggagcaaagaggctgaggactgggcagcatccccctagatggccccatcctgttctggccctgttctggcttccctaactcagcttgggttgggggcagccccaagccccatcccagaccctctgcagctctggcaacagctaggtccagagatgacctcagcctggggagccagtgttcctggggagacagaatctcattgcctcccaccccaacctgagaaagcctctcgtttgggggtggggtgaggttcagggagggatcctggatctactccaagccctgtttgctccactgaccaggagaaaaatgcggaaaaggggtgaaagggtgacatcacccaggattgctccacctctggggcaattatcgggagcagggcggccccagtcagacgcaggcagccccaaagcctgaacaggcagggccagacccaggtaagaagccaaggtccggaagggtcgcgggcgcggggcagggctgaggccccacgtcaggtcgcgtgggtctgcggagctggggtcccccacgaagggtttgcgggactcgcatccggccttggccttggatgtttatgggtctgtctgtgggtgagcctgtgggtcccggggctcccgaggcctgtgcggctttcaggtctgggggtgctgccggtgcgcctctgtccagggaccagctcctctgtcccgggtgggtgtgggaggcggacccggtggggaggggcggtggctgccctgaaaccagagctccctctcgccggtacccaggtctgtgtgaggcttccggcccccaggctcgggcctgcgcccagcccaccctacacccctaggcgtgtgcccccagccgtgcccagcaggggcggagggagggcagaagctt

SEQ ID NO: 40 - DNA, 2500 bp, Homo sapiens
ggaaaatgttaaggaactggaggtgtgattaacactgattaatctaggctccgtacgtcaaaggcacagactgacatgaacatacatgctgattagaatcatacagagctggaaggggatgggcaaagaaatggcattagctaaatatttactatgttccagacactgtaccagacacattacgtcatttaaggggcccaagacaagtgattcaatcccttttatttataaacggggtaggtactattattatttacattttagagatgaagaagctaatggccagagaaaaacagtcactttctgagatcattcattcattcatacaataaaaatctcattagctcttatcttatgaaagacattgcaaatagagcagcaaaaaggcagtctagggacctcaggtggcacaaagtccaccaaatctccattgaagaaactggaactgagagggcaaattgggactggagcctgccagaagccatctctctggaatccctcagcctgagcaagaggggagtttgctgctgaggctgctggagctgcggtgtagagcaagttgcaggcctctgagcctgttggaggttcagaaaggttagctgacttgcttaagtaagtgtcctagacaggagggagaaagcgcgcgggtctcagatcccagctccaatatccacgccttttcccttttcatctcccggaaggaagccttcacaggtggggaaaatgctcacccagtgactctaaagtagtgcacaaattggcattaatcagaaccattctgattcctgctatcaaggtttatttaattctaagctcaaatgacacagaaaataaaatttcctccttaaaaggttctgcggaaaaatattctctgttctacgtaacagcctccttggctgtgtttgtgtctgaagaaaggactagtctgacaggaagaattacaaatctggagctcattttggcgtcgcagagggaaggtgggtggagcgtctcgcagtaaattaggaattcagaatgaaaacgtagagtttatttgggtttttagtgacacctcaggattattatacagcagtgtcagacacagctcaaagtaatgatgactgctccccctccgccccgcgggagctgggtgctaagaagggggaacccctcctttgaggggttggggtggggatcctccctttccaacctggatcgggaaggggtttgagaggagtgcggcttggatccccatcctggatctgtgctggagggaggcgggaggctatttgggggtgtgtttgggctcaggatctgggtctgacgctgggatctgaggactttctgggtttggaactgggtgggggagctgggtttggggtgaccgctgggagcaggaggaaataggagagtgagtcgcatgtgaagggcactgaggagggatcttcgggggaatgctgaagttgatgcgtggacttggagaggggaagttgaagtggatgcaggggcctggcgctgcagcggatctgggtggcatgtgctgggattgccaatgcggagctgtgaatggggtcagggtggggctgaggcagtaccagatcttccgcgcctagaggaggttagaagaaaagagcgtcgaacggccacagcctttttccgaaagagactcacacagacacacccatcgctctacccggccctccggggctagcggctgagcggtgctggggctggcagaccccgcccccacgcggcctctcggagccgggagccgatcgggccggggctggggtctggcggcccagcccccggggagcgtggggcgtggccagaaaccggcgctcgcggctgcggattggctgcctggcgggcccgcaggcggggctcccggctgggtcccgcttggcagctcgcggcggcggggaggcggggccggcgggagcccggcagccaatggacgcgcgtcctccgagcagttacaaagggccggagcgaggccgccgcggcggctagggaggtggggcgaggcgaggtttgctggggtgaggcagcggcgcggccgggccgggccgggccacaggcggtggcggcgggaccatggaggcggcggtcgctgctccgcgtccccggctgctcctcctcgtgctggcggcggcggcggcggcggcggcggcgctgctcccgggggcgacgggtgagcggcggcgcggcgggcgggcgactgcggggcgcgcgggccggacccggcctctggctcgctcctgctctttctcaaacatggcgcggggccgggggcgcaggtggcggcgccggggcccgggccgggctctcgtggcgccgcgcggctcggcggctgccgggcgaaccgcaagcggggagcggaggcggggcaggggcgtgtgtccgggcgcgggcggacgtgtccggctgcccgaggccgccccgctcgggactccccggggaggtggagtcgggcggagtcgtccgaggtccggggctgccctctggggtgtgagtgggc

SEQ ID NO: 41 - DNA, 2500 bp, Homo sapiens
ggaaagatacataatacctgccctcaagaaatttggagttgagtggaggatagaaatataaattaaagaatgacacaaataattataaagttacagctgttaaaagaaaagcatatggtgccaagagaacgtgtaatacaagatctactcatggaggtgagggaaagcttgcccatcaaagaagttatgattcaatccacgaagaccaggagttggctgggtgaagaaaaaaaggtcagaggaaggaagtccacactggggaaggctctaagcataaagggtaggaggattacagaggcatattcacgaaatttggagaaggctttcagtaagcaaggagaagccaaatgaaagtttacgggagagttggaggcttgaagacacgttcaaggatctggtttttatcttctctttatctcaagagcagtgggaagccattaaatgattttaatcagagggttggtataactagttttgtattttgaaaagctgaattcagctctcgtttgagaaactgagtgaaagagcccagaacggccgtggctgagggtgactcgtgggagactcctacacaagccatggcagtggcatgggctggtggcagaagagggaatagggagaagatttggaactcaatcttcctccattgacaaagtcactccagctttggcaaggcaattaattggtgggaaagaagatgcctagccctcctgatttcactgcactttctgcatcttcaacatgagtactgggaagtggcaaaacatccagaggcagcttgggtgctaggtggagcatgagttaaaattccaggatgaagcaaatgaacacttagaatgacaggaaagatttgggagttgggtttgggggagggctatttacctttattccctggagaccctggcacaaacccttgcctctgcaatcttcctctcaggtaaaggaattcattaaatgaattgctagaagatctactgaccagagggctgtacagaatcatatctttgagagtgggaagtaggttgatcacatagtttattatccaatcaggacatatctgaaagagaaagggggttctattaatatttaaactacaaaacatgtacaccaggaatgtcttgggcaaatctggttgccctagcaagaaaggaaatttgaaagtttatactgttctgctcccatgttaccccgtttgcacatgagagggtaagtattctctttcttcacctgcattaagggaataaaagcacaagcattcaggtgactcccaacccacttttaattttacagtttctgctatactctatacattctgaaaattacatttcccaccactatcacttcgtgataggtgatcatttacaattactcactgactcagtcccgggaagaggcggtgcaaaatgggacgctctatccaggtgctcattagaaatgcagaatctctgcctgcctcctagacctactgaattagaatctgcatttttaaataagatttccaggtgatcaatatgtacattaaaacttgagaaaaacctctagacttcgacctaaagaaaaacattttacaacttgacagtgtatgcacatacatacatgcatatagacacaactgaagcacaaatttaatgaagtagaatttaccgttactattttatttgggaaagaaatgtgctcgcgactcaatagattggagtattcactcctggatctcaacttgcaatttgaaaacgcatctctaaagcacctaggagcaatctgaagaaagctgaggggaggcggcagatgttctgatctactagggaaaacgtggacgttttctgttgttactttgtgaactgtgtgcacttagtcattcttgagtaaatacttggagcgaggaactcctgagtggtgtgggagggcggtgaggggcagctgaaagtcggccaaagctctcggaggggctggtctaggaaacatgattggcagctacgagagagctaggggctggacgtcgaggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcgggtgagtggtccccagcccgggctc

SEQ ID NO: 42 - DNA, 2500 bp, Homo sapiens
aaagaaaggtttattggacttacagttccacatggctggggagacctcacaatcatggcagaaggtaaggaggagcaagtcacatatcacgtggatggcagcaggcagagagagctggtacgcggagactcctgtttttaaagccatcagatctcgtgagacttattcactatcatgagaacagcacaggaaaaactcatccccaagattcaattacctcccattgggttcctcctaggacacatggggattgtgggacttacaattcgagatgagatttgggtggggacacagccaaaccatatcaaatactgtttaatttgagtctaggagaaatcttaaggccttaaacttagactttataaaagcagaggctagctccatctggactgtttattccttcagaatattatgcctacttgtattcccaatgcctgactcagggctctcccatagtcagtgctcaatgaatattggtaaggaaaaaaatggttcagtgaggagatttttgcagtaattcaggtgactgatgacatttgacctttgtgaatgaatctccaattctgcaattgggtggattctctgcctataggtacaagaggaagaggacgagaaatatttcctgttctctcaataactttccaaataacatgtcataagaagcctcctcattctactctccaacatattaaaagcctaggattgagccaggtgcagcggctaacgcctctaatcccagcactttgggaggccaaggcaggcgaatctcttgagctcaggagtttgagaccagcctgggcaactaagtgagacccccgccctgccaaccccctctacaaaaaatacaaaaagttagctgggcgtggtggcatttgtcagtaatcccagctactcaggaggctgaggtgggagaatcgcttgagcctgagaagcggaggttgtagcgagctgagattgagattgtgccactgcactccagcctgggcgacagggcgagactccatctcaaaaaaaaaaaaaaaaaaaaaaaaaaaaaaaaaaaaggcataggtccatagccctttagaactggaagaagccttctaggccaagcccttcatttgaactgagccttgacgggtattagccgttagccatttgtggaaggtcgcacagtagtcaggactggaagtctggtctttgaaacctgtcagtccacggtcatttcacctgcctgtttgtaaaacaatgtcctgttacttaagtatactttttaataagaaaaactgggtgtaagcaagagtatcagagtggtgttctggtgccagcagttcaacttctgcagcgtgtacttcacaggcacaggtacttaggacgtcgcacaggcacaggtacttaggacgttgccccgccgagactgtcgttcatatcgcgcccacctccgggtaacgccggtaatgctgacctagctccattatttgtattaatttatttagctagcagggggaacggctgggtaatgcagtctcagctgctggaggggaagaggcaagactccgccggcgcccaccatcagagcgtgacaacaagcgggaccggaggttgggaaaaagcacagggacagaatgaatgtgtgcatgggggagctctgctggggagagggcaagaggctgtgatcagattatctgaggtttcagaacttttatataaagaccaaggaaccaaatgaaaatccaactcttccagggagccctggaaagttcaaaggacctcgtttgagggagcgagtccccgcagctcgtgcccacgtctccctcgcgcttcccccaggacaggggccgccccaacccgggcacgctgagcccggccgcgcccctagccccttctctctcggcctcctcgtcccgaagggaagcacaggctcgagcagcattcggggctcatccatcagccgcggaggcaaagggggaggagagaggaggcgggaggcgggaggcgggaggaggagtttctgggcggccgagagcccccggtggggccggcggaggagcgcccttccccccgcgcgctgattgctgtggcgtcctgcccgtccccgcccgcgtgtgtgcgagggagggcgagtgcgccgggtcggcctgatgggggtaatcgagggtttcggggacgccgagcggcactttcctcttcccagcgagtgaaggagggcagtcggcggctctcgcgccccggccactttccctgcgcgattcccggagctccctgcaggaggtgagagtccccagcgggtccggatggcgtagttttgccgcggcgcagcagctgccggagctcgccgccgccgagcgctgggcggggaaacttgccgccgctttcctccaacttgctgcgggtggatctccgctggacacaccgcctccgaggtacgtgccggcccagccccgcggcgctgcagcccgcccgggctgtgggctctgcaggctgcgcgggtgcccgtcggcgcggcgcgggctgccagtgc

SEQ ID NO: 43 - DNA, 2501 bp, Homo sapiens
tgcttgatttaagcatcaagctagctctgtgaagggtaccagcaggtttcccattttttagatgagcagaccgaggttcttctcgctgcttcatactggaaacttgcacttgattctgaggctcctgcttcttcaagaacactgctttgggttcgcttctcctgtccctggggtctccctttgtgatggtggtgagctgcttcctttctgaatccagcttcaaccctacagttctccagaagctggacgatggggtggagtaaagtcagctccccccgcagtgagggacactgaagctccattctcatctgcggatcacagaggggaagccaggaagagccaggggacggtggacttggggctgggaggtcatctcagagggataaggggtgaggagctctggtttcaagttccaaagccctaggacctccctcttctctgtctgcctgcatttctagcagcctcagcagctgcaggcccttgggcggggctggatgtagggaaggtcattgtaccaagaagatagttgggtaaatgtggtacctttgttgtaggattctcttgggagatgtctgcatcaatgaggatggcataaagtaaccagagtcaggatgtggggtctgactcagtgacagaaaaagtggcagtgtgtctctcatagccaaaggggcccttggaccggcagtcgggagtctggggttctctgttggctctgcctcctggcacattgggtttctggacctcagttttctcctctataaaaccgggcagttgggtgggcacggtggctcacacctgtaatcctagcactttaggaggctgaggtgggcagatcatttgggcccaggagttcaagacctgcctgtgtaacatggtgagaccctgtctctacaaaaaatacaaaaattacccaggcgtggtggtatgcacctatagtcccagctgcttgggaggctgaggtgggaggattacttgaacctgggaggtcgaggctgcagtgagctgcgatggtaccactgcactccagcctgggaaacggagcggaccctcaaaacaaaaacaaaaatgaaaaacaagcaaacgaagaaataaaaaaacctagggggttgtagtcgatgatctgtaaggtgagttataattgatgtattggaatatttaggaaaagggcactgggaatatgctaggaacacctgatggaggtatctttatttccacggcagcttcgtggatacgtctcattgattctcatggcatcactttccccatgtaggtgggcagacattgttacccctgtttaataaacaaggaaccaacagaggcttaggagaggagttgcctgatgtcgcatgattggtggcagagccaggatcaacagtggggcagggtggggggacctggccaggcagagactggatgagacctggggtgaggaatggcaggcacccagtcagggcagaaaacgagggttgggacttactttgagttttggattggatcagtaaattcccaagaaagagggagactaggaggctagtgaagaactctggagtaaaggggaggattactaagggacatggagtacctatcatgtgtcggacgcttatctatatctctcccatctgaacaaatccttacaggaaccccaggagacaggttatctccactctgcaaattggaaaacagatccagacagtttcagttatgtgtctgagaagttcatttatgtgtccaagacacattcttagctaaaaagctaagcattctgaattggaacccagagaatttgactcccagactctggatcttttcactgctgtgatccatctgggaaaggctagtgatgtgggcaaggggcttattgccccttggtgtttggttgggagtggtcggattggtgggttgggggcacaaggcagccagatctgggactcctgtgcttgtgactggactacaaagagttaaagaacgttgggcctcctcctcccgcctcctgtggcctcctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgagaccagggacaaagggaagagtgggctggtgggcgaggcaccttccggctggcgtgggccctctccgggagggggccgagcctctcctgcccgggcctggtcctggcgccagcctcaggcctgcaggtcctaacctcagccactgccagtgtggg

SEQ ID NO: 44 - DNA, 2500 bp, Homo sapiens
ggaacactgtgccctttccttcccaaaccctcacccctgggcctgaccctggagtcaagaactgcacttggcagagttcagagagaagtcgcctgctctcaagcctgggtcggcctggagaaggggagaagccccatgttgcatagagattaagcttgaattcgttcccaggatgggacctaagcctgggaggatccccaccttcaggaggtgatggtgccattcactgggatagggaaactggcaggaggagccggttgttgagggttgggggtgcagacagtctgttggtcccattggtcttgaggtgtgtgggggacaactagagagaactattgtcccctgcttccacgaggtgacatctccctcctgccttcagctctattttagggatcaaggagtccttggagctgctcctaggcattctcataagggccagaagagctctgcatgtctgtccagcggctctgcagagttaaggctgtgggtcataccagccacaccctcagcgtggtccatgtcacagaaacatgtggaaaccaggtgatttcccagcctcctggtcaggaggtggggttttccatccccagggagtcacttcccagtttctcactcagcactcagcacctagaagcaccatccttgcaggctccactgtggggctggtggccgggagcaagggtctctgtacctcttcaggcagcctgcagtgaggaaacagggaacagaatagcctcctgccacccagcccaccccaacccttagaaatgccctatgtgctgggggctaggacccccgtaatccagggctggaggaatgtgctctggccccgcagggctggccctgtattagggctggcactcaaagtggggatggggggacagattgcagctggaatgggtgctcggtcatgatgtaaatcctggggaagccagctctcctgggcctggtctcagcagcccctcagggccctgcagcttccctggtgacattctctcccagcctctgttcatctgccccctgcctgggcaggagacttgagcagggaagtgcagagtcttctccctgtgagaaggctggatgcgtgtttaaggataaatgaacacgcgaagagtagtaacaacagccaagatttataaatgcctattgttatatatgtagatacttacttaagtatatataaagtacagactgcattatgtatattacatatctttaaatttttagaatagtcctatgaggtcagttcagagatccagacccaggtggtctggctctagagtctaaacaggccgagtgcagtggctcacacctataatcccagcaccttgggaggccagaggcgggaagatcacttgagggtgggaagaacacgtgagctcaggagttcgagaccagcctggacaacatggcgaaaccccatctctataaagaaatcagcctagcatggtggcccgagcctgtagtcccagctactcgggaggctgaggtgggaggatcgcttgagcgcaggagttggaggctgcagtgagctatgggtgaaagagtgagaccttgtctcaaaaaaaattaaaaaataagaattaaatatatttaaaatagagtctaaataagtgaatgatctagaattctcttggttcccctaaagcagctgtcagctttgggggatgtttttccaaattagtgcctcaccctcacgggacagggaagcctgtgggagctgggagggcaggtggaggccgggcaggtggaggccgggcaggtggagatggtgattcgaagaggaggggacgataggaggaggttgaggggtcacccgagtgctgggagtgacgctggaggtatcggcccagcgatgctggagtggtcgggtcggaggcccagcagcgtctggggaggggcgtgggggaggcgtgtccaaggccggctggccccgccccgccccgccgcctggcctctggcccgctggggcgcgggctttcgctttcagtcgagggctagcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtgggtgactcgcgcggcccacgggggacagccgccccgcatgtccacccggctggtgcgcagccttcgggtgcccgggccgcgctctcccggggcgctgtcacgggctgggaggctgggagtcccagtcgccgccccccatccgcatcagacacgcgcgcctctggggacccgctggggactccgggcccggcacacgtgcgctcggggcacaactctggcccccgaagcccctcttacccgcgtcgtgacaggggcgcaccctggttcctccgcgagcgcaggccggggcatttggggctgagcagcgggtcctgggcagacccccgctag

SEQ ID NO: 45 - protein, 7 aa, Simian virus 40
PKKKRKV

SEQ ID NO: 46 - protein, 6 aa, Artificial Sequence
  Synthetic construct
GGSGGS

SEQ ID NO: 47 - DNA, 18 bp, Homo sapiens
cttcatgatgcaggccca

SEQ ID NO: 48 - DNA, 18 bp, Homo sapiens
gtgggtcaccaggcagcc

SEQ ID NO: 49 - DNA, 18 bp, Homo sapiens
cgccgcctggtccctcct

SEQ ID NO: 50 - DNA, 18 bp, Homo sapiens
ctagacgtccgggcagcc

SEQ ID NO: 51 - DNA, 18 bp, Homo sapiens
caggtacaacctcaaggc

SEQ ID NO: 52 - DNA, 18 bp, Homo sapiens
cgccctctgggacgtgct

SEQ ID NO: 53 - DNA, 18 bp, Homo sapiens
ggcgccgccgtccaggaa

SEQ ID NO: 54 - DNA, 18 bp, Homo sapiens
cgaggaggcgggggcggc

SEQ ID NO: 55 - DNA, 18 bp, Homo sapiens
ggcgcggaccgtccccca

SEQ ID NO: 56 - DNA, 18 bp, Homo sapiens
ggggcagcccaggctcag

SEQ ID NO: 57 - DNA, 18 bp, Homo sapiens
cgggcccgagcaggaggg

SEQ ID NO: 58 - DNA, 18 bp, Homo sapiens
cacgccgccgtcgcgggt

SEQ ID NO: 59 - DNA, 18 bp, Homo sapiens
ggtgggggaccccttgac

SEQ ID NO: 60 - DNA, 18 bp, Homo sapiens
gctgctcttcatcgaggt

SEQ ID NO: 61 - DNA, 18 bp, Homo sapiens
ggacggggggaggggggc

SEQ ID NO: 62 - DNA, 18 bp, Homo sapiens
ccccgccccgccccaggc

SEQ ID NO: 63 - DNA, 18 bp, Homo sapiens
cccccagcccttccacat

SEQ ID NO: 64 - DNA, 18 bp, Homo sapiens
cttctagcccctcctccc

SEQ ID NO: 65 - DNA, 18 bp, Homo sapiens
cgccccgtccgcgcaccc

SEQ ID NO: 66 - DNA, 18 bp, Homo sapiens
ggccccggcggcgccgcc

SEQ ID NO: 67 - DNA, 18 bp, Homo sapiens
cgccgccgggtacccggg

SEQ ID NO: 68 - DNA, 18 bp, Homo sapiens
ttcggcaccagctcccac

SEQ ID NO: 69 - DNA, 18 bp, Homo sapiens
cgggccgccgcgggccgc

SEQ ID NO: 70 - DNA, 18 bp, Homo sapiens
ccggccgcaggtgacccg

SEQ ID NO: 71 - DNA, 18 bp, Homo sapiens
ccccttccaccacccccc

SEQ ID NO: 72 - DNA, 18 bp, Homo sapiens
ccccgccccccagttgtt

SEQ ID NO: 73 - DNA, 18 bp, Homo sapiens
cgcccccccttctcccag

SEQ ID NO: 74 - DNA, 18 bp, Homo sapiens
gggaagtgggggggcatg

SEQ ID NO: 75 - DNA, 18 bp, Homo sapiens
tcctggatctactccaag

SEQ ID NO: 76 - DNA, 18 bp, Homo sapiens
atcgggagcagggcggcc

SEQ ID NO: 77 - DNA, 18 bp, Homo sapiens
ggcggggccggcgggagc

SEQ ID NO: 78 - DNA, 18 bp, Homo sapiens
cgaggccgccgcggcggc

SEQ ID NO: 79 - DNA, 18 bp, Homo sapiens
agcgtcgaacggccacag

SEQ ID NO: 80 - DNA, 18 bp, Homo sapiens
ggaggggctggtctagga

SEQ ID NO: 81 - DNA, 18 bp, Homo sapiens
ctaggggctggacgtcga

SEQ ID NO: 82 - DNA, 18 bp, Homo sapiens
gcacaggctcgagcagca

SEQ ID NO: 83 - DNA, 18 bp, Homo sapiens
cgggaggcgggaggagga

SEQ ID NO: 84 - DNA, 18 bp, Homo sapiens
agccagatctgggactcc

SEQ ID NO: 85 - DNA, 18 bp, Homo sapiens
acgttgggcctcctcctc

SEQ ID NO: 86 - DNA, 18 bp, Homo sapiens
gtgtccaaggccggctgg

SEQ ID NO: 87 - DNA, 18 bp, Homo sapiens
gcccgctggggcgcgggc

SEQ ID NO: 88 - protein, 168 aa, Artificial Sequence
  Synthetic construct
GEKPYKCPECGKSFSTSHSLTEHQRTHTGEKPYKCPECGKSFSDPGHLVRHQRTHTGEKPYKCPECGKSFSQSGDLRRHQRTHTGEKPYKCPECGKSFSTSGNLVRHQRTHTGEKPYKCPECGKSFSTSGNLTEHQRTHTGEKPYKCPECGKSFSTTGALTEHQRTHT

SEQ ID NO: 89 - protein, 168 aa, Artificial Sequence
  Synthetic construct
GEKPYKCPECGKSFSDCRDLARHQRTHTGEKPYKCPECGKSFSQSGDLRRHQRTHTGEKPYKCPECGKSFSRADNLTEHQRTHTGEKPYKCPECGKSFSSKKALTEHQRTHTGEKPYKCPECGKSFSTSGHLVRHQRTHTGEKPYKCPECGKSFSRSDELVRHQRTHT

SEQ ID NO: 90 - protein, 168 aa, Artificial Sequence
  Synthetic construct
GEKPYKCPECGKSFSTKNSLTEHQRTHTGEKPYKCPECGKSFSTKNSLTEHQRTHTGEKPYKCPECGKSFSDPGALVRHQRTHTGEKPYKCPECGKSFSRNDALTEHQRTHTGEKPYKCPECGKSFSHTGHLLEHQRTHTGEKPYKCPECGKSFSHTGHLLEHQRTHT

SEQ ID NO: 91 - protein, 168 aa, Artificial Sequence
  Synthetic construct
GEKPYKCPECGKSFSDCRDLARHQRTHTGEKPYKCPECGKSFSQSGDLRRHQRTHTGEKPYKCPECGKSFSRSDKLTEHQRTHTGEKPYKCPECGKSFSDPGALVRHQRTHTGEKPYKCPECGKSFSDPGNLVRHQRTHTGEKPYKCPECGKSFSQNSTLTEHQRTHT

SEQ ID NO: 92 - protein, 168 aa, Artificial Sequence
  Synthetic construct
GEKPYKCPECGKSFSDPGHLVRHQRTHTGEKPYKCPECGKSFSQSGNLTEHQRTHTGEKPYKCPECGKSFSTKNSLTEHQRTHTGEKPYKCPECGKSFSQSGNLTEHQRTHTGEKPYKCPECGKSFSQSSSLVRHQRTHTGEKPYKCPECGKSFSRADNLTEHQRTHT

SEQ ID NO: 93 - protein, 168 aa, Artificial Sequence
  Synthetic construct
GEKPYKCPECGKSFSTSGELVRHQRTHTGEKPYKCPECGKSFSSRRTCRAHQRTHTGEKPYKCPECGKSFSQRAHLERHQRTHTGEKPYKCPECGKSFSRNDALTEHQRTHTGEKPYKCPECGKSFSTKNSLTEHQRTHTGEKPYKCPECGKSFSHTGHLLEHQRTHT

SEQ ID NO: 94 - protein, 168 aa, Artificial Sequence
  Synthetic construct
GEKPYKCPECGKSFSQSSNLVRHQRTHTGEKPYKCPECGKSFSRADNLTEHQRTHTGEKPYKCPECGKSFSDPGALVRHQRTHTGEKPYKCPECGKSFSDCRDLARHQRTHTGEKPYKCPECGKSFSDCRDLARHQRTHTGEKPYKCPECGKSFSDPGHLVRHQRTHT

SEQ ID NO: 95 - protein, 168 aa, Artificial Sequence
  Synthetic construct
GEKPYKCPECGKSFSDPGHLVRHQRTHTGEKPYKCPECGKSFSDPGHLVRHQRTHTGEKPYKCPECGKSFSRSDKLVRHQRTHTGEKPYKCPECGKSFSDPGHLVRHQRTHTGEKPYKCPECGKSFSQRAHLERHQRTHTGEKPYKCPECGKSFSQSGHLTEHQRTHT

SEQ ID NO: 96 - protein, 168 aa, Artificial Sequence
  Synthetic construct
GEKPYKCPECGKSFSTSHSLTEHQRTHTGEKPYKCPECGKSFSSKKHLAEHQRTHTGEKPYKCPECGKSFSSRRTCRAHQRTHTGEKPYKCPECGKSFSDPGNLVRHQRTHTGEKPYKCPECGKSFSRSDDLVRHQRTHTGEKPYKCPECGKSFSDPGHLVRHQRTHT

SEQ ID NO: 97 - protein, 168 aa, Artificial Sequence
  Synthetic construct
GEKPYKCPECGKSFSRADNLTEHQRTHTGEKPYKCPECGKSFSTSGELVRHQRTHTGEKPYKCPECGKSFSRADNLTEHQRTHTGEKPYKCPECGKSFSDCRDLARHQRTHTGEKPYKCPECGKSFSQSGDLRRHQRTHTGEKPYKCPECGKSFSRSDKLVRHQRTHT

SEQ ID NO: 98 - protein, 168 aa, Artificial Sequence
  Synthetic construct
GEKPYKCPECGKSFSRSDKLVRHQRTHTGEKPYKCPECGKSFSQRAHLERHQRTHTGEKPYKCPECGKSFSQSGDLRRHQRTHTGEKPYKCPECGKSFSQSGHLTEHQRTHTGEKPYKCPECGKSFSDCRDLARHQRTHTGEKPYKCPECGKSFSRSDKLTEHQRTHT

SEQ ID NO: 99 - protein, 168 aa, Artificial Sequence
  Synthetic construct
GEKPYKCPECGKSFSTSGHLVRHQRTHTGEKPYKCPECGKSFSRSDDLVRHQRTHTGEKPYKCPECGKSFSDPGALVRHQRTHTGEKPYKCPECGKSFSDCRDLARHQRTHTGEKPYKCPECGKSFSDCRDLARHQRTHTGEKPYKCPECGKSFSSKKALTEHQRTHT

SEQ ID NO: 100 - protein, 168 aa, Artificial Sequence
  Synthetic construct
GEKPYKCPECGKSFSDPGNLVRHQRTHTGEKPYKCPECGKSFSTTGALTEHQRTHTGEKPYKCPECGKSFSSKKHLAEHQRTHTGEKPYKCPECGKSFSQRAHLERHQRTHTGEKPYKCPECGKSFSRSDKLVRHQRTHTGEKPYKCPECGKSFSTSGHLVRHQRTHT

SEQ ID NO: 101 - protein, 168 aa, Artificial Sequence
  Synthetic construct
GEKPYKCPECGKSFSTSGHLVRHQRTHTGEKPYKCPECGKSFSQSGHLTEHQRTHTGEKPYKCPECGKSFSTSGNLTEHQRTHTGEKPYKCPECGKSFSTTGALTEHQRTHTGEKPYKCPECGKSFSTSGELVRHQRTHTGEKPYKCPECGKSFSTSGELVRHQRTHT

SEQ ID NO: 102 - protein, 168 aa, Artificial Sequence
  Synthetic construct
GEKPYKCPECGKSFSDPGHLVRHQRTHTGEKPYKCPECGKSFSRSDKLVRHQRTHTGEKPYKCPECGKSFSRSDNLVRHQRTHTGEKPYKCPECGKSFSRSDKLVRHQRTHTGEKPYKCPECGKSFSRSDKLTEHQRTHTGEKPYKCPECGKSFSQRAHLERHQRTHT

SEQ ID NO: 103 - protein, 168 aa, Artificial Sequence
  Synthetic construct
GEKPYKCPECGKSFSDPGHLVRHQRTHTGEKPYKCPECGKSFSTSHSLTEHQRTHTGEKPYKCPECGKSFSDCRDLARHQRTHTGEKPYKCPECGKSFSSKKHLAEHQRTHTGEKPYKCPECGKSFSHTGHLLEHQRTHTGEKPYKCPECGKSFSSKKHLAEHQRTHT

SEQ ID NO: 104 - protein, 168 aa, Artificial Sequence
  Synthetic construct
GEKPYKCPECGKSFSTSGNLTEHQRTHTGEKPYKCPECGKSFSTSHSLTEHQRTHTGEKPYKCPECGKSFSTTGALTEHQRTHTGEKPYKCPECGKSFSDCRDLARHQRTHTGEKPYKCPECGKSFSTSHSLTEHQRTHTGEKPYKCPECGKSFSSKKHLAEHQRTHT

SEQ ID NO: 105 - protein, 168 aa, Artificial Sequence
  Synthetic construct
GEKPYKCPECGKSFSSKKHLAEHQRTHTGEKPYKCPECGKSFSTKNSLTEHQRTHTGEKPYKCPECGKSFSTKNSLTEHQRTHTGEKPYKCPECGKSFSDCRDLARHQRTHTGEKPYKCPECGKSFSQNSTLTEHQRTHTGEKPYKCPECGKSFSTTGALTEHQRTHT

SEQ ID NO: 106 - protein, 168 aa, Artificial Sequence
  Synthetic construct
GEKPYKCPECGKSFSSKKHLAEHQRTHTGEKPYKCPECGKSFSQSGDLRRHQRTHTGEKPYKCPECGKSFSHTGHLLEHQRTHTGEKPYKCPECGKSFSDPGALVRHQRTHTGEKPYKCPECGKSFSSKKHLAEHQRTHTGEKPYKCPECGKSFSHTGHLLEHQRTHT

SEQ ID NO: 107 - protein, 168 aa, Artificial Sequence
  Synthetic construct
GEKPYKCPECGKSFSDCRDLARHQRTHTGEKPYKCPECGKSFSDCRDLARHQRTHTGEKPYKCPECGKSFSDPGHLVRHQRTHTGEKPYKCPECGKSFSDPGHLVRHQRTHTGEKPYKCPECGKSFSSKKHLAEHQRTHTGEKPYKCPECGKSFSDPGHLVRHQRTHT

SEQ ID NO: 108 - protein, 168 aa, Artificial Sequence
  Synthetic construct
GEKPYKCPECGKSFSRSDKLVRHQRTHTGEKPYKCPECGKSFSSKKHLAEHQRTHTGEKPYKCPECGKSFSQSSSLVRHQRTHTGEKPYKCPECGKSFSRSDKLTEHQRTHTGEKPYKCPECGKSFSHTGHLLEHQRTHTGEKPYKCPECGKSFSHTGHLLEHQRTHT

SEQ ID NO: 109 - protein, 168 aa, Artificial Sequence
  Synthetic construct
GEKPYKCPECGKSFSQSSNLVRHQRTHTGEKPYKCPECGKSFSDCRDLARHQRTHTGEKPYKCPECGKSFSTSGHLVRHQRTHTGEKPYKCPECGKSFSTSGELVRHQRTHTGEKPYKCPECGKSFSQRAHLERHQRTHTGEKPYKCPECGKSFSRSDELVRHQRTHT

SEQ ID NO: 110 - protein, 168 aa, Artificial Sequence
  Synthetic construct
GEKPYKCPECGKSFSHTGHLLEHQRTHTGEKPYKCPECGKSFSDPGHLVRHQRTHTGEKPYKCPECGKSFSRSDDLVRHQRTHTGEKPYKCPECGKSFSDCRDLARHQRTHTGEKPYKCPECGKSFSDCRDLARHQRTHTGEKPYKCPECGKSFSRSDKLTEHQRTHT

SEQ ID NO: 111 - protein, 168 aa, Artificial Sequence
  Synthetic construct
GEKPYKCPECGKSFSRNDTLTEHQRTHTGEKPYKCPECGKSFSDPGNLVRHQRTHTGEKPYKCPECGKSFSTSGHLVRHQRTHTGEKPYKCPECGKSFSQSGDLRRHQRTHTGEKPYKCPECGKSFSDCRDLARHQRTHTGEKPYKCPECGKSFSRNDTLTEHQRTHT

SEQ ID NO: 112 - protein, 168 aa, Artificial Sequence
  Synthetic construct
GEKPYKCPECGKSFSSKKHLAEHQRTHTGEKPYKCPECGKSFSSKKHLAEHQRTHTGEKPYKCPECGKSFSTSHSLTEHQRTHTGEKPYKCPECGKSFSTSHSLTEHQRTHTGEKPYKCPECGKSFSTTGALTEHQRTHTGEKPYKCPECGKSFSSKKHLAEHQRTHT

SEQ ID NO: 113 - protein, 168 aa, Artificial Sequence
  Synthetic construct
GEKPYKCPECGKSFSTSGSLVRHQRTHTGEKPYKCPECGKSFSTSGSLVRHQRTHTGEKPYKCPECGKSFSTSHSLTEHQRTHTGEKPYKCPECGKSFSSKKHLAEHQRTHTGEKPYKCPECGKSFSHTGHLLEHQRTHTGEKPYKCPECGKSFSSKKHLAEHQRTHT

SEQ ID NO: 114 - protein, 168 aa, Artificial Sequence
  Synthetic construct
GEKPYKCPECGKSFSRSDDLVRHQRTHTGEKPYKCPECGKSFSRSDKLVRHQRTHTGEKPYKCPECGKSFSRSDKLVRHQRTHTGEKPYKCPECGKSFSQSSNLVRHQRTHTGEKPYKCPECGKSFSQRAHLERHQRTHTGEKPYKCPECGKSFSRNDALTEHQRTHT

SEQ ID NO: 115 - protein, 168 aa, Artificial Sequence
  Synthetic construct
GEKPYKCPECGKSFSSKKHLAEHQRTHTGEKPYKCPECGKSFSTTGALTEHQRTHTGEKPYKCPECGKSFSTSHSLTEHQRTHTGEKPYKCPECGKSFSSKKHLAEHQRTHTGEKPYKCPECGKSFSDCRDLARHQRTHTGEKPYKCPECGKSFSTSGNLTEHQRTHT

SEQ ID NO: 116 - protein, 168 aa, Artificial Sequence
  Synthetic construct
GEKPYKCPECGKSFSQRAHLERHQRTHTGEKPYKCPECGKSFSTSHSLTEHQRTHTGEKPYKCPECGKSFSTSGNLVRHQRTHTGEKPYKCPECGKSFSQSSSLVRHQRTHTGEKPYKCPECGKSFSQRAHLERHQRTHTGEKPYKCPECGKSFSTTGALTEHQRTHT

SEQ ID NO: 117 - protein, 168 aa, Artificial Sequence
  Synthetic construct
GEKPYKCPECGKSFSTSGNLVRHQRTHTGEKPYKCPECGKSFSSKKHLAEHQRTHTGEKPYKCPECGKSFSTSGELVRHQRTHTGEKPYKCPECGKSFSTKNSLTEHQRTHTGEKPYKCPECGKSFSHTGHLLEHQRTHTGEKPYKCPECGKSFSDPGHLVRHQRTHT

SEQ ID NO: 118 - protein, 168 aa, Artificial Sequence
  Synthetic construct
GEKPYKCPECGKSFSDCRDLARHQRTHTGEKPYKCPECGKSFSSKKHLAEHQRTHTGEKPYKCPECGKSFSDPGHLVRHQRTHTGEKPYKCPECGKSFSDCRDLARHQRTHTGEKPYKCPECGKSFSSKKHLAEHQRTHTGEKPYKCPECGKSFSTSGELVRHQRTHT

SEQ ID NO: 119 - protein, 168 aa, Artificial Sequence
  Synthetic construct
GEKPYKCPECGKSFSDPGHLVRHQRTHTGEKPYKCPECGKSFSDPGHLVRHQRTHTGEKPYKCPECGKSFSHTGHLLEHQRTHTGEKPYKCPECGKSFSHTGHLLEHQRTHTGEKPYKCPECGKSFSDPGHLVRHQRTHTGEKPYKCPECGKSFSQSGHLTEHQRTHT

SEQ ID NO: 120 - protein, 168 aa, Artificial Sequence
  synthetic construct
GEKPYKCPECGKSFSTKNSLTEHQRTHTGEKPYKCPECGKSFSQSGHLTEHQRTHTGEKPYKCPECGKSFSQSGHLTEHQRTHTGEKPYKCPECGKSFSTSGSLVRHQRTHTGEKPYKCPECGKSFSTSGNLVRHQRTHTGEKPYKCPECGKSFSRSDHLTNHQRTHT

SEQ ID NO: 121 - protein, 168 aa, Artificial Sequence
  Synthetic construct
GEKPYKCPECGKSFSQRAHLERHQRTHTGEKPYKCPECGKSFSQNSTLTEHQRTHTGEKPYKCPECGKSFSTSGHLVRHQRTHTGEKPYKCPECGKSFSTSGELVRHQRTHTGEKPYKCPECGKSFSRSDKLVRHQRTHTGEKPYKCPECGKSFSQRAHLERHQRTHT

SEQ ID NO: 122 - protein, 168 aa, Artificial Sequence
  Synthetic construct
GEKPYKCPECGKSFSQSGHLTEHQRTHTGEKPYKCPECGKSFSSRRTCRAHQRTHTGEKPYKCPECGKSFSQRAHLERHQRTHTGEKPYKCPECGKSFSTSGELVRHQRTHTGEKPYKCPECGKSFSRSDKLVRHQRTHTGEKPYKCPECGKSFSQNSTLTEHQRTHT

SEQ ID NO: 123 - protein, 168 aa, Artificial Sequence
  Synthetic construct
GEKPYKCPECGKSFSQSGDLRRHQRTHTGEKPYKCPECGKSFSQSGDLRRHQRTHTGEKPYKCPECGKSFSQSGHLTEHQRTHTGEKPYKCPECGKSFSTSGELVRHQRTHTGEKPYKCPECGKSFSRADNLTEHQRTHTGEKPYKCPECGKSFSQSGDLRRHQRTHT

SEQ ID NO: 124 - protein, 168 aa, Artificial Sequence
  Synthetic construct
GEKPYKCPECGKSFSQRAHLERHQRTHTGEKPYKCPECGKSFSQRAHLERHQRTHTGEKPYKCPECGKSFSQRAHLERHQRTHTGEKPYKCPECGKSFSRSDDLVRHQRTHTGEKPYKCPECGKSFSRSDNLVRHQRTHTGEKPYKCPECGKSFSRSDKLTEHQRTHT

SEQ ID NO: 125 - protein, 168 aa, Artificial Sequence
  Synthetic construct
GEKPYKCPECGKSFSTSGELVRHQRTHTGEKPYKCPECGKSFSRNDALTEHQRTHTGEKPYKCPECGKSFSTSGNLVRHQRTHTGEKPYKCPECGKSFSTSHSLTEHQRTHTGEKPYKCPECGKSFSDPGALVRHQRTHTGEKPYKCPECGKSFSQRAHLERHQRTHT

SEQ ID NO: 126 - protein, 168 aa, Artificial Sequence
  Synthetic construct
GEKPYKCPECGKSFSSRRTCRAHQRTHTGEKPYKCPECGKSFSQSGNLTEHQRTHTGEKPYKCPECGKSFSDCRDLARHQRTHTGEKPYKCPECGKSFSRSDNLVRHQRTHTGEKPYKCPECGKSFSRSDNLVRHQRTHTGEKPYKCPECGKSFSRSDNLVRHQRTHT

SEQ ID NO: 127 - protein, 168 aa, Artificial Sequence
  Synthetic construct
GEKPYKCPECGKSFSSKKALTEHQRTHTGEKPYKCPECGKSFSQRAHLERHQRTHTGEKPYKCPECGKSFSTTGALTEHQRTHTGEKPYKCPECGKSFSDPGHLVRHQRTHTGEKPYKCPECGKSFSDCRDLARHQRTHTGEKPYKCPECGKSFSTSHSLTEHQRTHT

SEQ ID NO: 128 - protein, 168 aa, Artificial Sequence
  Synthetic construct
GEKPYKCPECGKSFSDPGHLVRHQRTHTGEKPYKCPECGKSFSRSDDLVRHQRTHTGEKPYKCPECGKSFSTSHSLTEHQRTHTGEKPYKCPECGKSFSDCRDLARHQRTHTGEKPYKCPECGKSFSHTGHLLEHQRTHTGEKPYKCPECGKSFSDCRDLARHQRTHT

SEQ ID NO: 129 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSHSLTEHQRTHTGEKPYKCPECGKSFSDPGHLVRHQRTHTGEKPYKCPECGKSFSQSGDLRRHQRTHTGEKPYKCPECGKSFSTSGNLVRHQRTHTGEKPYKCPECGKSFSTSGNLTEHQRTHTGEKPYKCPECGKSFSTTGALTEHQRTHTGEQKLISEEDLEQKLISEEDLEQKLISEEDL

SEQ ID NO: 130 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDCRDLARHQRTHTGEKPYKCPECGKSFSQSGDLRRHQRTHTGEKPYKCPECGKSFSRADNLTEHQRTHTGEKPYKCPECGKSFSSKKALTEHQRTHTGEKPYKCPECGKSFSTSGHLVRHQRTHTGEKPYKCPECGKSFSRSDELVRHQRTHTGEQKLISEEDLEQKLISEEDLEQKLISEEDL

SEQ ID NO: 131 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTKNSLTEHQRTHTGEKPYKCPECGKSFSTKNSLTEHQRTHTGEKPYKCPECGKSFSDPGALVRHQRTHTGEKPYKCPECGKSFSRNDALTEHQRTHTGEKPYKCPECGKSFSHTGHLLEHQRTHTGEKPYKCPECGKSFSHTGHLLEHQRTHTGEQKLISEEDLEQKLISEEDLEQKLISEEDL

SEQ ID NO: 132 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDCRDLARHQRTHTGEKPYKCPECGKSFSQSGDLRRHQRTHTGEKPYKCPECGKSFSRSDKLTEHQRTHTGEKPYKCPECGKSFSDPGALVRHQRTHTGEKPYKCPECGKSFSDPGNLVRHQRTHTGEKPYKCPECGKSFSQNSTLTEHQRTHTGEQKLISEEDLEQKLISEEDLEQKLISEEDL

SEQ ID NO: 133 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDPGHLVRHQRTHTGEKPYKCPECGKSFSQSGNLTEHQRTHTGEKPYKCPECGKSFSTKNSLTEHQRTHTGEKPYKCPECGKSFSQSGNLTEHQRTHTGEKPYKCPECGKSFSQSSSLVRHQRTHTGEKPYKCPECGKSFSRADNLTEHQRTHTGEQKLISEEDLEQKLISEEDLEQKLISEEDL

SEQ ID NO: 134 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGELVRHQRTHTGEKPYKCPECGKSFSSRRTCRAHQRTHTGEKPYKCPECGKSFSQRAHLERHQRTHTGEKPYKCPECGKSFSRNDALTEHQRTHTGEKPYKCPECGKSFSTKNSLTEHQRTHTGEKPYKCPECGKSFSHTGHLLEHQRTHTGEQKLISEEDLEQKLISEEDLEQKLISEEDL

SEQ ID NO: 135 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQSSNLVRHQRTHTGEKPYKCPECGKSFSRADNLTEHQRTHTGEKPYKCPECGKSFSDPGALVRHQRTHTGEKPYKCPECGKSFSDCRDLARHQRTHTGEKPYKCPECGKSFSDCRDLARHQRTHTGEKPYKCPECGKSFSDPGHLVRHQRTHTGEQKLISEEDLEQKLISEEDLEQKLISEEDL

SEQ ID NO: 136 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDPGHLVRHQRTHTGEKPYKCPECGKSFSDPGHLVRHQRTHTGEKPYKCPECGKSFSRSDKLVRHQRTHTGEKPYKCPECGKSFSDPGHLVRHQRTHTGEKPYKCPECGKSFSQRAHLERHQRTHTGEKPYKCPECGKSFSQSGHLTEHQRTHTGEQKLISEEDLEQKLISEEDLEQKLISEEDL

SEQ ID NO: 137 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSHSLTEHQRTHTGEKPYKCPECGKSFSSKKHLAEHQRTHTGEKPYKCPECGKSFSSRRTCRAHQRTHTGEKPYKCPECGKSFSDPGNLVRHQRTHTGEKPYKCPECGKSFSRSDDLVRHQRTHTGEKPYKCPECGKSFSDPGHLVRHQRTHTGEQKLISEEDLEQKLISEEDLEQKLISEEDL

SEQ ID NO: 138 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSRADNLTEHQRTHTGEKPYKCPECGKSFSTSGELVRHQRTHTGEKPYKCPECGKSFSRADNLTEHQRTHTGEKPYKCPECGKSFSDCRDLARHQRTHTGEKPYKCPECGKSFSQSGDLRRHQRTHTGEKPYKCPECGKSFSRSDKLVRHQRTHTGEQKLISEEDLEQKLISEEDLEQKLISEEDL

SEQ ID NO: 139 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSRSDKLVRHQRTHTGEKPYKCPECGKSFSQRAHLERHQRTHTGEKPYKCPECGKSFSQSGDLRRHQRTHTGEKPYKCPECGKSFSQSGHLTEHQRTHTGEKPYKCPECGKSFSDCRDLARHQRTHTGEKPYKCPECGKSFSRSDKLTEHQRTHTGEQKLISEEDLEQKLISEEDLEQKLISEEDL

SEQ ID NO: 140 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGHLVRHQRTHTGEKPYKCPECGKSFSRSDDLVRHQRTHTGEKPYKCPECGKSFSDPGALVRHQRTHTGEKPYKCPECGKSFSDCRDLARHQRTHTGEKPYKCPECGKSFSDCRDLARHQRTHTGEKPYKCPECGKSFSSKKALTEHQRTHTGEQKLISEEDLEQKLISEEDLEQKLISEEDL

SEQ ID NO: 141 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDPGNLVRHQRTHTGEKPYKCPECGKSFSTTGALTEHQRTHTGEKPYKCPECGKSFSSKKHLAEHQRTHTGEKPYKCPECGKSFSQRAHLERHQRTHTGEKPYKCPECGKSFSRSDKLVRHQRTHTGEKPYKCPECGKSFSTSGHLVRHQRTHTGEQKLISEEDLEQKLISEEDLEQKLISEEDL

SEQ ID NO: 142 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGHLVRHQRTHTGEKPYKCPECGKSFSQSGHLTEHQRTHTGEKPYKCPECGKSFSTSGNLTEHQRTHTGEKPYKCPECGKSFSTTGALTEHQRTHTGEKPYKCPECGKSFSTSGELVRHQRTHTGEKPYKCPECGKSFSTSGELVRHQRTHTGEQKLISEEDLEQKLISEEDLEQKLISEEDL

SEQ ID NO: 143 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDPGHLVRHQRTHTGEKPYKCPECGKSFSRSDKLVRHQRTHTGEKPYKCPECGKSFSRSDNLVRHQRTHTGEKPYKCPECGKSFSRSDKLVRHQRTHTGEKPYKCPECGKSFSRSDKLTEHQRTHTGEKPYKCPECGKSFSQRAHLERHQRTHTGEQKLISEEDLEQKLISEEDLEQKLISEEDL

SEQ ID NO: 144 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDPGHLVRHQRTHTGEKPYKCPECGKSFSTSHSLTEHQRTHTGEKPYKCPECGKSFSDCRDLARHQRTHTGEKPYKCPECGKSFSSKKHLAEHQRTHTGEKPYKCPECGKSFSHTGHLLEHQRTHTGEKPYKCPECGKSFSSKKHLAEHQRTHTGEQKLISEEDLEQKLISEEDLEQKLISEEDL

SEQ ID NO: 145 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGNLTEHQRTHTGEKPYKCPECGKSFSTSHSLTEHQRTHTGEKPYKCPECGKSFSTTGALTEHQRTHTGEKPYKCPECGKSFSDCRDLARHQRTHTGEKPYKCPECGKSFSTSHSLTEHQRTHTGEKPYKCPECGKSFSSKKHLAEHQRTHTGEQKLISEEDLEQKLISEEDLEQKLISEEDL

SEQ ID NO: 146 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSSKKHLAEHQRTHTGEKPYKCPECGKSFSTKNSLTEHQRTHTGEKPYKCPECGKSFSTKNSLTEHQRTHTGEKPYKCPECGKSFSDCRDLARHQRTHTGEKPYKCPECGKSFSQNSTLTEHQRTHTGEKPYKCPECGKSFSTTGALTEHQRTHTGEQKLISEEDLEQKLISEEDLEQKLISEEDL

SEQ ID NO: 147 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSSKKHLAEHQRTHTGEKPYKCPECGKSFSQSGDLRRHQRTHTGEKPYKCPECGKSFSHTGHLLEHQRTHTGEKPYKCPECGKSFSDPGALVRHQRTHTGEKPYKCPECGKSFSSKKHLAEHQRTHTGEKPYKCPECGKSFSHTGHLLEHQRTHTGEQKLISEEDLEQKLISEEDLEQKLISEEDL

SEQ ID NO: 148 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDCRDLARHQRTHTGEKPYKCPECGKSFSDCRDLARHQRTHTGEKPYKCPECGKSFSDPGHLVRHQRTHTGEKPYKCPECGKSFSDPGHLVRHQRTHTGEKPYKCPECGKSFSSKKHLAEHQRTHTGEKPYKCPECGKSFSDPGHLVRHQRTHTGEQKLISEEDLEQKLISEEDLEQKLISEEDL

SEQ ID NO: 149 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSRSDKLVRHQRTHTGEKPYKCPECGKSFSSKKHLAEHQRTHTGEKPYKCPECGKSFSQSSSLVRHQRTHTGEKPYKCPECGKSFSRSDKLTEHQRTHTGEKPYKCPECGKSFSHTGHLLEHQRTHTGEKPYKCPECGKSFSHTGHLLEHQRTHTGEQKLISEEDLEQKLISEEDLEQKLISEEDL

SEQ ID NO: 150 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQSSNLVRHQRTHTGEKPYKCPECGKSFSDCRDLARHQRTHTGEKPYKCPECGKSFSTSGHLVRHQRTHTGEKPYKCPECGKSFSTSGELVRHQRTHTGEKPYKCPECGKSFSQRAHLERHQRTHTGEKPYKCPECGKSFSRSDELVRHQRTHTGEQKLISEEDLEQKLISEEDLEQKLISEEDL

SEQ ID NO: 151 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSHTGHLLEHQRTHTGEKPYKCPECGKSFSDPGHLVRHQRTHTGEKPYKCPECGKSFSRSDDLVRHQRTHTGEKPYKCPECGKSFSDCRDLARHQRTHTGEKPYKCPECGKSFSDCRDLARHQRTHTGEKPYKCPECGKSFSRSDKLTEHQRTHTGEQKLISEEDLEQKLISEEDLEQKLISEEDL

SEQ ID NO: 152 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSRNDTLTEHQRTHTGEKPYKCPECGKSFSDPGNLVRHQRTHTGEKPYKCPECGKSFSTSGHLVRHQRTHTGEKPYKCPECGKSFSQSGDLRRHQRTHTGEKPYKCPECGKSFSDCRDLARHQRTHTGEKPYKCPECGKSFSRNDTLTEHQRTHTGEQKLISEEDLEQKLISEEDLEQKLISEEDL

SEQ ID NO: 153 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSSKKHLAEHQRTHTGEKPYKCPECGKSFSSKKHLAEHQRTHTGEKPYKCPECGKSFSTSHSLTEHQRTHTGEKPYKCPECGKSFSTSHSLTEHQRTHTGEKPYKCPECGKSFSTTGALTEHQRTHTGEKPYKCPECGKSFSSKKHLAEHQRTHTGEQKLISEEDLEQKLISEEDLEQKLISEEDL

SEQ ID NO: 154 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGSLVRHQRTHTGEKPYKCPECGKSFSTSGSLVRHQRTHTGEKPYKCPECGKSFSTSHSLTEHQRTHTGEKPYKCPECGKSFSSKKHLAEHQRTHTGEKPYKCPECGKSFSHTGHLLEHQRTHTGEKPYKCPECGKSFSSKKHLAEHQRTHTGEQKLISEEDLEQKLISEEDLEQKLISEEDL

SEQ ID NO: 155 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSRSDDLVRHQRTHTGEKPYKCPECGKSFSRSDKLVRHQRTHTGEKPYKCPECGKSFSRSDKLVRHQRTHTGEKPYKCPECGKSFSQSSNLVRHQRTHTGEKPYKCPECGKSFSQRAHLERHQRTHTGEKPYKCPECGKSFSRNDALTEHQRTHTGEQKLISEEDLEQKLISEEDLEQKLISEEDL

SEQ ID NO: 156 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSSKKHLAEHQRTHTGEKPYKCPECGKSFSTTGALTEHQRTHTGEKPYKCPECGKSFSTSHSLTEHQRTHTGEKPYKCPECGKSFSSKKHLAEHQRTHTGEKPYKCPECGKSFSDCRDLARHQRTHTGEKPYKCPECGKSFSTSGNLTEHQRTHTGEQKLISEEDLEQKLISEEDLEQKLISEEDL

SEQ ID NO: 157 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDCRDLARHQRTHTGEKPYKCPECGKSFSSKKHLAEHQRTHTGEKPYKCPECGKSFSDPGHLVRHQRTHTGEKPYKCPECGKSFSDCRDLARHQRTHTGEKPYKCPECGKSFSSKKHLAEHQRTHTGEKPYKCPECGKSFSTSGELVRHQRTHTGEQKLISEEDLEQKLISEEDLEQKLISEEDL

SEQ ID NO: 158 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDPGHLVRHQRTHTGEKPYKCPECGKSFSDPGHLVRHQRTHTGEKPYKCPECGKSFSHTGHLLEHQRTHTGEKPYKCPECGKSFSHTGHLLEHQRTHTGEKPYKCPECGKSFSDPGHLVRHQRTHTGEKPYKCPECGKSFSQSGHLTEHQRTHTGEQKLISEEDLEQKLISEEDLEQKLISEEDL

SEQ ID NO: 159 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTKNSLTEHQRTHTGEKPYKCPECGKSFSQSGHLTEHQRTHTGEKPYKCPECGKSFSQSGHLTEHQRTHTGEKPYKCPECGKSFSTSGSLVRHQRTHTGEKPYKCPECGKSFSTSGNLVRHQRTHTGEKPYKCPECGKSFSRSDHLTNHQRTHTGEQKLISEEDLEQKLISEEDLEQKLISEEDL

SEQ ID NO: 160 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQRAHLERHQRTHTGEKPYKCPECGKSFSQNSTLTEHQRTHTGEKPYKCPECGKSFSTSGHLVRHQRTHTGEKPYKCPECGKSFSTSGELVRHQRTHTGEKPYKCPECGKSFSRSDKLVRHQRTHTGEKPYKCPECGKSFSQRAHLERHQRTHTGEQKLISEEDLEQKLISEEDLEQKLISEEDL

SEQ ID NO: 161 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQSGHLTEHQRTHTGEKPYKCPECGKSFSSRRTCRAHQRTHTGEKPYKCPECGKSFSQRAHLERHQRTHTGEKPYKCPECGKSFSTSGELVRHQRTHTGEKPYKCPECGKSFSRSDKLVRHQRTHTGEKPYKCPECGKSFSQNSTLTEHQRTHTGEQKLISEEDLEQKLISEEDLEQKLISEEDL

SEQ ID NO: 162 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQSGDLRRHQRTHTGEKPYKCPECGKSFSQSGDLRRHQRTHTGEKPYKCPECGKSFSQSGHLTEHQRTHTGEKPYKCPECGKSFSTSGELVRHQRTHTGEKPYKCPECGKSFSRADNLTEHQRTHTGEKPYKCPECGKSFSQSGDLRRHQRTHTGEQKLISEEDLEQKLISEEDLEQKLISEEDL

SEQ ID NO: 163 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSQRAHLERHQRTHTGEKPYKCPECGKSFSQRAHLERHQRTHTGEKPYKCPECGKSFSQRAHLERHQRTHTGEKPYKCPECGKSFSRSDDLVRHQRTHTGEKPYKCPECGKSFSRSDNLVRHQRTHTGEKPYKCPECGKSFSRSDKLTEHQRTHTGEQKLISEEDLEQKLISEEDLEQKLISEEDL

SEQ ID NO: 164 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSTSGELVRHQRTHTGEKPYKCPECGKSFSRNDALTEHQRTHTGEKPYKCPECGKSFSTSGNLVRHQRTHTGEKPYKCPECGKSFSTSHSLTEHQRTHTGEKPYKCPECGKSFSDPGALVRHQRTHTGEKPYKCPECGKSFSQRAHLERHQRTHTGEQKLISEEDLEQKLISEEDLEQKLISEEDL

SEQ ID NO: 165 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSSRRTCRAHQRTHTGEKPYKCPECGKSFSQSGNLTEHQRTHTGEKPYKCPECGKSFSDCRDLARHQRTHTGEKPYKCPECGKSFSRSDNLVRHQRTHTGEKPYKCPECGKSFSRSDNLVRHQRTHTGEKPYKCPECGKSFSRSDNLVRHQRTHTGEQKLISEEDLEQKLISEEDLEQKLISEEDL

SEQ ID NO: 166 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSSKKALTEHQRTHTGEKPYKCPECGKSFSQRAHLERHQRTHTGEKPYKCPECGKSFSTTGALTEHQRTHTGEKPYKCPECGKSFSDPGHLVRHQRTHTGEKPYKCPECGKSFSDCRDLARHQRTHTGEKPYKCPECGKSFSTSHSLTEHQRTHTGEQKLISEEDLEQKLISEEDLEQKLISEEDL

SEQ ID NO: 167 - protein, 279 aa, Artificial Sequence
  synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSDPGHLVRHQRTHTGEKPYKCPECGKSFSRSDDLVRHQRTHTGEKPYKCPECGKSFSTSHSLTEHQRTHTGEKPYKCPECGKSFSDCRDLARHQRTHTGEKPYKCPECGKSFSHTGHLLEHQRTHTGEKPYKCPECGKSFSDCRDLARHQRTHTGEQKLISEEDLEQKLISEEDLEQKLISEEDL

SEQ ID NO: 168 - protein, 309 aa, Artificial Sequence
  synthetic construct
MHHHHHHGYGRKKRRQRRRGYPYDVPDYAPWDIMPKKKRKVGLEPGEKPYKCPECGKSFSQRAHLERHQRTHTGEKPYKCPECGKSFSTSHSLTEHQRTHTGEKPYKCPECGKSFSTSGNLVRHQRTHTGEKPYKCPECGKSFSQSSSLVRHQRTHTGEKPYKCPECGKSFSQRAHLERHQRTHTGEKPYKCPECGKSFSTTGALTEHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 169 - protein, 309 aa, Artificial Sequence
  synthetic construct
MHHHHHHGYGRKKRRQRRRGYPYDVPDYAPWDIMPKKKRKVGLEPGEKPYKCPECGKSFSTSGNLVRHQRTHTGEKPYKCPECGKSFSSKKHLAEHQRTHTGEKPYKCPECGKSFSTSGELVRHQRTHTGEKPYKCPECGKSFSTKNSLTEHQRTHTGEKPYKCPECGKSFSHTGHLLEHQRTHTGEKPYKCPECGKSFSDPGHLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 170 - DNA, 4513 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccgtatacctcgagcccggggaaaagccatataaatgccccgagtgcggcaaatcattcagccaaagtagcaacttagtaagacaccagcgcacccataccggtaagaaaactagtcttaagctcgagcccggggaaaaaccctataaatgccccgagtgtggtaagtcattctctcaaagcggggatttaagaagacaccagagaacccacaccggtaagaaaactagtggcgcgccctcgagcccggggagaaaccttataaatgcccagaatgcgggaaatcgttcagtcaaagagcacatttagaaagacatcaacggacccacaccggtaagaaaactagtcctaggctcgagcccggggaaaaaccttacaagtgccctgagtgcggcaagagcttctctcaatcaagttcattagtaagacaccagaggactcataccggtaagaaaactagtcctcagcctcgagcccggggagaagccttataagtgccctgagtgtggcaaaagcttcagcgatcctggaaatttagtaagacaccaacgcacccacaccggtaagaaaactagtatgcatctcgagcccggggaaaaaccgtataaatgtcctgagtgcggtaagtctttttccgactgtagagacttagcgagacaccaacgtactcataccggtaaaaagactagttgtacactcgagcccggggaaaaaccgtacaagtgtcctgagtgcgggaagagtttctccgatccgggccacttagtaagacatcagaggacacataccggtaaaaagactagtttcgaactcgagcccggggagaaaccatacaaatgccccgagtgtggaaagtcatttagtgatccaggcgcattagtaagacatcagcggacacataccggtaagaaaactagtgaattcctcgagcccggggagaagccatataaatgtcccgagtgtggcaagtccttttctagatcagataatttagtaagacatcagagaacgcacaccggtaaaaagactagtcaattgctcgagcccggggagaagccatacaagtgtcccgaatgcgggaagtcattctccagaagtgacgatttagtaagacatcagcgcacgcacaccggtaagaaaactagtccatggctcgagcccggggagaagccctacaagtgtccagaatgcggaaagagtttctccagaagtgacaaattagtaagacaccagagaacccataccggtaagaaaactagtcatatgctcgagcccggggagaagccgtacaagtgccctgaatgtggtaagtcattttcgagaagtgatgaattagtaagacaccagcggactcataccggtaaaaagactagtgctagcctcgagcccggggagaagccctataaatgtccagaatgtggaaagtcctttagcacgtcagggaacttagtaagacaccagcgaactcataccggtaagaaaactagtttaattaactcgagcccggggagaaaccatacaagtgtccagagtgcgggaaaagctttagtacaagcggtgagttagtaagacaccaacgaacacacaccggtaaaaagactagtgtttaaacctcgagcccggggaaaagccctacaagtgcccggaatgcggcaagtcttttagcaccagcggacatttagtaagacaccagagaacccacaccggtaaaaagactagtccgcggctcgagcccggggaaaagccctacaagtgtcctgagtgcggaaagtctttctccactagcggttcattagtaagacaccagaggacacacaccggtaaaaagactagtgcatgcgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 171 - DNA, 4442 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgtatacctcgagcccggggagaagccctataaatgccctgaatgcgggaaatctttctcttctaagaaggcactcacagaacaccagcggacacacaccggtaaaaaaactagtcttaagctcgagcccggggaaaagccctacaagtgccccgaatgcgggaagtcttttagtcagagtggaaatcttaccgagcaccagagaacacacaccggtaagaagactagtggcgcgccctcgagcccggggagaagccatacaagtgccctgaatgtggcaagtccttttcaagagccgataacctgacagaacaccaaaggacgcataccggtaagaaaactagtcctaggctcgagcccggggagaagccctataaatgccctgaatgtggcaagagcttcagtactagcgggaatctcactgaacatcagcgaactcataccggtaaaaaaactagtcctcagcctcgagcccggggaaaaaccatacaagtgccctgagtgcggcaagagttttagtacctcacactctcttacagaacatcagcgaacccacaccggtaaaaaaactagtatgcatctcgagcccggggagaaaccatacaaatgtcccgaatgtggcaagagtttcagcagtaaaaagcatctcgctgagcatcagagaactcacaccggtaaaaagactagttgtacactcgagcccggggaaaagccctacaaatgccccgaatgtggtaagtctttttctaggaacgacaccttgacagaacaccagcggacccacaccggtaagaagactagtgaattcctcgagcccggggagaagccttataagtgccccgaatgtggaaagagtttctctactaagaatagcctgaccgagcaccagcgcactcacaccggtaagaaaactagtcaattgctcgagcccggggagaagccctataaatgccctgaatgcgggaaatctttctctcaatcaggccacctcacagaacaccagcggacacacaccggtaaaaaaactagtccatggctcgagcccggggagaaaccctataagtgtcccgaatgcgggaaatcattctctcatacagggcatctgctcgaacatcaaaggacgcacaccggtaaaaagactagtcatatgctcgagcccggggaaaagccttacaaatgccccgaatgtgggaagagtttcagccggtctgataagctgaccgaacaccagagaactcataccggtaaaaaaactagtgctagcctcgagcccggggaaaagccctacaagtgccctgagtgtgggaagtccttttcttcaagacgcacgtgccgcgctcaccagcggacacataccggtaagaaaactagtttaattaactcgagcccggggagaaaccatacaaatgtcccgaatgtggcaagtccttctcacagaactctactttgaccgagcatcagagaactcacaccggtaagaagactagtccgcggctcgagcccggggaaaagccttataagtgccccgaatgcggaaagagcttctcaaggaatgatgcacttaccgagcatcaaaggactcataccggtaaaaaaactagtgcatgcttcgaactcgagcccggggaaaagccctataagtgtcccgaatgcggcaagagttttagtactactggcgcactcacagaacaccagcgcactcacaccggtaagaaaactagtgaaagtcctctccactgactgtagcctccaattcactggagatctgacacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 172 - DNA, 4376 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccgtatacctcgagcccggggagaagccatacaaatgccctgagtgtggaaagtcatttagccagcgagctaatctgcgggcccaccagcggacccacaccggtaagaagactagtcttaagctcgagcccggggagaagccatacaaatgtccagaatgtggaaagtccttctctgatagtggcaacctcagagtgcatcagcgaacacataccggtaagaagactagtggcgcgccctcgagcccggggaaaagccatataagtgccctgagtgtggaaagagcttcagtaggaaggataaccttaaaaaccaccaaagaacccacaccggtaagaagactagtcctaggctcgagcccggggaaaagccatataaatgtcccgagtgcggcaaatccttctctaccactggcaacctcacagtgcatcaacggactcacaccggtaaaaagactagtcctcagcctcgagcccggggaaaagccctataaatgtcccgagtgcggaaagtctttttccagccctgccgacctgacacgccaccaacgaacgcacaccggtaagaagactagtatgcatctcgagcccggggaaaagccgtacaaatgtccagagtgtggaaaatccttttctgataaaaaggacctgacacggcatcagcgaacccacaccggtaaaaagactagttgtacactcgagcccggggagaaaccttataaatgcccagaatgcggtaaaagtttcagcaggacggataccttgcgggatcatcagagaacccacaccggtaaaaaaactagtgaattcctcgagcccggggaaaaaccatacaagtgccccgagtgtggcaagagctttagtacccacctcgacctgattagacaccagcgcacccacaccggtaagaaaactagtcaattgctcgagcccggggaaaagccctataagtgcccagagtgcgggaaatcattctcacagctggcacatcttagagcccaccagcggacccacaccggtaagaagactagtccatggctcgagcccggggagaaaccctataagtgccctgaatgcggcaagtctttcagtgagcggtcacatctccgagagcaccagcgaacgcacaccggtaaaaagactagtcatatgctcgagcccggggaaaaaccctacaagtgccctgagtgtggaaagtcatttagtcgctccgaccacctgaccaaccatcagcggactcacaccggtaagaaaactagtgctagcctcgagcccggggagaaaccttacaagtgccccgagtgcggcaagagtttcagccacaggaccaccctgacaaaccaccagaggacccacaccggtaaaaagactagtttaattaactcgagcccggggagaaaccttataagtgtcctgagtgcggcaaaagtttctctcaaaagtcctcccttattgcccatcaaaggacccataccggtaagaagactagtgtttaaacctcgagcccggggagaagccctataaatgtcccgagtgcggaaagtccttctcacggcgcgatgaattgaacgtccatcagagaacacacaccggtaaaaaaactagtccgcggctcgagcccggggaaaaaccttataagtgtcccgagtgcggcaagagtttcagtcacaaaaacgcacttcagaatcatcagaggacacataccggtaagaaaactagtgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 173 - DNA, 57 bp, Artificial Sequence
  Synthetic construct
tcgacaggcccaggcggccctcgaggatatcatgatgactagtggccaggccggccc

SEQ ID NO: 174 - DNA, 57 bp, Artificial Sequence
  Synthetic construct
aattgggccggcctggccactagtcatcatgatatcctcgagggccgcctgggcctg

SEQ ID NO: 175 - DNA, 6699 bp, Artificial Sequence
  Synthetic construct
gcttgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaagctttgcaaagatggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatctctcgacaggcccaggcggccctcgaggatatcatgatgactagtggccaggccggcccaattccagatctatgaatcgtagatactgaaaaaccccgcaagttcacttcaactgtgcatcgtgcaccatctcaatttctttcatttatacatcgttttgccttcttttatgtaactatactcctctaagtttcaatcttggccatgtaacctctgatctatagaattttttaaatgactagaattaatgcccatcttttttttggacctaaattcttcatgaaaatatattacgagggcttattcagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccggtcgaaattcccctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttggggatctcgactctagctagaggatcaattcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgtt

SEQ ID NO: 176 - DNA, 6481 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcaaagcttggagttgattgtatgcttggtatagcttgaaatattgtgcagaaaaagaaacaaggaagaaagggaacgagaacaatgacgaggaaacaaaagattaataattgcaggtctatttatacttgatagcaaagcggcaaactttttttatttcaaattcaagtaactggaaggaaggccgtataccgttgctcattagagagtagtgtgcgtgaatgaaggaaggaaaaagtttcgtgtgttcgaagatacccctcatcagctctggaacaacgacatctgttggtgctgtctttgtcgttaattttttcctttagtgtcttccatcattttttttgtcattgcggatatggtgagacaacaacgggggagagagaaaagaaaaaaaaagaaaagaagttgcatgcattcatgcgggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 177 - DNA, 6018 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgagggtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 178 - DNA, 23 bp, Artificial Sequence
  Synthetic construct
cgccgcatgcattcatgcaggcc

SEQ ID NO: 179 - DNA, 17 bp, Artificial Sequence
  Synthetic construct
tgcatgaatgcatgcgg

SEQ ID NO: 180 - DNA, 5021 bp, Artificial Sequence
  Synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgcattcatgcaggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 181 - DNA, 6408 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcagcggccgcttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 182 - DNA, 6308 bp, Artificial Sequence
  synthetic construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccgcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaagtccaaagcttctgaataagccctcgtaatatattttcatgaagaatttaggtccaaaaaaaagatgggcattaattctagtcatttaaaaaattctatagatcagaggttacatggccaagattgaaacttagaggagtatagttacataaaagaaggcaaaacgatgtataaatgaaagaaattgagatggtgcacgatgcacagttgaagtgaacttgcggggtttttcagtatctacgattcatagatctggaattgggccggcctggccactagtcatcatgatatcctcgagggccgcctgggcctgtcgagagatctctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcggcggtacccaattcgacctttctcttcttttttggaggctcgggaattaattccgctttatccatctttgcagcggccgcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 183 - DNA, 8068 bp, Artificial Sequence
  synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtttcgaagatatcgttgacattgattattgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcacttaagctggagctttgggaggagacggggaggacagactggaggcgtgggcccactagtgtttagtgaaccgtcagatcgcctggagacgccatccacgctgttttgacctccatagaagacaccgggaccgatccagcctccggactctagcctcgagcccaagcttggtaccgagctcggatccagccaccatgggagtcaaagttctgtttgccctgatctgcatcgctgnggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacaaccggtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattttaattaacgttggcaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggctaggtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 184 - DNA, 6083 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatggatgctaagtccctgacagcgtggagccgcacactggttaccttcaaagatgttttcgtggatttcacccgcgaagagtggaaactgctggataccgcacagcagattgtgtatcgcaacgttatgctggaaaactacaagaatctggttagcctgggctatcagctgacaaaacccgacgtcatcctgcgtctggaaaagggtgaagagccgtggctggttgaacgggagattcaccaggagacacatcctgattctgaaactgcctttgagatcaaaagctccgtcagtccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtgagcagaaactcatctctgaagaagatctggaacaaaagttgatttcagaagaagatctggaacagaagctcatctctgaggaagatctgtaagcggccgcgaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 185 - DNA, 5916 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatgccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtggcggcagcggcggcagcgaattccgcacactggttaccttcaaagatgttttcgtggatttcacccgcgaagagtggaaactgctggataccgcacagcagattgtgtatcgcaacgttatgctggaaaactacaagaatctggttagcctgggctatggatccgagcagaaactcatctctgaagaagatctggaacaaaagttgatttcagaagaagatctggaacagaagctcatctctgaggaagatctgtaagcggccgcaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 186 - DNA, 5897 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggggaaaagccatataaatgccccgagtgcggcaaatcattcagccaaagtagcaacttagtaagacaccagcgcacccataccggtgagcagaaactcatctctgaagaagatctggaacaaaagttgatttcagaagaagatctggaacagaagctcatctctgaggaagatctgtaagcggccgcgaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 187 - DNA, 6198 bp, Artificial Sequence
  Synthetic construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagagatatcatgccgaaaaagaaacgtaaagtggggctcgagcccggggaaaagccctacaagtgccctgagtgtgggaagtccttttcttcaagacgcacgtgccgcgctcaccagcggacacataccggggagaagccctataaatgtccagaatgtggaaagtcctttagcacgtcagggaacttagtaagacaccagcgaactcataccggggagaagccatataaatgtcccgagtgtggcaagtccttttctagatcagataatttagtaagacatcagagaacgcacaccggggaaaagccctacaagtgcccggaatgcggcaagtcttttagcaccagcggacatttagtaagacaccagagaacccacaccggggaaaaaccctataaatgccccgagtgtggtaagtcattctctcaaagcggggatttaagaagacaccagagaacccacaccggggaaaaaccgtataaatgtcctgagtgcggtaagtctttttccgactgtagagacttagcgagacaccaacgtactcataccggtggcggcagcggcggcagcgaattcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaacggatccgagcagaaactcatctctgaagaagatctggaacaaaagttgatttcagaagaagatctggaacagaagctcatctctgaggaagatctgtaagcggccgcaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 188 - DNA, 10723 bp, Artificial Sequence
  synthetic construct
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccaaggcctactagtaacggccgccagtgtgctggaattcgcccttggtacctgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacgatatcatggcggcggcggttcggatgaacatccagatgctgctggaggcggccgactatctggagcggcgggagagagaagctgaacatggttatgcctccatgttaccatacccgaaaaagaaacgtaaagtggggctcgagcccggggagaagccatataaatctcccgagtccggcaagtccttttctagatcagataatttagtaagacatcagagaacgcacaccggggagaagccgtacaagagccctgaatctggtaagtcattttcgagaagtgatgaattagtaagacaccagcggactcataccggggaaaagccctacaagagcccggaaagcggcaagtcttttagcaccagcggacatttagtaagacaccagagaacccacaccggggagaagccttataagtcccctgagagcggcaaaagcttcagcgatcctggaaatttagtaagacaccaacgcacccacaccggggaaaaaccttacaagtctcctgagagcggcaagagcttctctcaatcaagttcattagtaagacaccagaggactcataccggggagaaaccatacaagtccccagagagcgggaaaaactttagtacaagcggtgagttagtaagacaccaacgaacacacaccggtggatccggcggcagcggcggcagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttcgaatttaaatcggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatctgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttatgcgatcgagggcagaggaagtcttctaacatgcggtgacgtggaggagaatcccggccctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaaatcaacctctggattacaaaatttgtgaaagattgactggtattcttaactatgttgctccttttacgctatgtggatacgctgctttaatgcctttgtatcagttaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggtgtacactactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctctcgagatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 189 - DNA, 5185 bp, Artificial Sequence
  Synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcaccaccaccaccaccacggctatggccgcaaaaaacgccgccagcgccgccgcggctatccgtatgatgtgccggattatgccccatgggatatcggggatccgaattctgtacaggccttggcgcgcctgcaggcgagctccgtcgacaagcttgcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

(19) United States
(12) Patent Application Publication
(10) Pub. No.: US 2016/0039892 A1
(43) Pub. Date: Feb. 11, 2016

(54) ARTIFICIAL TRANSCRIPTION FACTORS AND THEIR USE FOR THE TREATMENT OF MALADAPTED WOUND HEALING IN THE EYE

(71) Applicant: ALIOPHTHA AG, Basel (CH)

(72) Inventors: Albert NEUTZNER, Schliengen (DE); Josef FLAMMER, Binningen (CH); Alice HUXLEY, Binningen (CH)

(73) Assignee: ALIOPHTHA AG, Basel (CH)

(21) Appl. No.: 14/781,694

(22) PCT Filed: Apr. 2, 2014
(86) PCT No.: PCT/EP2014/056597

§ 371 Date: Oct. 1, 2015

(30) Foreign Application Priority Data
Apr. 3, 2013 (EP) 13162203.7

(51) Int. Cl. C07K 14/435; A61K 38/17; A61K 47/48

(52) U.S. Cl. CPC C07K 14/435; A61K 47/48215; A61K 38/17; C07K 2319/09; C07K 2319/81

(57) ABSTRACT

The invention relates to artificial transcription factors comprising polydactyl zinc finger proteins targeting promoters of genes involved in maladapted wound healing in the eye. Such artificial transcription factors are useful for the treatment of fibrocontractive retinal disorders, such asepiretinal gliosis, proliferative vitreoretinopathy, proliferative diabetic retinopathy and epiretinal membrane, and for the treatment of fibroplasia associated with glaucoma surgery.

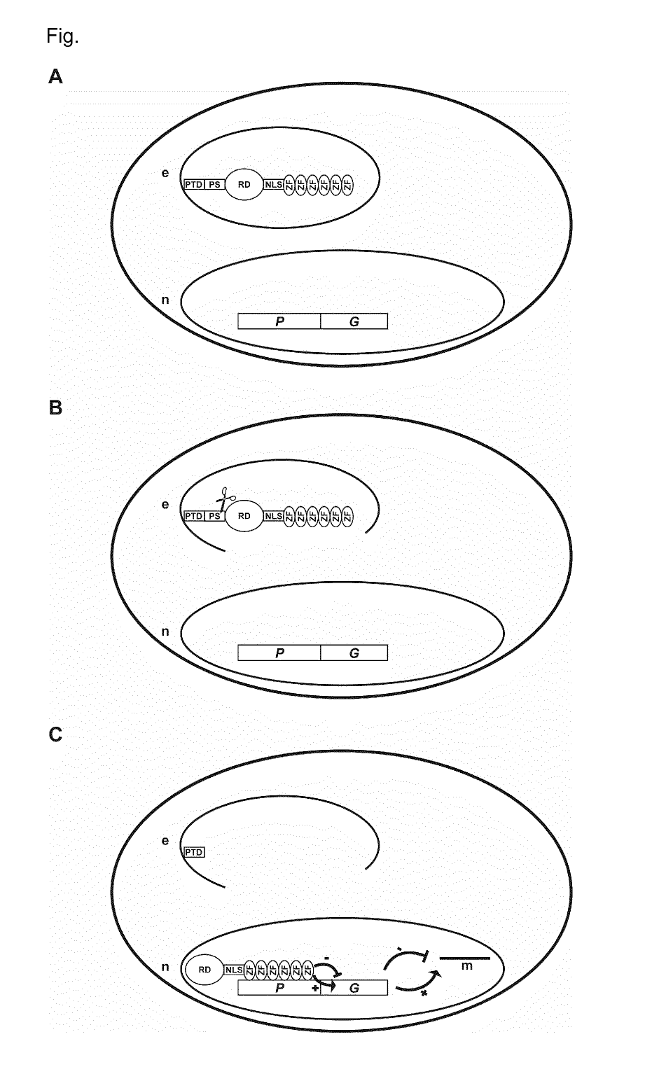

### FIELD OF THE INVENTION

The invention relates to artificial transcription factors comprising polydactyl zinc finger proteins targeting promoters of genes involved in maladapted wound healing in the eye, and to a method of modulating wound healing processes in the eye using such artificial transcription factors comprising a polydactyl zinc finger protein targeting specifically a disease-related gene promoter fused to an inhibitory or activatory domain, a nuclear localization sequence, a protein transduction domain and an endosome-specific protease-recognition site.

### BACKGROUND OF THE INVENTION

Wound healing through injury-induced proliferation of various cell types resulting in the formation of a scar is a necessary process for maintaining or restoring organ function in response to mechanical or immunological damage. However, maladapted wound healing can result in scar formation only partly restoring, or even interfering with, proper organ function. Such maladapted wound repair is often encountered in the eye, an exquisite organ relying on the correct interplay of many different cell types in a very ordered manner, biochemically, but also mechanically, optically and spatially. Thus, scar formation in the eye through maladapted wound repair is a major factor for vision impairment in patients.

Fibrocontractive retinal disorders such as epiretinal gliosis, proliferative vitreoretinopathy, proliferative diabetic retinopathy and epiretinal membrane are diseases of the eye caused by maladapted retinal wound repair, for example in response to vitreo-retinal surgery, diabetic alterations, or hypoxic damage. Furthermore, such maladapted wound healing is also associated with aging and the associated shrinkage of the vitreous, but also idiopathic fibrocontractive retinal disorders are observed. Clinical consequences are macular pucker, macular edema, retinal distortion, retinal detachment and ultimately blindness. Standard care for these diseases is unspecific anti-inflammatory treatment using highly dosed steroids or surgical removal of the epiretinal membrane or membrane peeling. Activation of various cell types of the retina including glial and Mueller cells, immune cells, (fibrious) astrocytes, pigment epithelial cells and microglia causes the formation of a transparent cellular layer covering the retina in early stages of the disease.

In late stage disease, this transparent cell layer starts to tighten and contract, at first distorting the retina and finally leading to retinal detachment and breakage. The activation of retinal cells to form this transparent layer is governed by a host of growth factors and their associated growth factor receptors as well as other factors connected to tissue remodeling and inflammation. Factors connected to fibrocontractive retinal disorders are hepatocyte growth factor (HGF), platelet-derived growth factor (PDGF), vascular endothelial growth factor (VEGF), pigment epithelium-derived factor (PEDF), transforming growth factor (TGF)-beta, epidermal growth factor (EGF), heparin-binding EGF-like growth factor (HBEGF), insulin-like growth factor 1 (IGF-1), connective tissue growth factor (CTGF), basic fibroblast growth factors (bFGF), tumor necrosis factor-alpha (TNF-alpha), but also matrix metallopeptidases 2 and 9 (MMP2, MMP9), and tissue inhibitor of MMPs 2 (TIMP2) (Moysidis S. N. et al., 2012, Mediators Inflamm, 2012:815937).

Glaucoma affects over 60 million people worldwide and is the second most blinding disease. Standard care for glaucoma associated with elevated intraocular pressure is pressure-lowering medication or eye surgery. Glaucoma filtration surgery is the gold standard for the management of intraocular pressure when medication and laser surgery have proven insufficient. However, scar tissue due to increased fibroblast proliferation or fibroplasia can form and obstruct aqueous flow causing the filter to fail. The antimetabolites mitomycin-C and 5-fluorouracil (locally applied during surgery and injected after surgery into the bleb) are used in current clinical practice to help limit post-operative ocular scar tissue formation. While these agents have been shown to improve surgery outcome, they do so in a non-selective manner. As a result, antimetabolite treatment is associated with a significant side-effect profile, including hypotony, blebitis, endophthalmitis, bleb leakage, and others.

Recent studies of alternative methods of preventing tissue fibrosis have focused on the inhibition of fibroblast proliferation, particularly through the regulation of various growth factors. Some success in reducing scar formation by inhibiting VEGF and TGF-beta has been reported in recent literature (Lockwood A. et al., 2013, Curr Opin Pharmacol 13 (1), 65-71). It was demonstrated, that the transdifferentiation of human Tenon's capsule fibroblasts to myofibroblasts is one of the most crucial events for scar formation and tissue remodeling after the surgery with TGF-beta is essential for this transdifferentiation. The TGF family shares similar abilities in regulating cell functions, such as proliferation, differentiation, apoptosis, and production of extracellular matrix. In the eye, the aqueous humor (flowing into the bleb) contains abundant TGF-beta2, whereas TGF-beta1 and -beta2 are expressed locally in the cells of the filtering bleb.

Modulation of gap junction communication following surgery is another possibility to reduce scar formation. Gap junctions are structures that allow direct signaling between cells. Six connexin protein subunits oligomerize to form a hemichannel called a connexon; two connexons from neighbouring cells dock to form a complete intercellular junction channel. Gap junctions play a role in inflammation, cell migration and tissue contraction. Rodent studies demonstrate that a transient reduction in connexin 43 protein expression is beneficial in skin wound healing and scar reduction (Deva N. C. et al., 2012, Inflammation 35 (4), 1276-86).

Artificial transcription factors (ATFs) are proposed to be useful tools for modulating gene expression (Sera T., 2009, Adv Drug Deliv Rev 61, 513-526). Many naturally occurring transcription factors, influencing expression either through repression or activation of gene transcription, possess complex specific domains for the recognition of a certain DNA sequence. This makes them unattractive targets for manipulation if one intends to modify their specificity and target gene(s). However, a certain class of transcription factors contains several so called zinc finger (ZF) domains, which are modular and therefore lend themselves to genetic engineering. Zinc fingers are short (30 amino acids) DNA binding motifs targeting almost independently three DNA base pairs. A protein containing several such zinc fingers fused together is thus able to recognize longer DNA sequences. A hexameric zinc finger protein (ZFP) recognizes an 18 base pairs (bp) DNA target, which is almost unique in the entire human genome. Initially thought to be completely context independent, more in-depth analyses revealed some context specificity for zinc fingers (Klug A., 2010, Annu Rev Biochem 79, 213-231). Mutating certain amino acids in the zinc finger recognition surface altering the binding specificity of ZF modules resulted in defined ZF building blocks for most of 5′-GNN-3′, 5′-CNN-3′, 5′-ANN-3′, and some 5′-TNN-3′ codons (e.g. so-called Barbas modules, see Dreier B., Barbas C. F. 3^{rd} et al., 2005, J Biol Chem 280, 35588-35597). While early work on artificial transcription factors concentrated on a rational design based on combining preselected zinc fingers with a known 3 bp target sequence, the realization of a certain context specificity of zinc fingers necessitated the generation of large zinc finger libraries which are interrogated using sophisticated methods such as bacterial or yeast one hybrid, phage display, compartmentalized ribosome display or in vivo selection using FACS analysis.

Using such artificial zinc finger proteins, DNA loci within the human genome can be targeted with high specificity. Thus, these zinc finger proteins are ideal tools to transport protein domains with transcription-modulatory activity to specific promoter sequences resulting in the modulation of expression of a gene of interest. Suitable domains for the silencing of transcription are the Krueppel-associated domain (KRAB) as N-Terminal (SEQ ID NO: 1) or C-terminal (SEQ ID NO: 2) KRAB domain, the Sin3-interacting domain (SID, SEQ ID NO: 3) and the ERF repressor domain (ERD, SEQ ID NO: 4), while activation of gene transcription is achieved through herpes virus simplex VP16 (SEQ ID NO: 5) or VP64 (tetrameric repeat of VP16, SEQ ID NO: 6) domains (Beerli R. R. et al., 1998, Proc Natl Acad Sci USA 95, 14628-14633). Additional domains considered to confer transcriptional activation are CJ7 (SEQ ID NO: 7), p65-TA1 (SEQ ID NO: 8), SAD (SEQ ID NO: 9), NF-1 (SEQ ID NO: 10), AP-2 (SEQ ID NO: 11), SP1-A (SEQ ID NO: 12), SP1-B (SEQ ID NO: 13), Oct-1 (SEQ ID NO: 14), Oct-2 (SEQ ID NO: 15), Oct-2_{—}5x (SEQ ID NO: 16), MTF-1 (SEQ ID NO: 17), BTEB-2 (SEQ ID NO: 18) and LKLF (SEQ ID NO: 19). In addition, transcriptionally active domains of proteins defined by gene ontology GO: 0001071 (http://amigo.geneontology.org/cgi-bin/amigo/term_details?term=GO:0001071) are considered to achieve transcriptional regulation of target proteins.

So called protein transduction domains (PTDs) were shown to promote protein translocation across the plasma membrane into the cytosol/nucleoplasm. Short peptides such as the HIV derived TAT peptide (SEQ ID NO: 20), mT02 (SEQ ID NO: 21), mT03 (SEQ ID NO: 22), R9 (SEQ ID NO: 23), ANTP (SEQ ID NO: 24) and others were shown to induce a cell-type independent macropinocytotic uptake when fused to cargo proteins (Wadia J. S. et al., 2004, Nat Med 10, 310-315). Upon arrival in the cytosol, such fusion proteins were shown to have biological activity. Interestingly, even misfolded proteins can become functional following protein transduction most likely through the action of intracellular chaperones. However, a major hurdle for the use of protein transduction domains for delivering therapeutic cargo to cells is the limited escape of such proteins from the endosomal compartment to other subcellular localization such as the nucleus (Koren E and Torchilin V. P., 2012, Trends in Mol Med 18, 385-393).

### SUMMARY OF THE INVENTION

The invention relates to an artificial transcription factor comprising a polydactyl zinc finger protein targeting specifically a promoter region of a gene involved in maladapted wound healing in the eye fused to an inhibitory or activatory protein domain, a nuclear localization sequence, a protein transduction domain, and optionally an endosome-specific protease-recognition site, and to pharmaceutical compositions comprising such an artificial transcription factor.

Furthermore the invention relates to the use of artificial transcription factors of the invention for increasing or decreasing the expression of genes involved in maladapted wound healing in the eye, and in treating diseases caused or influenced by such genes.

Likewise the invention relates to a method of treating a disease caused or modulated by genes involved in maladapted wound healing in the eye comprising administering a therapeutically effective amount of an artificial transcription factor of the invention to a patient in need thereof.

In a particular embodiment of the invention, the promoter region of the gene involved in maladapted wound healing in the eye is the AGER (RAGE) promoter (SEQ ID NO: 25). In this particular embodiment the invention relates to such an artificial transcription factor targeting the AGER promoter for use in influencing the activity of AGER through lowering or increasing AGER levels, and for use in the treatment of diseases modulated by this protein. Likewise the invention relates to a method of treating a disease modulated by AGER comprising administering a therapeutically effective amount of an artificial transcription factor of the invention targeting the AGER promoter to a patient in need thereof.

In another particular embodiment, the promoter region of the gene involved in maladapted wound healing in the eye is the EGFR promoter (SEQ ID NO: 26). In this particular embodiment the invention relates to such an artificial transcription factor targeting the EGFR promoter for use in influencing the activity of EGFR through lowering or increasing EGFR levels, and for use in the treatment of diseases modulated by this protein. Likewise the invention relates to a method of treating a disease modulated by EGFR comprising administering a therapeutically effective amount of an artificial transcription factor of the invention targeting the EGFR promoter to a patient in need thereof.

In another particular embodiment, the promoter region of the gene involved in maladapted wound healing in the eye is the FGFR1 promoter (SEQ ID NO: 27). In this particular embodiment the invention relates to such an artificial transcription factor targeting the FGFR1 promoter for use in influencing the activity of FGFR1 through lowering or increasing FGFR1 levels, and for use in the treatment of diseases modulated by this protein. Likewise the invention relates to a method of treating a disease modulated by FGFR1 comprising administering a therapeutically effective amount of an artificial transcription factor of the invention targeting the FGFR1 promoter to a patient in need thereof.

In another particular embodiment, the promoter region of the gene involved in maladapted wound healing in the eye is the FGFR2 promoter (SEQ ID NO: 28). In this particular embodiment the invention relates to such an artificial transcription factor targeting the FGFR2 promoter for use in influencing the activity of FGFR2 through lowering or increasing FGFR2 levels, and for use in the treatment of diseases modulated by this protein. Likewise the invention relates to a method of treating a disease modulated by FGFR2 comprising administering a therapeutically effective amount of an artificial transcription factor of the invention targeting the FGFR2 promoter to a patient in need thereof.

In another particular embodiment, the promoter region of the gene involved in maladapted wound healing in the eye is the FGFR3 promoter (SEQ ID NO: 29). In this particular embodiment the invention relates to such an artificial transcription factor targeting the FGFR3 promoter for use in influencing the activity of FGFR3 through lowering or increasing FGFR3 levels, and for use in the treatment of diseases modulated by this protein. Likewise the invention relates to a method of treating a disease modulated by FGFR3 comprising administering a therapeutically effective amount of an artificial transcription factor of the invention targeting the FGFR3 promoter to a patient in need thereof.

In another particular embodiment, the promoter region of the gene involved in maladapted wound healing in the eye is the FGFR4 promoter (SEQ ID NO: 30). In this particular embodiment the invention relates to such an artificial transcription factor targeting the FGFR4 promoter for use in influencing the activity of FGFR4 through lowering or increasing FGFR4 levels, and for use in the treatment of diseases modulated by this protein. Likewise the invention relates to a method of treating a disease modulated by FGFR4 comprising administering a therapeutically effective amount of an artificial transcription factor of the invention targeting the FGFR4 promoter to a patient in need thereof.

In another particular embodiment, the promoter region of the gene involved in maladapted wound healing in the eye is the FLT1 (VEGFR-1) promoter (SEQ ID NO: 31). In this particular embodiment the invention relates to such an artificial transcription factor targeting the FLT1 promoter for use in influencing the activity of FLT1 through lowering or increasing FLT1 levels, and for use in the treatment of diseases modulated by this protein. Likewise the invention relates to a method of treating a disease modulated by FLT1 comprising administering a therapeutically effective amount of an artificial transcription factor of the invention targeting the FLT1 promoter to a patient in need thereof.

In another particular embodiment, the promoter region of the gene involved in maladapted wound healing in the eye is the FLT4 (VEGFR-3) promoter (SEQ ID NO: 32). In this particular embodiment the invention relates to such an artificial transcription factor targeting the FLT4 promoter for use in influencing the activity of FLT4 through lowering or increasing FLT4 levels, and for use in the treatment of diseases modulated by this protein. Likewise the invention relates to a method of treating a disease modulated by FLT4 comprising administering a therapeutically effective amount of an artificial transcription factor of the invention targeting the FLT4 promoter to a patient in need thereof.

In another particular embodiment, the promoter region of the gene involved in maladapted wound healing in the eye is the GJA1 (CX43) promoter (SEQ ID NO: 33). In this particular embodiment the invention relates to such an artificial transcription factor targeting the GJA1 promoter for use in influencing the activity of GJA1 through lowering or increasing GJA1 levels, and for use in the treatment of diseases modulated by this protein. Likewise the invention relates to a method of treating a disease modulated by GJA1 comprising administering a therapeutically effective amount of an artificial transcription factor of the invention targeting the GJA1 promoter to a patient in need thereof.

In another particular embodiment, the promoter region of the gene involved in maladapted wound healing in the eye is the IGF1R promoter (SEQ ID NO: 34). In this particular embodiment the invention relates to such an artificial transcription factor targeting the IGF1R promoter for use in influencing the activity of IGF1R through lowering or increasing IGF1R levels, and for use in the treatment of diseases modulated by this protein. Likewise the invention relates to a method of treating a disease modulated by IGF1R comprising administering a therapeutically effective amount of an artificial transcription factor of the invention targeting the IGF1R promoter to a patient in need thereof.

In another particular embodiment, the promoter region of the gene involved in maladapted wound healing in the eye is the KDR (VEGFR-2) promoter (SEQ ID NO: 35). In this particular embodiment the invention relates to such an artificial transcription factor targeting the KDR promoter for use in influencing the activity of KDR through lowering or increasing KDR levels, and for use in the treatment of diseases modulated by this protein. Likewise the invention relates to a method of treating a disease modulated by KDR comprising administering a therapeutically effective amount of an artificial transcription factor of the invention targeting the KDR promoter to a patient in need thereof.

In another particular embodiment, the promoter region of the gene involved in maladapted wound healing in the eye is the MET (HGFR) promoter (SEQ ID NO: 36). In this particular embodiment the invention relates to such an artificial transcription factor targeting the MET promoter for use in influencing the activity of MET through lowering or increasing MET levels, and for use in the treatment of diseases modulated by this protein. Likewise the invention relates to a method of treating a disease modulated by MET comprising administering a therapeutically effective amount of an artificial transcription factor of the invention targeting the MET promoter to a patient in need thereof.

In another particular embodiment, the promoter region of the gene involved in maladapted wound healing in the eye is the PDGFRA promoter (SEQ ID NO: 37). In this particular embodiment the invention relates to such an artificial transcription factor targeting the PDGFRA promoter for use in influencing the activity of PDGFRA through lowering or increasing PDGFRA levels, and for use in the treatment of diseases modulated by this protein. Likewise the invention relates to a method of treating a disease modulated by PDGFRA comprising administering a therapeutically effective amount of an artificial transcription factor of the invention targeting the PDGFRA promoter to a patient in need thereof.

In another particular embodiment, the promoter region of the gene involved in maladapted wound healing in the eye is the PDGFRB promoter (SEQ ID NO: 38). In this particular embodiment the invention relates to such an artificial transcription factor targeting the PDGFRB promoter for use in influencing the activity of PDGFRB through lowering or increasing PDGFRB levels, and for use in the treatment of diseases modulated by this protein. Likewise the invention relates to a method of treating a disease modulated by PDGFRB comprising administering a therapeutically effective amount of an artificial transcription factor of the invention targeting the PDGFRA promoter to a patient in need thereof.

In another particular embodiment, the promoter region of the gene involved in maladapted wound healing in the eye is the PNPLA2 (PEDF-R) promoter (SEQ ID NO: 39). In this particular embodiment the invention relates to such an artificial transcription factor targeting the PNPLA2 promoter for use in influencing the activity of PNPLA2 through lowering or increasing PNPLA2 levels, and for use in the treatment of diseases modulated by this protein. Likewise the invention relates to a method of treating a disease modulated by PNPLA2 comprising administering a therapeutically effective amount of an artificial transcription factor of the invention targeting the PNPLA2 promoter to a patient in need thereof.

In another particular embodiment, the promoter region of the gene involved in maladapted wound healing in the eye is the TGFBR1 promoter (SEQ ID NO: 40). In this particular embodiment the invention relates to such an artificial transcription factor targeting the TGFBR1 promoter for use in influencing the activity of TGFBR1 through lowering or increasing TGFBR1 levels, and for use in the treatment of diseases modulated by this protein. Likewise the invention relates to a method of treating a disease modulated by TGFBR1 comprising administering a therapeutically effective amount of an artificial transcription factor of the invention targeting the TGFBR1 promoter to a patient in need thereof.

In another particular embodiment, the promoter region of the gene involved in maladapted wound healing in the eye is the TGFBR2 promoter (SEQ ID NO: 41). In this particular embodiment the invention relates to such an artificial transcription factor targeting the TGFBR2 promoter for use in influencing the activity of TGFBR2 through lowering or increasing TGFBR2 levels, and for use in the treatment of diseases modulated by this protein. Likewise the invention relates to a method of treating a disease modulated by TGFBR2 comprising administering a therapeutically effective amount of an artificial transcription factor of the invention targeting the TGFBR2 promoter to a patient in need thereof.

In another particular embodiment, the promoter region of the gene involved in maladapted wound healing in the eye is the TGFBR3 promoter (SEQ ID NO: 42). In this particular embodiment the invention relates to such an artificial transcription factor targeting the TGFBR3 promoter for use in influencing the activity of TGFBR3 through lowering or increasing TGFBR3 levels, and for use in the treatment of diseases modulated by this protein. Likewise the invention relates to a method of treating a disease modulated by TGFBR3 comprising administering a therapeutically effective amount of an artificial transcription factor of the invention targeting the TGFBR3 promoter to a patient in need thereof.

In another particular embodiment, the promoter region of the gene involved in maladapted wound healing in the eye is the TNFRSF1A promoter (SEQ ID NO: 43). In this particular embodiment the invention relates to such an artificial transcription factor targeting the TNFRSF1A promoter for use in influencing the activity of TNFRSF1A through lowering or increasing TNFRSF1A levels, and for use in the treatment of diseases modulated by this protein. Likewise the invention relates to a method of treating a disease modulated by TNFRSF1A comprising administering a therapeutically effective amount of an artificial transcription factor of the invention targeting the TNFRSF1A promoter to a patient in need thereof.

In another particular embodiment, the promoter region of the gene involved in maladapted wound healing in the eye is the TNFRSF1B promoter (SEQ ID NO: 44). In this particular embodiment the invention relates to such an artificial transcription factor targeting the TNFRSF1B promoter for use in influencing the activity of TNFRSF1B through lowering or increasing TNFRSF1B levels, and for use in the treatment of diseases modulated by this protein. Likewise the invention relates to a method of treating a disease modulated by TNFRSF1B comprising administering a therapeutically effective amount of an artificial transcription factor of the invention targeting the TNFRSF1B promoter to a patient in need thereof.

Furthermore the invention relates to the mentioned artificial transcription factors for the treatment of fibrocontractive retinal disorders, such as epiretinal gliosis, proliferative vitreoretinopathy, proliferative diabetic retinopathy and epiretinal membrane, fibroplasia following glaucoma surgery, and to a method of treating fibroplasia and fibrocontractive retinal disorders comprising administering a therapeutically effective amount of an artificial transcription factor of the invention to a patient in need thereof.

The invention further relates to nucleic acids coding for an artificial transcription factor of the invention, vectors comprising these, and host cells comprising such vectors.

### BRIEF DESCRIPTION OF THE FIGURE

The FIGURE is a schematic representation of modulating gene expression using protease-sensitive transducible artificial transcription factors.

An artificial transcription factor comprising a protein transduction domain (PTD), an endosome-specific protease cleavage site (PS), a domain with transcription regulating activity (RD), a nuclear localization sequence (NLS), and a polydactyl zinc finger (ZF) protein specific for the promoter region (P) of a gene (G) enters the cell via an endocytotic mechanism. In (A) such an artificial transcription factor is trapped inside the endosomal compartment (e) unable to reach efficiently the nucleus (n). In (B), an endosome-specific protease (symbolized by scissors) is activated during endosomal maturation, recognizes PS and cleaves the artificial transcription factor, thus separating PTD from RD-NLS-ZFₙ. Following rupture of the endosomal vesicle, the now cleaved artificial transcription factor is able to leave the endosomal compartment and is being transported to the nucleus, see (C). Upon binding to its target site in the promoter region P of gene G, production of mRNA (m) is either up- or downregulated (+ or −), depending on the transcription regulating activity of the regulatory domain RD.

### DETAILED DESCRIPTION OF THE INVENTION

The invention relates to an artificial transcription factor comprising a polydactyl zinc finger protein targeting specifically the promoter of a gene involved in maladapted wound healing in the eye, fused to an inhibitory or activatory protein domain, a nuclear localization sequence, a protein transduction domain, and optionally an endosome-specific protease-recognition site, and to pharmaceutical compositions comprising such an artificial transcription factor. Furthermore the invention relates to the use of such artificial transcription factors for modulating the expression of gene involved in maladapted wound healing in the eye, and in treating diseases caused or modulated by such genes.

In the context of this invention, a promoter is defined as the regulatory region of a gene as well known in the art. Again in this context, a gene is defined, as well known in the art, as genomic region containing regulatory sequences as well as sequences for the gene product resulting in the production of proteins or RNAs. In this context, a gene involved in maladapted wound healing in the eye is a gene whose gene product is actively involved in modulating cell growth at the site of tissue damage in the eye or whose gene product is accidently active even in the absence of injury, and promotes excess cell growth or scar formation.

In the context of the invention, an endosome-specific protease-recognition site is a peptide sequence that is recognized and cleaved in a sequence-specific manner by proteases resident to the endosomal compartment. Again in the context of this invention, a protein transduction domain is defined as a peptide capable of transporting proteins such as artificial transcription factors across the plasma membrane into the intracellular compartment.

In the context of the present invention, a polydactyl zinc finger protein targeting “specifically” a gene promoter has a binding affinity of 20 nM or less towards its DNA target.

Genes considered in the present invention as involved in maladapted wound healing in the eye are AGER (RAGE), EGFR, FGFR1, FGFR2, FGFR3, FGFR4, FLT1 (VEGFR1), FLT4 (VEGFR-3), GJA1 (CX43), IGF1R, KDR (VEGFR-2), MET (HGFR), PDGFRA, PDGFRB, PNPLA2 (PEDF-R), TGFBR1, TGFBR2, TGFBR3, TNFRSF1A, and TNFRSF1B.

Artificial transcription factors are useful for modulating gene expression, and thus are useful for the treatment of diseases wherein the modulation of gene expression is beneficial. While conventional drugs modulate the activity of a certain protein, e.g. by agonistic or antagonistic action, artificial transcription factors alter the availability of these proteins either by increasing or decreasing gene expression.

Using the traditional small molecule approach, the identification of therapeutically active small molecules acting through modulation of protein activity mostly relies on extensive and time-consuming screening procedures among a wide variety of different molecules from different classes of substances and modulation of gene expression by small molecules is so far not possible. In contrast, artificial transcription factors of the invention all belong to the same substance class with a highly defined overall composition. Two hexameric zinc finger protein-based artificial transcription factors targeting two very diverse promoter sequences still have a minimal amino acid sequence identity of 85% with an overall similar tertiary structure and can be generated via a standardized method (as described below) in a fast and economical manner. Thus, artificial transcription factors of the invention combine, in one class of molecule, exceptionally high specificity for a very wide and diverse set of targets with overall similar composition. As for all biologicals, immunogenicity in the form of anti-drug antibodies and the associated immunological reaction are a concern. However, due to the high conservation of zinc finger modules such an immunological reaction will be minor or absent following application of artificial transcription factors of the invention, or might be avoided or further minimized by small changes to the overall structure eliminating immunogenicity while still retaining target site binding and thus function. Furthermore, modification of artificial transcription factors of the invention with polyethylene glycol is considered to reduce immunogenicity.

Since artificial transcription factors are tailored to act specifically on the promoter region of specific genes, the use of artificial transcription factors allows for selectively targeting even closely related proteins. This is based on the only loose conservation of the promoter regions even of closely related proteins. Taking advantage of the high selectivity of the artificial transcription factors according to the invention, even a tissue-specific targeting of a drug action is possible based on the oftentimes tissue-specific expression of certain members of a given protein family that are individually addressable using artificial transcription factors.

In addition, formulation of artificial transcription factors into drugs can rely on previous experience further expediting the drug development process.

However, artificial transcription factors need to be present in the nuclear compartment of cells in order to be effective as they act through modulation of gene expression. Until now, the method of choice for the therapeutic delivery of artificial transcription factors is either in the form of plasmid DNA through transfection or by employing viral vectors. Plasmid transfection for therapeutic purposes has low efficacy, while viral vectors have exceptionally high potential for immunogenicity, thus limiting their use in repeated application of a certain treatment. Thus other modes of delivering artificial transcription factors for example in protein form instead of as nuclei acid are required.

Protein transduction domain (PTD) mediated, intracellular delivery of artificial transcription factors is a new way of taking advantage of the high selectivity and versatility of artificial transcription factors in a novel fashion. Protein transduction domains are small peptides capable of crossing the plasma membrane barrier and delivering cargo proteins into the cell. Such protein transduction domains are for example the HIV derived TAT peptide, mT02, mT03, R9, ANTP and others. The mode of cellular uptake is likely by endocytosis and it was shown that the TAT peptide is able to induce a cell-type independent macropinocytotic uptake when fused to cargo proteins (Wadia J. S. et al., 2004, Nat Med 10, 310-315). While crossing the barrier of the plasma membrane and uptake into endosomal vesicles is the first step in entering the cell, topologically, the inside of the endosomal compartment is identical to the outside of the cell. Thus, endosomal localization is not equivalent to cytoplasmic or nucleoplasmic localization. However, likely through leakiness of the endosomal compartment and/or some intrinsic property of the cargo or the protein transduction domain in terms of modulating membrane integrity, delivered proteins are capable to escape endosomes and reach other truly intracellular targets. The co-delivery of the membrane-active, fusogenic peptides TAT-HA2 or others such as GALA or KALA peptide improved endosomal escape of delivered proteins somewhat due to the disintegration of endosomal vesicles.

However, fusogenic peptides are not as efficient in promoting delivery as expected. This might be due to the inherent properties of protein transduction domains. Protein transduction domains are known to strongly interact with cellular membranes. This strong membrane interaction is part of the mechanism by which protein internalization and thus protein delivery is triggered. Thus, following internalization into endosomes, this strong membrane-interaction of the protein transduction domain now with the inside of the endosomal membrane might actually inhibit redistribution even after the rupture of endosomal vesicles. TAT-fused artificial transcription factors may mainly reside in the endosomal compartment with some nuclear localization. Interestingly, in a large percentage of cells stained for TAT-artificial transcription factor, ruptured endosomal vesicles can be found open to the cytosol with endosomal membranes clearly decorated with TAT fusion protein consistent with endosomal entrapment of a considerable amount of delivered protein even after endosomal membrane rupture. Thus, while essential for uptake into the cell, protein transduction domains may hinder efficient subcellular localization once protein transduction took place.

The endosome is a very dynamic organelle known to mature and acquire lysosomal characteristic, such as acquiring proteases and indicating a drop in vesicular pH before fusion with the lysosomal compartment and proteolytic degradation of the endosomal content. The process of endosomal maturation accompanied by an increase in lumenal proteolytic activity is detrimental for therapeutic proteins delivered using protein transduction domains, since such proteins are then subject to proteolysis. However, this process can be turned into an advantage. Endosomal maturation is a sequential process wherein different sets of proteases are activated at different stages in a pH-dependent manner. Interestingly, proteases activated early in the process involved in protein processing are more sequence specific than proteases activated late during maturation essential for general hydrolysis of proteins. Now, incorporation of a cleavage site for an early endosomal protease between the protein transduction domain and the cargo protein leads to the sequence-specific digestion of the therapeutic protein separating the protein transduction domain from the cargo protein once the therapeutic protein has reached the endosomal lumen. Thus, upon endosomal rupture, frequently observable following TAT-mediated delivery of artificial transcription factors, the cargo protein is no longer bound to the inside of the endosomal membrane due to inherent properties of the protein transduction domain but is detached from the membrane in order to escape into the cytosol.

Further, the artificial transcription factors of the invention comprise a nuclear localization sequence (NLS). Nuclear localization sequences considered are amino acid motifs conferring nuclear import through binding to proteins defined by gene ontology GO:0008139, for example clusters of basic amino acids containing a lysine residue (K) followed by a lysine (K) or arginine residue (R), followed by any amino acid (X), followed by a lysine or arginine residue (K-K/R-X-K/R consensus sequence, Chelsky D. et al., 1989 Mol Cell Biol 9, 2487-2492) or the SV40 NLS (SEQ ID NO: 45), with the SV40 NLS being preferred.

The artificial transcription factor of the present invention might also contain other transcriptionally active protein domains of proteins defined by gene ontology GO:0001071 such as N-terminal KRAB, C-terminal KRAB, SID and ERD domains, preferably KRAB or SID. Activatory protein domains considered are the transcriptionally active domains of proteins defined by gene ontology GO:0001071, such as VP16 or VP64 (tetrameric repeat of VP16), CJ7, p65-TA1, SAD, NF-1, AP-2, SP1-A, SP1-B, Oct-1, Oct-2, Oct-2_{—}5x, MTF-1, BTEB-2 and LKLF, preferably VP64.

Further considered are alternative delivery methods for artificial transcription factors of the invention in form of nucleic acids transferred by transfection or via viral vectors such as herpes virus-, adeno virus- and adeno-associated virus-based vectors.

Considered are also artificial transcription factors of the invention containing pentameric, hexameric, heptameric or octameric zinc finger proteins where individual zinc finger modules are exchanged to improve binding affinity towards target sites of the respective nuclear receptor promoter gene or to alter the immunological profile of the zinc finger protein for improved tolerability.

The domains of the artificial transcription factors of the invention may be connected by short flexible linkers. A short flexible linker has 2 to 8 amino acids, preferably glycine and serine. A particular linker considered is GGSGGS (SEQ ID NO: 46). Artificial transcription factors may further contain markers, such as epitope tags, to ease their detection and processing.

Selection of Target Sites within a Given Promoter Region

Target site selection is crucial for the successful generation of a functional artificial transcription factor. For an artificial transcription factor to modulate target gene expression in vivo, it must bind its target site in the genomic context of the target gene. This necessitates the accessibility of the DNA target site, meaning chromosomal DNA in this region is not tightly packed around histones into nucleosomes and no DNA modifications such as methylation interfere with artificial transcription factor binding. While large parts of the human genome are tightly packed and transcriptionally inactive, the immediate vicinity of the transcriptional start site (−1000 to +200 bp) of an actively transcribed gene must be accessible for endogenous transcription factors and the transcription machinery such as RNA polymerases. Thus, selecting a target site in this area of any given target gene will greatly enhance the success rate for the generation of an artificial transcription factor with the desired function in vivo.

Selection of Target Sites within Promoters of Genes Involved in Maladapted Wound Healing in the Eye

The promoter region of genes involved in maladapted wound healing 2000 bp upstream and 500 bp downstream of the transcriptional start site was analyzed for the presence of potential 18 bp target sites with the general composition of (G/CANN)₆, wherein G is the nucleotide guanine, C the nucleotide cytosine, A the nucleotide adenine, and N stands for each of the four nucleotide guanine, cytosine, adenine and thymine. Two target sites in each promoter were selected based on their position relative to the transcription start site. These target sites are: AGER_TS1 (SEQ ID NO: 47), AGER_TS2 (SEQ ID NO: 48), EGFR_TS1 (SEQ ID NO: 49), EGFR_TS2 (SEQ ID NO: 50), FGFR1_TS1 (SEQ ID NO: 51), FGFR1_TS2 (SEQ ID NO: 52), FGFR2_TS1 (SEQ ID NO: 53), FGFR2_TS2 (SEQ ID NO: 54), FGFR3_TS1 (SEQ ID NO: 55), FGFR3_TS2 (SEQ ID NO: 56), FGFR4_TS1 (SEQ ID NO: 57), FGFR4_TS2 (SEQ ID NO: 58), FLT1_TS1 (SEQ ID NO: 59), FLT1_TS2 (SEQ ID NO: 60), FLT4_TS1 (SEQ ID NO: 61), FLT4_TS2 (SEQ ID NO: 62), GJA1_TS1 (SEQ ID NO: 63), GJA1_TS2 (SEQ ID NO: 64), IGF1R_TS1 (SEQ ID NO: 65), IGF1R_TS2 (SEQ ID NO: 66), KDR_TS1 (SEQ ID NO: 67), KDR_TS2 (SEQ ID NO: 68), MET_TS1 (SEQ ID NO: 69), MET_TS2 (SEQ ID NO: 70), PDGFRA_TS1 (SEQ ID NO: 71), PDGFRA_TS2 (SEQ ID NO: 72), PDGFRB_TS1 (SEQ ID NO: 73), PDGFRB_TS2 (SEQ ID NO: 74), PNPLA2_TS1 (SEQ ID NO: 75), PNPLA2_TS2 (SEQ ID NO: 76), TGFBR1_TS1 (SEQ ID NO: 77), TGFBR1_TS2 (SEQ ID NO: 78), TGFBR1_TS-390 (SEQ ID NO: 79), TGFBR2_TS1 (SEQ ID NO: 80), TGFBR2_TS2 (SEQ ID NO: 81), TGFBR3_TS1 (SEQ ID NO: 82), TGFBR3_TS2 (SEQ ID NO: 83), TNFRSF1A_TS1 (SEQ ID NO: 84), TNFRSF1A_TS2 (SEQ ID NO: 85), TNFRSF1B_TS1 (SEQ ID NO: 86), and TNFRSF1B_TS2 (SEQ ID NO: 87).

### Artificial Transcription Factors Targeting Promoters of Genes Involved in Maladapted Wound Healing in the Eye

Specific hexameric zinc finger proteins targeting specific target sites within promoters of genes involved in maladapted wound healing in the eye are composed of the Barbas zinc finger module set (Gonzalez B., 2010, Nat Protoc 5, 791-810) using the ZiFit software v3.3 (Sander J. D., Nucleic Acids Research 35, 599-605) or are selected using improved yeast one hybrid screening.

The hexameric zinc fingers specific for target sites within promoters of genes involved in maladapted wound healing in the eye are: AGER_{—}1 (SEQ ID NO: 88), AGER_{—}2 (SEQ ID NO: 89), EGFR_{—}1 (SEQ ID NO: 90), EGFR_{—}2 (SEQ ID NO: 91), FGFR1_{—}1 (SEQ ID NO: 92), FGFR1_{—}2 (SEQ ID NO: 93), FGFR2_{—}1 (SEQ ID NO: 94), FGFR2_{—}2 (SEQ ID NO: 95), FGFR3_{—}1 (SEQ ID NO: 96), FGFR3_{—}2 (SEQ ID NO: 97), FGFR4_{—}1 (SEQ ID NO: 98), FGFR4_{—}2 (SEQ ID NO: 99), FLT1_{—}1 (SEQ ID NO: 100), FLT1_{—}2 (SEQ ID NO: 101), FLT4_{—}1 (SEQ ID NO: 102), FLT4_{—}2 (SEQ ID NO: 103), GJA1_{—}1 (SEQ ID NO: 104), GJA1_{—}2 (SEQ ID NO: 105), IGF1R_{—}1 (SEQ ID NO: 106), IGF1R_{—}2 (SEQ ID NO: 107), KDR_{—}1 (SEQ ID NO: 108), KDR_{—}2 (SEQ ID NO: 109), MET_{—}1 (SEQ ID NO: 110), MET_{—}2 (SEQ ID NO: 111), PDGFRA_{—}1 (SEQ ID NO: 112), PDGFRA_{—}2 (SEQ ID NO: 113), PDGFRB_{—}1 (SEQ ID NO: 114), PDGFRB_{—}2 (SEQ ID NO: 115), PNPLA2_{—}1 (SEQ ID NO: 116), PNPLA2_{—}2 (SEQ ID NO: 117), TGFBR1_{—}1 (SEQ ID NO: 118), TGFBR1_{—}2 (SEQ ID NO: 119), TGRBR1-390B (SEQ ID NO: 120), TGFBR2_{—}1 (SEQ ID NO: 121), TGFBR2_{—}2 (SEQ ID NO: 122), TGFBR3_{—}1 (SEQ ID NO: 123), TGFBR3_{—}2 (SEQ ID NO: 124), TNFRSF1A_{—}1 (SEQ ID NO: 125), TNFRSF1A_{—}2 (SEQ ID NO: 126), TNFRSF1B_{—}1 (SEQ ID NO: 127), and TNFRSF1B_{—}2 (SEQ ID NO: 128).

To generate inhibiting, transducible artificial transcription factors, hexameric zinc finger proteins were fused to the protein transduction domain TAT as well as the transcription repressing domain SID yielding artificial transcription factors AGER_{—}1rep (SEQ ID NO: 129), AGER_{—}2rep (SEQ ID NO: 130), EGFR_{—}1rep (SEQ ID NO: 131), EGFR_{—}2rep (SEQ ID NO: 132), FGFR1_{—}1rep (SEQ ID NO: 133), FGFR1_{—}2rep (SEQ ID NO: 134), FGFR2_{—}1rep (SEQ ID NO: 135), FGFR2_{—}2rep (SEQ ID NO: 136), FGFR3_{—}1rep (SEQ ID NO: 137), FGFR3_{—}2rep (SEQ ID NO: 138), FGFR4_{—}1rep (SEQ ID NO: 139), FGFR4_{—}2rep (SEQ ID NO: 140), FLT1_{—}1rep (SEQ ID NO: 141), FLT1_{—}2rep (SEQ ID NO: 142), FLT4_{—}1rep (SEQ ID NO: 143), FLT4_{—}2rep (SEQ ID NO: 144), GJA1_{—}1rep (SEQ ID NO: 145), GJA1_{—}2rep (SEQ ID NO: 146), IGF1R_{—}1rep (SEQ ID NO: 147), IGF1R_{—}2rep (SEQ ID NO: 148), KDR_{—}1rep (SEQ ID NO: 149), KDR_{—}2rep (SEQ ID NO: 150), MET_{—}1rep (SEQ ID NO: 151), MET_{—}2rep (SEQ ID NO: 152), PDGFRA_{—}1rep (SEQ ID NO: 153), PDGFRA_{—}2rep (SEQ ID NO: 154), PDGFRB_{—}1rep (SEQ ID NO: 155), PDGFRB_{—}2rep (SEQ ID NO: 156), TGFBR1_{—}1rep (SEQ ID NO: 157), TGFBR1_{—}2rep (SEQ ID NO: 158), TGFBR1-390Brep (SEQ ID NO: 159), TGFBR2_{—}1rep (SEQ ID NO: 160), TGFBR2_{—}2rep (SEQ ID NO: 161), TGFBR3_{—}1rep (SEQ ID NO: 162), TGFBR3_{—}2rep (SEQ ID NO: 163), TNFRSF1A_{—}1rep (SEQ ID NO: 164), TNFRSF1A_{—}2rep (SEQ ID NO: 165), TNFRSF1B_{—}1rep (SEQ ID NO: 166), and TNFRSF1B_{—}2rep (SEQ ID NO: 167).

To generate activating, transducible artificial transcription factors targeting, hexameric zinc finger proteins were fused to the protein transduction domain TAT as well as the transcription activating domain VP64 yielding artificial transcription factors PNPLA2_{—}1akt (SEQ ID NO: 168) and PNPLA2_{—}2akt (SEQ ID NO: 169).

Considered are also artificial transcription factors of the invention containing pentameric, hexameric, heptameric or octameric zinc finger proteins, wherein individual zinc finger modules are exchanged to improve binding affinity towards target sites of the respective nuclear receptor promoter gene or to alter the immunological profile of the zinc finger protein for improved tolerability.

In another particular embodiment, the artificial transcription factors targeting promoters of genes involved in maladapted wound healing in the eye according to the invention comprise a zinc finger protein based on the zinc finger module composition of SEQ ID NO: 88 to 128, wherein up to three, preferably one or two, individual zinc finger modules are exchanged against other zinc finger modules with alternative binding characteristic to modulate the binding of the artificial transcription factor to its target sequence, and/or wherein up to twelve, for example twelve, eleven, ten or nine, in particular eight, seven, six or five, preferably four or three, most preferably one or two, individual amino acids are exchanged in order to minimize potential immunogenicity while retaining binding affinity to the intended target site.

In a particular embodiment, the artificial transcription factors targeting promoters of genes involved in maladapted wound healing in the eye comprise a zinc finger protein based on the zinc finger module composition of SEQ ID NO: 88 to 128, wherein optionally up to three, preferably one or two, individual zinc finger modules are exchanged against other zinc finger modules with alternative binding characteristic to modulate the binding of the artificial transcription factor to its target sequence, and/or wherein optionally up to twelve, most preferably one or two individual amino acids are exchanged in order to minimize potential immunogenicity while retaining binding affinity to the intended target site, and wherein the transcription modulating domain is VP16, VP64, N-KRAB, C-KRAB, SID or ERD.

### Activity of Artificial Transcription Factors in Regulating Receptor Promoter Activity

To assess the potential of artificial transcription factors to influence transcription driven by promoters of genes involved in maladapted wound healing in the eye, a luciferase reporter assay is employed. To this end, cells capable of driving expression from such promoters are co-transfected with an artificial transcription factor expression plasmid together with a dual-reporter plasmid. The dual-reporter plasmid contains the secreted Gaussia luciferase gene under the control of the gene promoter to be examined together with the gene for secreted alkaline phosphatase (SEAP) under control of the constitutive CMV promoter based on the NEG-PG04 and EF1a-PG04 plasmids (GeneCopoeia, Rockville, Md.). This co-transfection is done in a 3:1 artificial transcription factor expression plasmid:reporter plasmid ratio to ensure the presence of artificial transcription factor expression in cells transfected with the reporter plasmid, and Gaussia luciferase, and SEAP activity is measured according to manufacturer's recommendation (Gaussia Luciferase Glow Assay Kit, Pierce; SEAP Reporter Gene Assay Chemiluminescence, Roche). Luciferase values are normalized to SEAP activity and compared to control cells expressing an inactive variant of the artificial transcription factor where all cystein residues inside the zinc finger domains are exchanged to serine residues. By measuring the ratio between luciferase and SEAP activity in the supernatant of transfected cells, normalization of receptor promoter-driven luciferase expression to SEAP expression only in cells transfected with artificial transcription factor plasmid is possible. The luciferase expression studies are performed at least three times in triplicates, averaged, compared to control transfected cells, expressed as relative luciferase activity (RLuA) in % of control and plotted with error bars depicting SEM.

### Assessment of Artificial Transcription Factor Activity Towards Genes Involved in Maladapted Wound Healing in the Eye

In order to positively influence the maladapted wound healing process, the artificial transcription factors of the invention restrict cell growth following injury. In order to assess this function of the artificial transcription factors of the invention, cells expressing genes involved in maladapted wound healing are treated with specific artificial transcription factors of the invention and the rate of wound healing following injury is measured using electric cell-substrate impedance sensing (ECIS). To this end, cells are grown on gold electrodes in 96-well plates (Applied BioPhysics) and treated with artificial transcription factors for 0, 24, 48, 72 and 96 hours. Cells treated with an inactive variant of the artificial transcription factor serve as control. Using ECIS, the baseline impedance of the cell layer is measured at >40 kHz before injuring the cell layer by applying a short high voltage pulse. Following injury, the reformation of the cell layer is followed in real time using ECIS until control cells form a closed cell layer. Comparison between impedance measurements of treated cells with control cells reveals the cell growth restricting activity of the artificial transcription factor specific for the promoter of the gene involved in maladapted wound healing.

### Attachment of a Polyethylene Glycol Residue

The covalent attachment of a polyethylene glycol residue (PEGylation) to an artificial transcription factor of the invention is considered to increase solubility of the artificial transcription factor, to decrease its renal clearance, and control its immunogenicity. Considered are amine as well as thiol reactive polyethylene glycols ranging in size from 1 to 40 Kilodalton. Using thiol reactive polyethylene glycols, site-specific PEGylation of the artificial transcription factors is achieved. The only essential thiol group containing amino acids in the artificial transcription factors of the invention are the cysteine residues located in the zinc finger modules essential for zinc coordination. These thiol groups are not accessible for PEGylation due their zinc coordination, thus, inclusion of one or several cysteine residues into the artificial transcription factors of the invention provides free thiol groups for PEGylation using thiol-specific polyethylene glycol reagents.

### Pharmaceutical Compositions

The present invention relates also to pharmaceutical compositions comprising an artificial transcription factor as defined above. Pharmaceutical compositions considered are compositions for parenteral systemic administration, in particular intravenous administration, compositions for inhalation, and compositions for local administration, in particular ophthalmic-topical administration, e.g. as eye drops, or intravitreal, subconjunctival, parabulbar or retrobulbar administration, to warm-blooded animals, especially humans. Particularly preferred are eye drops and compositions for intravitreal, subconjunctival, parabulbar or retrobulbar administration. The compositions comprise the active ingredient alone or, preferably, together with a pharmaceutically acceptable carrier. Further considered are slow-release formulations. The dosage of the active ingredient depends upon the disease to be treated and upon the species, its age, weight, and individual condition, the individual pharmacokinetic data, and the mode of administration.

Further considered are pharmaceutical compositions useful for oral delivery, in particular compositions comprising suitably encapsulated active ingredient, or otherwise protected against degradation in the gut. For example, such pharmaceutical compositions may contain a membrane permeability enhancing agent, a protease enzyme inhibitor, and be enveloped by an enteric coating.

The pharmaceutical compositions comprise from approximately 1% to approximately 95% active ingredient. Unit dose forms are, for example, ampoules, vials, inhalers, eye drops and the like.

The pharmaceutical compositions of the present invention are prepared in a manner known per se, for example by means of conventional mixing, dissolving or lyophilizing processes.

Preference is given to the use of solutions of the active ingredient, and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80™ (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms. The alcohol component of these fatty acid esters has a maximum of 6 carbon atoms and is a monovalent or polyvalent, for example a mono-, di- or trivalent, alcohol, especially glycol and glycerol. As mixtures of fatty acid esters, vegetable oils such as cottonseed oil, almond oil, olive oil, castor oil, sesame oil, soybean oil and groundnut oil are especially useful.

The manufacture of injectable preparations is usually carried out under sterile conditions, as is the filling, for example, into ampoules or vials, and the sealing of the containers.

For parenteral administration, aqueous solutions of the active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Compositions for inhalation can be administered in aerosol form, as sprays, mist or in form of drops. Aerosols are prepared from solutions or suspensions that can be delivered with a metered-dose inhaler or nebulizer, i.e. a device that delivers a specific amount of medication to the airways or lungs using a suitable propellant, e.g. dichlorodifluoro-methane, trichlorofluoromethane, dichlorotetrafluoroethane, carbon dioxide or other suitable gas, in the form of a short burst of aerosolized medicine that is inhaled by the patient. It is also possible to provide powder sprays for inhalation with a suitable powder base such as lactose or starch.

Eye drops are preferably isotonic aqueous solutions of the active ingredient comprising suitable agents to render the composition isotonic with lacrimal fluid (295-305 mOsm/l). Agents considered are sodium chloride, citric acid, glycerol, sorbitol, mannitol, ethylene glycol, propylene glycol, dextrose, and the like. Furthermore the composition comprise buffering agents, for example phosphate buffer, phosphate-citrate buffer, or Tris buffer (tris(hydroxymethyl)-aminomethane) in order to maintain the pH between 5 and 8, preferably 7.0 to 7.4. The compositions may further contain antimicrobial preservatives, for example parabens, quaternary ammonium salts, such as benzalkonium chloride, polyhexamethylene biguanidine (PHMB) and the like. The eye drops may further contain xanthan gum to produce gel-like eye drops, and/or other viscosity enhancing agents, such as hyaluronic acid, methylcellulose, polyvinylalcohol, or polyvinylpyrrolidone.

### Use of Artificial Transcription Factors in a Method of Treatment

Furthermore the invention relates artificial transcription factors directed against promoters of genes involved in maladapted wound healing in the eye for use in influencing the cellular response following local injury in the eye. Likewise the invention relates to a method of treating fibrocontractive retinal disorders such as epiretinal gliosis, proliferative vitreoretinopathy, proliferative diabetic retinopathy and epiretinal membrane and glaucoma surgery connected fibroplasia comprising administering a therapeutically effective amount of an artificial transcription factor directed to the promoter of a gene involved in maladapted wound healing in the eye to a patient in need thereof. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

### Examples

### Cloning of DNA Plasmids

For all cloning steps, restriction endonucleases and T4 DNA ligase are purchased from New England Biolabs. Shrimp Alkaline Phosphatase (SAP) is from Promega. The high-fidelity Platinum Pfx DNA polymerase (Invitrogen) is applied in all standard PCR reactions. DNA fragments and plasmids are isolated according to the manufacturer's instructions using NucleoSpin Gel and PCR Clean-up kit, NucleoSpin Plasmid kit, or NucleoBond Xtra Midi Plus kit (Macherey-Nagel). Oligonucleotides are purchased from Sigma-Aldrich. All relevant DNA sequences of newly generated plasmids were verified by sequencing (Microsynth).

### Cloning of Hexameric Zinc Finger Protein Libraries for Yeast One Hybrid

Hexameric zinc finger protein libraries containing GNN and/or CNN and/or ANN binding zinc finger (ZF) modules are cloned according to Gonzalez B. et al., 2010, Nat Protoc 5, 791-810 with the following improvements. DNA sequences coding for GNN, CNN and ANN ZF modules were synthesized and inserted into pUC57 (GenScript) resulting in pAN1049 (SEQ ID NO: 170), pAN1073 (SEQ ID NO: 171) and pAN1670 (SEQ ID NO: 172), respectively. Stepwise assembly of zinc finger protein (ZFP) libraries is done in pBluescript SK (+) vector. To avoid insertion of multiple ZF modules during each individual cloning step leading to non-functional proteins, pBluescript (and its derived products containing 1ZFP, 2ZFPs, or 3ZFPs) and pAN1049, pAN1073 or pAN1670 are first incubated with one restriction enzyme and afterwards treated with SAP. Enzymes are removed using NucleoSpin Gel and PCR Clean-up kit before the second restriction endonuclease is added.

Cloning of pBluescript-1ZFPL is done by treating 5 μg pBluescript with Xhol, SAP and subsequently Spel. Inserts are generated by incubating 10 μg pAN1049 (release of 16 different GNN ZF modules) or pAN1073 (release of 15 different CNN ZF modules) or pAN1670 (release of 15 different ANN ZF modules) with Spel, SAP and subsequently Xhol. For generation of pBluescript-2ZFPL and pBluescript-3ZFPL, 7 μg pBluescript-1ZFPL or pBluescript-2ZFPL are cut with Agel, dephosphorylated, and cut with Spel. Inserts are obtained by applying Spel, SAP, and subsequently Xmal to 10 μg pAN1049 or pAN1073 or pAN1670, respectively. Cloning of pBluescript-6ZFPL was done by treating 14 μg of pBluescript-3ZFPL with Agel, SAP, and thereafter Spel to obtain cut vectors. 3ZFPL inserts were released from 20 μg of pBluescript-3ZFPL by incubating with Spel, SAP, and subsequently Xmal.

Ligation reactions for libraries containing one, two, and three ZFPs were set up in a 3:1 molar ratio of insert:vector using 200 ng cut vector, 400 U T4 DNA ligase in 20 μl total volume at RT (room temperature) overnight. Ligation reactions of hexameric zinc finger protein libraries included 2000 ng pBluescript-3ZFPL, 500 ng 3ZFPL insert, 4000 U T4 DNA ligase in 200 μl total volume, which were divided into ten times 20 μl and incubated separately at RT over night. Portions of ligation reactions were transformed into Escherichia coli by several methods depending on the number of clones required for each library. For generation of pBluescript-1ZFPL and pBluescript-2ZFPL, 3 μl of ligation reaction were directly used for heat shock transformation of E. coli NEB 5-alpha. Plasmid DNA of ligation reactions of pBluescript-3ZFPL was purified using NucleoSpin Gel and PCR Clean-up kit and transformed into electrocompetent E. coli NEB 5-alpha (EasyjecT Plus electroporator from EquiBio or Multiporator from Eppendorf, 2.5 kV and 25 μF, 2 mm electroporation cuvettes from Bio-Rad). Ligation reactions of pBluescript-6ZFP libraries were applied to NucleoSpin Gel and PCR Clean-up kit and DNA was eluted in 15 μl of deionized water. About 60 ng of desalted DNA were mixed with 50 μl NEB 10-beta electrocompetent E. coli (New England Biolabs) and electroporation was performed as recommended by the manufacturer using EasyjecT Plus or Multiporator, 2.5 kV, 25 μF and 2 mm electroporation cuvettes. Multiple electroporations were performed for each library and cells were directly pooled afterwards to increase library size. After heat shock transformation or electroporation, SOC medium was applied to the bacteria and after 1 h of incubation at 37° C. and 250 rpm, 30 μl of SOC culture were used for serial dilutions and plating on LB plates containing ampicillin. The next day, total number of obtained library clones was determined. In addition, ten clones of each library were chosen to isolate plasmid DNA and to check incorporation of inserts by restriction enzyme digestion. At least three of these plasmids were sequenced to verify diversity of the library. The remaining SOC culture was transferred to 100 ml LB medium containing ampicillin and cultured over night at 37° C. and 250 rpm. Those cells were used to prepare plasmid Midi DNA for each library.

For yeast one hybrid screens, hexameric zinc finger protein libraries are transferred to a compatible prey vector. For that purpose, the multiple cloning site of pGAD10 (Clontech) was modified by cutting the vector with Xhol/EcoRI and inserting annealed oligonucleotides OAN971 (TCGACAGGCCCAGGCGGCCCTCGAGGATATCATGATG ACTAGTGGCCAGGCCGGCCC, SEQ ID NO: 173) and OAN972 (AATTGGGCCGGC CTGGCCACTAGTCATCATGATATCCTCGAGGGCCGCCTGGGCCTG, SEQ ID NO: 174). The resulting vector pAN1025 (SEQ ID NO: 175) was cut and dephosphorylated, 6ZFP library inserts were released from pBluescript-6ZFPL by Xhol/Spel. Ligation reactions and electroporations into NEB 10-beta electrocompetent E. coli were done as described above for pBluescript-6ZFP libraries.

For improved yeast one hybrid screening, hexameric zinc finger libraries are also transferred into an improved prey vector pAN1375 (SEQ ID NO: 176). This prey vector was constructed as follows: pRS315 (SEQ ID NO: 177) was cut Apal/Narl and annealed OAN1143 (CGCCGCATGCATTCATGCAGGCC, SEQ ID NO: 178) and OAN1144 (TGCATGAATGCATGCGG, SEQ ID NO: 179) were inserted yielding pAN1373 (SEQ ID NO: 180). A Sphl insert from pAN1025 was ligated into pAN1373 cut with Sphl to obtain pAN1375.

For further improved yeast one hybrid screening, hexameric zinc finger libraries are also transferred into an improved prey vector pAN1920 (SEQ ID NO: 181).

For even further improved yeast one hybrid screening, hexameric zinc finger libraries are inserted into prey vector pAN1992 (SEQ ID NO: 182).

### Cloning of Bait Plasmids for Yeast One Hybrid Screening

For each bait plasmid, a 60 bp sequence containing a potential artificial transcription factor target site of 18 bp in the center is selected and a Ncol site is included for restriction analysis. Oligonucleotides are designed and annealed in such a way to produce 5′ HindIII and 3′ Xhol sites which allowed direct ligation into pAbAi (Clontech) cut with HindIII/Xhol. Digestion of the product with Ncol and sequencing are used to confirm assembly of the bait plasmid.

### Yeast Strain and Media

Saccharomyces cerevisiae Y1H Gold was purchased from Clontech, YPD medium and YPD agar from Carl Roth. Synthetic drop-out (SD) medium contained 20 g/l glucose, 6.8 g/l Na₂HPO₄.2H₂O, 9.7 g/l NaH₂PO₄.2H₂O (all from Carl Roth), 1.4 g/l yeast synthetic drop-out medium supplements, 6.7 g/l yeast nitrogen base, 0.1 g/l L-tryptophan, 0.1 g/l L-leucine, 0.05 g/l L-adenine, 0.05 g/l L-histidine, 0.05 g/l uracil (all from Sigma-Aldrich). SD-U medium contained all components except uracil, SD-L was prepared without L-leucine. SD agar plates did not contain sodium phosphate, but 16 g/l Bacto Agar (BD). Aureobasidin A (AbA) was purchased from Clontech.

### Preparation of Bait Yeast Strains

About 5 μg of each bait plasmid are linearized with BstBl in a total volume of 20 μl and half of the reaction mix is directly used for heat shock transformation of S. cerevisiae Y1H Gold. Yeast cells are used to inoculate 5 ml YPD medium the day before transformation and grown over night on a roller at RT. One milliliter of this pre-culture is diluted 1:20 with fresh YPD medium and incubated at 30° C., 225 rpm for 2-3 h. For each transformation reaction 1 OD₆₀₀ cells are harvested by centrifugation, yeast cells are washed once with 1 ml sterile water and once with 1 ml TE/LiAc (10 mM Tris/HCl, pH 7.5, 1 mM EDTA, 100 mM lithium acetate). Finally, yeast cells are resuspended in 50 μl TE/LiAc and mixed with 50 μg single stranded DNA from salmon testes (Sigma-Aldrich), 10 μl of BstBl-linearized bait plasmid (see above), and 300 μl PEG/TE/LiAc (10 mM Tris/HCl, pH 7.5, 1 mM EDTA, 100 mM lithium acetate, 50% (w/v) PEG 3350). Cells and DNA are incubated on a roller for 20 min at RT, afterwards placed into a 42° C. water bath for 15 min. Finally, yeast cells are collected by centrifugation, resuspended in 100 μl sterile water and spread onto SD-U agar plates. After 3 days of incubation at 30° C. eight clones growing on SD-U from each transformation reaction are chosen to analyze their sensitivity towards aureobasidin A (AbA). Pre-cultures were grown over night on a roller at RT. For each culture, OD₆₀₀ was measured and OD₆₀₀=0.3 was adjusted with sterile water. From this first dilution five additional 1:10 dilution steps were prepared with sterile water. For each clone 5 μl from each dilution step were spotted onto agar plates containing SD-U, SD-U 100 ng/ml AbA, SD-U 150 ng/ml AbA, and SD-U 200 ng/ml AbA. After incubation for 3 days at 30° C., three clones growing well on SD-U and being most sensitive to AbA are chosen for further analysis. Stable integration of bait plasmid into yeast genome is verified by Matchmaker Insert Check PCR Mix 1 (Clontech) according to the manufacturer's instructions. One of three clones is used for subsequent Y1H screen.

Transformation of Bait Yeast Strain with Hexameric Zinc Finger Protein Library

About 500 μl of yeast bait strain pre-culture are diluted into 1 l YPD medium and incubated at 30° C. and 225 rpm until OD₆₀₀=1.6-2.0 (circa 20 h). Cells are collected by centrifugation in a swing-out rotor (5 min, 1500×g, 4° C.). Preparation of electrocompetent cells is done according to Benatuil L. et al., 2010, Protein Eng Des Sel 23, 155-159. For each transformation reaction, 400 μl electrocompetent bait yeast cells are mixed with 1 μg prey plasmids encoding 6ZFP libraries and incubated on ice for 3 min. Cell-DNA suspension is transferred to a pre-chilled 2 mm electroporation cuvette. Multiple electroporation reactions (EasyjecT Plus electroporator or Multiporator, 2.5 kV and 25 μF) are performed until all yeast cell suspension has been transformed. After electroporation yeast cells are transferred to 100 ml of 1:1 mix of YPD:1 M Sorbitol and incubated at 30° C. and 225 rpm for 60 min. Cells are collected by centrifugation and resuspended in 1-2 ml of SD-L medium. Aliquots of 200 μl are spread on 15 cm SD-L agar plates containing 1000-4000 ng/ml AbA. In addition, 50 μl of cell suspension are used to make 1/100 and 1/1000 dilutions and 50 μl of undiluted and diluted cells are plated on SD-L. All plates are incubated at 30° C. for 3 days. The total number of obtained clones is calculated from plates with diluted transformants. While SD-L plates with undiluted cells indicate growth of all transformants, AbA-containing SD-L plates only resulted in colony formation if the prey 6ZFP bound to its bait target site successfully.

### Verification of Positive Interactions and Recovery of 6ZFP-Encoding Prey Plasmids

For initial analysis, forty good-sized colonies are picked from SD-L plates containing the highest AbA concentration and yeast cells were restreaked twice on SD-L with 1000-4000 ng/ml AbA to obtain single colonies. For each clone, one colony is used to inoculate 5 ml SD-L medium and cells are grown at RT overnight. The next day, OD₆₀₀=0.3 is adjusted with sterile water, five additional 1/10 dilutions are prepared and 5 μl of each dilution step are spotted onto SD-L, SD-L 500 ng/ml AbA, 1000 ng/ml AbA, SD-L 1500 ng/ml AbA, SD-L 2000 ng/ml AbA, SD-L 2500 ng/ml AbA, SD-L 3000 ng/ml AbA, and SD-L 4000 ng/ml AbA plates. Clones are ranked according to their ability to grow on high AbA concentration. From best growing clones 5 ml of initial SD-L pre-culture are used to spin down cells and to resuspend them in 100 μl water or residual medium. After addition of 50 U lyticase (Sigma-Aldrich, L2524) cells are incubated for several hours at 37° C. and 300 rpm on a horizontal shaker. Generated spheroblasts are lysed by adding 10 μl 20% (w/v) SDS solution, mixed vigorously by vortexing for 1 min and frozen at −20° C. for at least 1 h. Afterwards, 250 μl A1 buffer from NucleoSpin Plasmid kit and one spatula tip of glass beads (Sigma-Aldrich, G8772) are added and tubes are mixed vigorously by vortexing for 1 min. Plasmid isolation is further improved by adding 250 μl A2 buffer from NucleoSpin Plasmid kit and incubating for at least 15 min at RT before continuing with the standard NucleoSpin Plasmid kit protocol. After elution with 30 μl of elution buffer 5 μl of plasmid DNA are transformed into E. coli DH5 alpha by heat shock transformation. Two individual colonies are picked from ampicillin-containing LB plates, plasmids are isolated and library inserts are sequenced. Obtained results are analyzed for consensus sequences among the 6ZFPs for each target site.

### Cloning of a Reporter Plasmid for the Generation of Stable Luciferase/Secreted Alkaline Phosphatase Reporter Cell Lines for Testing Transducible Artificial Transcription Factor Activity

To generate a reporter construct containing Gaussia luciferase under the control of a hybrid CMV/artificial transcription factor target site promoter together with secreted alkaline phosphatase under control of the constitutive CMV promoter, 42 bp containing the artificial transcription factor binding site were cloned Af/III/Spel into pAN1660 (SEQ ID NO: 183). These reporter constructs contain a FlpIn site for stable integration into FlpIn site containing cells such as HEK 293 FlpIn TRex (Invitrogen) cells.

### Determination of Gene Expression Levels by Quantitative RT-PCR

Total RNA is isolated from cells using the RNeasy Plus Mini Kit (Qiagen, Hilden, Germany) according to the manufacturer's instructions. Frozen cell pellets are resuspended in RLT Plus Lysis buffer containing 10 μl/mL β-mercaptoethanol. After homogenization using QIAshredder spin columns, total lysate is transferred to gDNA Eliminator spin columns to eliminate genomic DNA. One volume of 70% ethanol is added and total lysate is transferred to RNeasy spin columns. After several washing steps, RNA is eluted in a final volume of 30 μl RNase free water. RNA is stored at −80° C. until further use. Synthesis of cDNA is performed using the High Capacity cDNA Reverse Transcription Kit (Applied Biosystems, Branchburg, N.J., USA) according to the manufacturer's instructions. cDNA synthesis is carried out in 20 μl of total reaction volume containing 2 μl 10× Buffer, 0.8 μl 25× dNTP Mix, 2 μl 10×RT Random Primers, 1 μl Multiscribe Reverse Transcriptase and 4.2 μl H₂O. A final volume of 10 μl RNA is added and the reaction is performed under the following conditions: 10 minutes at 25° C., followed by 2 hours at 37° C. and a final step of 5 minutes at 85° C. Quantitative PCR is carried out in 20 μl of total reaction volume containing 1 μL 20× TaqMan Gene Expression Master Mix, 10.0 μl TaqMan® Universal PCR Master Mix (both Applied Biosystems, Branchburg, N.J., USA) and 8 μl H₂O. For each reaction 1 μl of cDNA is added. qPCR is performed using the ABI PRISM 7000 Sequence Detection System (Applied Biosystems, Branchburg, N.J., USA) under the following conditions: an initiation step for 2 minutes at 50° C. is followed by a first denaturation for 10 minutes at 95° C. and a further step consisting of 40 cycles of 15 seconds at 95° C. and 1 minute at 60° C.

### Cloning of Artificial Transcription Factors for Mammalian Transfection

DNA fragments encoding polydactyl zinc finger proteins are cloned using standard procedures (Agel/Xhol) into mammalian expression vectors for expression in mammalian cells as fusion proteins between the zinc finger array of interest, a SV40 NLS, a 3× myc epitope tag and a N-terminal KRAB domain (pAN1255-SEQ ID NO: 184), a C-terminal KRAB domain (pAN1258-SEQ ID NO: 185), a SID domain (pAN1257-SEQ ID NO: 186) or a VP64 activating domain (pAN1510-SEQ ID NO: 187).

Plasmids for the generation of stably transfected, tetracycline-inducible cells were generated as follows: DNA fragments encoding artificial transcriptions factors comprising polydactyl zinc finger domain, a regulatory domain (N-terminal KRAB, C-terminal KRAB, SID or VP64), and a SV40 NLS are cloned into pAN2071 (SEQ ID NO: 188) using EcoRV/Agel. These artificial transcription factor expression plasmids can be integrated into the human genome into the AAVS1 locus by co-transfection with AAVS1 Left TALEN and AAVS1 Right TALEN (GeneCopoeia).

### Cell Culture and Transfections

HeLa cells are grown in Dulbecco's Modified Eagle's Medium (DMEM) supplemented with 4.5 g/l glucose, 10% heat-inactivated fetal bovine serum, 2 mM L-glutamine, and 1 mM sodium pyruvate (all from Sigma-Aldrich) in 5% CO₂ at 37° C. For luciferase reporter assay, 7000 HeLa cells/well are seeded into 96 well plates. Next day, co-transfections are performed using Effectene Transfection Reagent (Qiagen) according to the manufacturer's instructions. Plasmid midi preparations coding for artificial transcription factor and for luciferase are used in the ratio 3:1. Medium is replaced by 100 μl per well of fresh DMEM 6 h and 24 h after transfection.

### Generation and Maintenance of Flp-In™ T-ReX™ 293 Expression Cell Lines

Stable, tetracycline inducible Flp-In™ T-Rex™ 293 expression cell lines are generated by Flp Recombinase-mediated integration. Using Flp-In™ T-Rex™ Core Kit, the Flp-In™ T-Rex™ host cell line is generated by transfecting pFRT/lacZeo target site vector and pcDNA6/TR vector. For generation of inducible 293 expression cell lines, the pcDNA5/FRT/TO expression vector containing the gene of interest is integrated via Flp recombinase-mediated DNA recombination at the FRT site in the Flp-In™ T-Rex™ host cell line. Stable Flp-In™ T-Rex™ expression cell lines are maintained in selection medium containing (DMEM; 10% Tet-FBS; 2 mM glutamine; 15 μg/ml blasticidine and 100 μg/ml hygromycin). For induction of gene expression tetracycline is added to a final concentration of 1 μg/mL.

### Generation and Maintenance of Stably Artificial Transcription Factor Expressing Cell Lines Using TALENs

To generate cell lines stably expressing artificial transcription factors under the control of a tetracycline-inducible promoter, cells are co-transfected with a pAN2071-based expression construct containing the artificial transcription factor of interest and AAVS1 Left TALEN and AAVS1 Right TALEN (GeneCopoeia) plasmids using Effectene (Qiagen) transfection reagent) according to the manufacturer's recommendations. 8 hours post-transfection, growth medium was aspirated, cells were washed with PBS and fresh growth medium was added. 24 h post transfection cells were split at a ratio of 1:10 in growth medium containing Tet-approved FBS (tetracycline free FBS, Takara) without antibiotics. 48 h post-transfection, puromycin selection was started at cell-type specific concentration and cells were kept under selection pressure for 7-10 days. Colonies of stable cells were pooled and maintained in selection medium.

### Cloning of Artificial Transcription Factors for Bacterial Expression

DNA fragments encoding artificial transcription factors are cloned using standard procedures with EcoRV/Notl into bacterial expression vector pAN983 (SEQ ID NO: 189) based on pET41a+ (Novagen) for expression in E. coli as His₆-tagged fusion proteins between the artificial transcription factor and the TAT protein transduction domain.

### Production of Artificial Transcription Factor Protein

E. coli BL21(DE3) transformed with expression plasmid for a given artificial transcription factor were grown in 1 l LB media supplemented with 100 μM ZnCl₂ until OD₆₀₀ between 0.8 and 1 was reached, and induced with 1 mM IPTG for two hours. Bacteria were harvested by centrifugation, bacterial lysate was prepared by sonication, and inclusion bodies were purified. To this end, inclusion bodies were collected by centrifugation (5000 g, 4° C., 15 minutes) and washed three times in 20 ml of binding buffer (50 mM HEPES, 500 mM NaCl, 10 mM imidazole; pH 7.5). Purified inclusion bodies were solubilized on ice for one hour in 30 ml of binding buffer A (50 mM HEPES, 500 mM NaCl, 10 mM imidazole, 6 M GuHCl; pH 7.5). Solubilized inclusion bodies were centrifuged for 40 minutes at 4° C. and 13′000 g and filtered through 0.45 μm PVDF filter. His-tagged artificial transcription factors were purified using His-Trap columns on an Äktaprime FPLC (GEHealthcare) using binding buffer A and elution buffer B (50 mM HEPES, 500 mM NaCl, 500 mM imidazole, 6 M GuHCl; pH 7.5). Fractions containing purified artificial transcription factor were pooled and dialyzed at 4° C. overnight against buffer S (50 mM Tris-HCl, 500 mM NaCl, 200 mM arginine, 100 μM ZnCl₂, 5 mM GSH, 0.5 mM GSSG, 50% glycerol; pH 7.5) in case the artificial transcription factor contained a SID domain, or against buffer K (50 mM Tris-HCl, 300 mM NaCl, 500 mM arginine, 100 μM ZnCl₂, 5 mM GSH, 0.5 mM GSSG, 50% glycerol; pH 8.5) for KRAB domain containing artificial transcription factors. Following dialysis, protein samples were centrifuged at 14′000 rpm for 30 minutes at 4° C. and sterile filtered using 0.22 μm Millex-GV filter tips (Millipore). For artificial transcription factors containing VP64 activation domain, the protein was produced from the soluble fraction (binding buffer: 50 mM NaPO₄ pH 7.5, 500 mM NaCl, 10 mM imidazole; elution buffer 50 mM HEPES pH 7.5, 500 mM NaCl, 500 mM imidazole) using His-Bond Ni-NTA resin (Novagen) according to manufactures recommendation. Protein was dialyzed against VP64-buffer (550 mM NaCl pH 7.4, 400 mM arginine, 100 μM ZnCl₂).

### Determination of DNA Binding Activity of Artificial Transcription Factors Using ELDIA (Enzyme-Linked DNA Interaction Assay)

BSA pre-blocked nickel coated plates (Pierce) are washed 3 times with wash buffer (25 mM Tris/HCl pH 7.5, 150 mM NaCl, 0.1% BSA, 0.05% Tween-20). Plates are coated with purified artificial transcription factor under saturating conditions (50 pmol/well) in storage buffer and incubated 1 h at RT with slight shake. After 3 washing steps, 1×10⁻¹² to 5×10⁻⁷ M of annealed, biotinylated oligos containing 60 bp promoter sequence are incubated in binding buffer (10 mM Tris/HCl pH 7.5, 60 mM KCl, 1 mM DTT, 2% glycerol, 5 mM MgCl₂ and 100 μM ZnCl₂) in the presence of unspecific competitor (0.1 mg/ml ssDNA from salmon sperm, Sigma) with the bound artificial transcription factor for 1 h at RT. After washing (5 times), wells are blocked with 3% BSA for 30 minutes at RT. Anti-streptavidin-HRP is added in binding buffer for 1 h at RT. After 5 washing steps, TMB substrate (Sigma) is added and incubated for 2 to 30 minutes at RT. Reaction is stopped by addition of TMB stop solution (Sigma) and sample extinction is read at 450 nm. Data analysis of ligand binding kinetics is done using Sigma Plot V8.1 according to Hill.

### Protein Transduction

Cells grown to about 80% confluency are treated with 0.01 to 1 μM artificial transcription factor or mock treated for 2 h to 120 h with optional addition of artificial transcription factor every 24 h in OptiMEM or growth media at 37° C. Optionally, 10-500 μM ZnCl₂ are added to the growth media. For immunofluorescence, cells are washed once in PBS, trypsinized and seeded onto glass cover slips for further examination.

### Immunofluorescence

Cells are fixed with 4% paraformaldehyde in PBS, treated with 0.15% Triton X-100 for 15 minutes, blocked with 10% BSA/PBS and incubated overnight with mouse anti-HA antibody (1:500, H9658, Sigma) or mouse anti-myc (1:500, M5546, Sigma). Samples are washed three times with PBS/1% BSA, and incubated with goat anti-mouse antibodies coupled to Alexa Fluor 546 (1:1000, Invitrogen) and counterstained using DAPI (1:1000 of 1 mg/ml for 3 minutes, Sigma). Samples are analyzed using fluorescence microscopy.

### Combined Luciferase/SEAP Promoter Activity Assay

To test activity of artificial transcription factors, a reporter cell line was employed. This reporter cell line is based on HEK 293 FlpIn TRex cells containing Gaussia luciferase under control of a hybrid CMV/artificial transcription factor target site promoter and secreted alkaline phosphatase under control of a constitutive CMV promoter.

1×10⁵ reporter cells/well are seeded in 6-well plates 24 h before protein transduction. 24 h after seeding, medium is aspirated from the plate and cells are washed 1× with PBS. For protein treatment, the artificial transcription factor or control protein is diluted to a final concentration of 1 μM in OptiMEM, added to the cells and incubated for 2 h in an incubator (37° C.; 5% CO₂). Following protein transduction, cells were grown for 24 h in normal growth medium. Supernatant was transferred to 96 well plates, and centrifuged at 2000 rpm for 5 min. For measurement of Gaussia Luciferase the Pierce™ Gaussia Luciferase Glow Assay Kit (Thermo Scientific) was used according to manufacturer's instructions. The working solution was equilibrated to room temperature and coelenterazine was added at a dilution of 1:100. 20 μl of cell supernatant was transferred into an opaque 96-well plate and 50 μl of working solution was added. After 10 min of incubation luminescence was measured using MicroLumatPlus (Berthold Technologies) at an integration time of 1.0 s. For measurement of secreted alkaline phosphatase activity the chemiluminescent SEAP Reporter Gene Assay (Roche) was used according to manufacturer's instructions. Cell supernatant was diluted 1:4 with dilution buffer and heat inactivated at 65° C. for 5 min. 50 μL of heat inactivated sample was transferred to a an opaque 96-well plate and 50 μL of inactivation buffer was added. After incubation for 5 min at room temperature, 50 μL of substrate reagent, consisting of AP Substrate 1:20 in substrate buffer, was added and incubated for 10 min at room temperature under gentle agitation. Luminescence was measured using MicroLumatPlus (Berthold Technologies) at an integration time of 1.0 s.

### Measuring Cellular Impedance or ECIS

Cells treated with artificial transcription factor or control protein are grown in 8-well line array or 8W1E ECIS cultureware (Applied BioPhysics) for 24 hours or until impedance has reached a plateau. Wounding of the cell layer is induced using the wounding protocol of the ECIS ZTheta (Applied BioPhysics), and healing is followed in real time for 24 hours or until impedance of the control treated cells reach a plateau.

1. An artificial transcription factor comprising a polydactyl zinc finger protein targeting specifically a promoter of a gene involved in maladapted wound healing in the eye fused to an activatory or inhibitory protein domain, a nuclear localization sequence, a protein transduction domain and optionally an endosome-specific protease recognition site.

2. The artificial transcription factor according to claim 1 comprising an endosome-specific protease recognition site.

3. The artificial transcription factor according to claim 1 comprising a zinc finger protein of a protein sequence selected from the group consisting of SEQ ID NO: 88 to 128.

4. The artificial transcription factor according to claim 1 comprising a zinc finger protein of a protein sequence selected from the group consisting of SEQ ID NO: 88 to 128, wherein up to three individual zinc finger modules are exchanged against other zinc finger modules with alternative binding characteristic and/or wherein up to twelve individual amino acids are exchanged.

5. The artificial transcription factor according to claim 1 further comprising a polyethylene glycol residue.

6. A pharmaceutical composition comprising an artificial transcription factor according to claim 1.

7. The artificial transcription factor according to claim 1 for use in increasing or decreasing the expression from a gene promoter.

8. The artificial transcription factor according to claim 1 for the treatment of fibrocontractive retinal disorders.

9. The artificial transcription factor according to claim 1 for the treatment of epiretinal gliosis, proliferative vitreoretinopathy, proliferative diabetic retinopathy and/or epiretinal membrane.

10. The artificial transcription factor according to claim 1 for the treatment of fibroplasia following glaucoma surgery.

11. A method of treating fibroplasia and fibrocontractive retinal disorders comprising administering a therapeutically effective amount of an artificial transcription factor according to claim 1 to a patient in need thereof.
